(19) United States
(12) Patent Application Publication
(10) Pub. No.: US 2016/0281166 A1
(43) Pub. Date: Sep. 29, 2016

(54) METHODS AND SYSTEMS FOR SCREENING DISEASES IN SUBJECTS

(71) Applicant: PARABASE GENOMICS, INC., Boston, MA (US)

(72) Inventors: Arindam BHATTACHARJEE, Andover, MA (US); Tanya SOKOLSKY, Cambridge, MA (US); Edwin NAYLOR, Mt. Pleasant, SC (US); Richard B. PARAD, Newton, MA (US); Evan MAUCELI, Roslindale, MA (US)

(21) Appl. No.: 15/078,579

(22) Filed: Mar. 23, 2016

Related U.S. Application Data
(60) Provisional application No. 62/136,836, filed on Mar. 23, 2015.
(60) Provisional application No. 62/137,745, filed on Mar. 24, 2015.

(51) Int. Cl. C12Q 1/68; C40B 30/02; G06F 19/22

(52) U.S. Cl. CPC C12Q 1/6883; G06F 19/22; C40B 30/02; C12Q 2600/156; C12Q 2600/158

(57) ABSTRACT

The present disclosure provides systems, devices, and methods for a fast-turnaround, minimally invasive, and/or cost-effective assay for screening diseases, such as genetic disorders and/or pathogens, in subjects.

### CROSS-REFERENCE

This application claims priority to U.S. Provisional Patent Application 62/136,836, filed Mar. 23, 2015, and U.S. Provisional Patent Application 62/137,745, filed Mar. 24, 2015, which are entirely incorporated herein by reference.

### GOVERNMENT RIGHTS

The invention described herein was made with government support under phase I SBIR NIH grants from NIDCD (1R43DC013012-01) and NICHD (1R43HD076544-01) awarded by the National Institutes of Health. The United States Government has certain rights in the invention.

### BACKGROUND

For newborns with genetic disorders, a rapid diagnosis of diseases can make the difference between life and death and reduce length of stay in the neonatal intensive care unit (NICU). However, current single gene sequencing methods used for confirmatory diagnosis can be impractical in newborns. They can be costly, time consuming and require a large blood volume that cannot be easily or safely obtained from an infant.

Two compelling forces are expected to drive adoption of genetic testing in newborns. First is the need for rapid, minimally invasive diagnosis to treat and minimize adverse outcomes. Second is the financial incentive to shorten length of stay and reduce overall patient-management costs associated with delayed or inaccurate diagnosis. The methods and systems disclosed herein can provide a fast-turnaround, minimally invasive, and cost-effective assay for screening diseases, such as genetic disorders and/or pathogens, in newborns. It demonstrates that turnaround and sample requirements for newborn genetic cases can be achieved using Targeted Next-Generation Sequencing (TNGS), and that combining genetic etiology (via TNGS) with phenotype can allow a prompt and comprehensive clinical understanding.

### SUMMARY

The methods and systems disclosed herein can be used for detecting a genetic condition in a subject, comprising: (a) providing a sample previously obtained from the subject, wherein the sample comprises a dried blood spot (DBS) sample, a cord blood sample, single blood drop, saliva, oral swab, other bodily fluid or other tissue; (b) sequencing the sample to generate a sequencing product, wherein the sequencing product is determined by a sequencing method selected from a group consisting of next-generation sequencing (NGS), targeted next-generation sequencing (TNGS) and whole-exome sequencing (WES); and (c) analyzing the sequencing product to determine a presence of, absence of or predisposition to the genetic condition. In some cases, the methods and systems further comprise providing a sample previously obtained from a relative of the subject.

The methods and systems disclosed herein can also be used for detecting a pathogen in a subject, comprising: (a) providing a sample previously obtained from the subject, wherein the sample comprises a dried blood spot (DBS) sample, a cord blood sample, single blood drop, saliva, oral swab, other body fluid or other tissue; (b) sequencing the sample to generate a sequencing product, wherein the sequencing product is determined by a sequencing method selected from a group consisting of next-generation sequencing (NGS), targeted next-generation sequencing (TNGS) and whole-exome sequencing (WES); and (c) analyzing the sequencing product to determine a presence of, absence of or predisposition to the pathogen. In some cases, the methods and systems further comprise providing a sample previously obtained from a relative of the subject.

The methods and systems disclosed herein can also be used for detecting a hearing loss condition in a subject, comprising: (a) providing a sample previously obtained from the subject, wherein the sample comprises a dried blood spot (DBS) sample, a cord blood sample, single blood drop, saliva, oral swab, other body fluid or other tissue; (b) sequencing the sample to generate a sequencing product, wherein the sequencing product is determined by a sequencing method selected from a group consisting of next-generation sequencing (NGS), targeted next-generation sequencing (TNGS) and whole-exome sequencing (WES); and (c) analyzing the sequencing product to determine a presence of, absence of or predisposition to the hearing loss condition. In some cases, the methods and systems further comprise providing a sample previously obtained from a relative of the subject.

In an aspect, the subject disclosed herein is a fetus, a newborn, an infant, a child, an adolescent, a teenager or an adult. In some cases, the subject is a newborn. In some cases, the subject is within 28 days after birth. In some cases, the subject is a relative of a newborn.

In another aspect, the methods and systems disclosed herein use less than 1000 μL of the sample (e.g. DBS). For example, less than 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950 or 1000 μL of the sample (e.g. DBS) can be used.

In another aspect, the sample disclosed herein is a blood sample. In some cases, the blood sample is a dried blood spot (DBS) sample. In some cases, the sample contains less than 1000 μL of blood. For example, less than 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950 or 1000 μL of the sample (e.g. DBS) is contained within the sample. In some cases, the sample contains less than 50 μL of blood.

In another aspect, providing a sample further comprise purifying and/or isolating a DNA from the sample. In another aspect, providing a sample does not comprise purifying and/or isolating a DNA from the sample. In some cases, the sample is a whole blood sample. In some cases, the sample is a whole blood sample without purification. In some cases, the sample is a purified sample. In some cases, the sequencing the sample to generate a sequencing product is done with on a purified sample. In some cases, the sequencing the sample to generate a sequencing product is done with on a purified DNA sample. In some cases, the sequencing the sample to generate a sequencing product is done with on a whole blood sample. In some cases, the sequencing the sample to generate a sequencing product is done with on a whole blood sample without purification.

In another aspect, the disclosed methods and systems can be used to isolate more than 10 μg of DNA from a sample. For example, the disclosed methods and systems are used to isolate more than 1, 2, 3, 4, 5, 6, 7, 8, 9, 10, 20, 30, 40, 50, 60, 70, 80, 90, 100, 200, 300, 400, 500, 600, 700, 800, 900, or 1000 ng of DNA from a sample. In some cases, the disclosed methods and systems are used to isolate more than 1, 2, 3, 4, 5, 6, 7, 8, 9, or 10 μg of DNA from a sample. In some cases, the disclosed methods and systems are used to isolate more than 5, 6, 7, 8, 9, 10, 20, 30, 40, 50, 60, 70, 80, 90, 100, 200, 300, 400, 500, 600, 700, 800, or 900 pg of DNA from a sample. In typical cases, the disclosed methods and systems are used to isolate more than 100 ng of DNA from a sample.

In another aspect, the disclosed methods and systems can be used to isolate less than 10 μg of DNA from a sample. For example, the disclosed methods and systems are used to isolate less than 1, 3, 4, 5, 6, 7, 8, 9, 10, 20, 30, 40, 50, 60, 70, 80, 90, 100, 200, 300, 400, 500, 600, 700, 800, 900, or 1000 ng of DNA from a sample. In some cases, the disclosed methods and systems are used to isolate less than 1, 2, 3, 4, 5, 6, 7, 8, 9, or 10 μg of DNA from a sample. In typical cases, the disclosed methods and systems are used to isolate less than 1 μg of DNA from a sample.

In another aspect, the disclosed methods and systems can be used to isolate about 1 ng-10 μg of DNA from a sample. For example, the disclosed methods and systems are used to isolate about 1-700, 1-500, 1-300, 1-100, 1-80, 1-60, 1-40, 1-20, 1-10, 1-5, 10-700, 10-500, 10-300, 10-100, 10-80, 10-60, 10-40, 10-20, 20-700, 20-500, 20-300, 20-100, 20-80, 20-60, 20-40, 40-700, 40-500, 40-300, 40-100, 40-80, 40-60, 60-700, 60-500, 60-300, 60-100, 60-80, 80-700, 80-500, 80-300, 80-100, 100-700, 100-500, 100-300, 300-700, 300-500, or 500-700 ng of DNA from a sample. In typical cases, the disclosed methods and systems are used to isolate about 100-700 ng of DNA from a sample.

In another aspect, the disclosed methods and systems can be used to isolate more than 10 μg of DNA from a dried blood spot. For example, the disclosed methods and systems are used to isolate more than 1, 2, 3, 4, 5, 6, 7, 8, 9, 10, 20, 30, 40, 50, 60, 70, 80, 90, 100, 200, 300, 400, 500, 600, 700, 800, 900, or 1000 ng of DNA from a dried blood spot. In some cases, the disclosed methods and systems are used to isolate more than 1, 2, 3, 4, 5, 6, 7, 8, 9, or 10 μg of DNA from a dried blood spot. In some cases, the disclosed methods and systems are used to isolate more than 5, 6, 7, 8, 9, 10, 20, 30, 40, 50, 60, 70, 80, 90, 100, 200, 300, 400, 500, 600, 700, 800, or 900 pg of DNA from a dried blood spot. In typical cases, the disclosed methods and systems are used to isolate more than 100 ng of DNA from a dried blood spot.

In another aspect, the disclosed methods and systems can be used to isolate less than 10 μg of DNA from a dried blood spot. For example, the disclosed methods and systems are used to isolate less than 1, 2, 3, 4, 5, 6, 7, 8, 9, 10, 20, 30, 40, 50, 60, 70, 80, 90, 100, 200, 300, 400, 500, 600, 700, 800, 900, or 1000 ng of DNA from a dried blood spot. In some cases, the disclosed methods and systems are used to isolate less than 1, 2, 3, 4, 5, 6, 7, 8, 9, or 10 μg of DNA from a dried blood spot. In typical cases, the disclosed methods and systems are used to isolate less than 1 μg of DNA from a dried blood spot.

In another aspect, the disclosed methods and systems can be used to isolate about 1 ng-10 μg of DNA from a dried blood spot. For example, the disclosed methods and systems are used to isolate about 1-700, 1-500, 1-300, 1-100, 1-80, 1-60, 1-40, 1-20, 1-10, 1-5, 10-700, 10-500, 10-300, 10-100, 10-80, 10-60, 10-40, 10-20, 20-700, 20-500, 20-300, 20-100, 20-80, 20-60, 20-40, 40-700, 40-500, 40-300, 40-100, 40-80, 40-60, 60-700, 60-500, 60-300, 60-100, 60-80, 80-700, 80-500, 80-300, 80-100, 100-700, 100-500, 100-300, 300-700, 300-500, or 500-700 ng of DNA from a dried blood spot. In typical cases, the disclosed methods and systems are used to isolate about 100-700 ng of DNA from a dried blood spot.

In another aspect, the method disclosed herein sequences DNA. In some cases, the method disclosed herein uses double stranded DNA. In some cases, more than 10% of the sequenced DNA is double stranded DNA. For example, more than 10%, 15%, 20%, 25%, 30%, 35%, 40%, 45%, 50%, 55%, 60%, 65%, 70%, 75%, 80%, 85%, 90%, 95% or 99% of the sequenced DNA is double stranded DNA.

In another aspect, the method disclosed herein isolates DNA. In some cases, the method disclosed herein isolates double stranded DNA. In some cases, more than 10% of the isolated DNA is double stranded DNA. For example, more than 10%, 15%, 20%, 25%, 30%, 35%, 40%, 45%, 50%, 55%, 60%, 65% 70%, 75%, 80%, 85%, 90%, 95% or 99% of the isolated DNA is double stranded DNA.

In another aspect, the double stranded DNA is maintained and processed by an enzyme. In some cases, the double stranded DNA is fragmented by an enzyme. In some cases, the enzyme recognizes a methylation site. In some cases, the enzyme recognizes a ^{m}CNNR site. In some cases, the enzyme is MspJI. In some cases, more than 10% of the fragmented DNA is double stranded DNA. For example, more than 10%, 15%, 20%, 25%, 30%, 35%, 40%, 45%, 50%, 55%, 60%, 65%, 70%, 75%, 80%, 85%, 90%, 95% or 99% of the fragmented DNA is double stranded DNA.

In another aspect, the methods and systems disclosed herein can be used for detecting a genetic condition. In some cases, the genetic condition is caused by a genetic alteration. The genetic alteration can be in a nuclear gene(s). The genetic alteration can be in a mitochondrial gene(s). The genetic alteration can be in nuclear and mitochondrial genes. In some cases, the genetic condition is a hearing loss condition. In some cases, the hearing loss condition is caused by a genetic alteration. In some cases, the genetic alteration is selected from a group consisting of the following: nucleotide substitution, insertion, deletion, frameshift, nonframeshift, intronic, promoter, known pathogenic, likely pathogenic, splice site, gene conversion, modifier, regulatory, enhancer, silencer, synergistic, short tandem repeat, genomic copy number variation, causal variant, genetic mutation, and epigenetic mutation.

In another aspect, analyzing the sequencing product comprises determining a presence, absence or predisposition of the genomic copy number variation or the genetic mutation. In some cases, analyzing the sequencing product comprises determining a presence, absence, predisposition, and/or change in copy number of the genomic region or the genetic mutation. In some cases, the genetic mutation is a uniparental disomy, heterozygous, hemizygous or homozygous mutation.

In another aspect, the methods and systems disclosed herein further comprise verifying the genetic alteration with a clinical phenotype and/or with a Newborn Screening (NBS). In some cases, the methods and systems disclosed herein can further comprise verifying the genetic alteration following a presumptive positive identified by a Newborn Screening (NBS).

In another aspect, the methods and systems disclosed herein further comprise verifying cis- or trans-configuration of the heterozygous mutations using a next-generation sequencing (NGS) or an orthogonal method. In some cases, the orthogonal method is Sanger sequencing or a pooling strategy.

In another aspect, the methods and systems disclosed herein further comprise depleting human genome (e.g., endogenous) from the sequencing product. In some cases, the depleting human genome from the sequencing product is performed by a subtractive method. In some cases, the depleting human genome and its corresponding signal comprises in silico subtraction of the human genome. In some cases, the method of depleting human genome comprises contacting the DNA sample with an enzyme. In some cases, the enzyme is a duplex-specific nuclease (DSN). In some cases, the enzyme is MspJI. In some cases, the depleting human genome results in at least about 5-fold increase in number of reads the pathogen genome as compared to an uncontacted control. For example, the depleting human genome results in at least about 2-fold, 3-fold, 4-fold, 5-fold, 6-fold, 7-fold, 8-fold, 9-fold, or 10-fold increase in number of reads the pathogen genome as compared to an uncontacted control. In some cases, the method of depleting human genome comprises removal of specific cell types from blood or other body fluids. For example, white blood cells that harbor the human genome can be removed to enrich the non-endogenous and/or non-human (e.g., pathogen) fraction or cell-free fraction.

In another aspect, the methods and systems disclosed herein can be used for detecting a pathogen that causes a genetic condition (e.g., hearing loss) in the subject. In some cases, the pathogen is cytomegalovirus (CMV). In some cases, the hearing loss condition is caused by a cytomegalovirus (CMV) infection. In some cases, the pathogen causes sepsis in the subject.

In another aspect, the methods and systems disclosed herein can be used for a subject in a neonatal intensive care unit (NICU), pediatric center, pediatric clinic, referral center or referral clinic. In some cases, the neonatal intensive care unit (NICU), pediatric center, pediatric clinic, referral center or referral clinic is specialized in Cystic Fibrosis, metabolic, or hearing deficiency. In some cases, a Newborn Screening (NBS) has been performed on the subject. In some cases, a Newborn Screening (NBS) was performed on the subject, for example, within 1 hour, 3 hours, 6 hours, 12 hours, 1 day, 2 days, 3 days, 4 days, 5 days, 6 days, 7 days, 8 days, 9 days, 10 days, 12 days, 14 days, 16 days, 18 days, 20 days, 22 days, 24 days, 26 days, or 28 days after birth. In some cases, a Newborn Screening (NBS) has not been performed on the subject. In some cases, the subject has a phenotype. In some cases, the subject has a phenotype of a disease. In some cases, the subject has no phenotype. In some cases, the subject has no phenotype of a disease.

In another aspect, sequencing the DNA comprises sequencing at least one gene selected from the genes in Tables 3, 4, 13, 14, 15, 16, 17, 18, and 19. In some cases, sequencing the DNA comprises sequencing at least, for example, 1, 2, 3, 4, 5, 6, 7, 8, 9, 10, 20, 30, 40, 50, 60, 70, 80, 90, 100, 125, 150, 175, 200, 225, 250, 275, 300, 350, 400, 450, or 500 genes selected from the genes in Tables 3, 4, 13, 14, 15, 16, 17, 18, and 19. In typical cases, sequencing the DNA comprises sequencing at least five genes selected from the genes in Tables 3, 4, 13, 14, 15, 16, 17, 18, and 19. In some cases, sequencing the sample comprises conducting whole genome amplification on the sample. In some cases, sequencing the sample does not comprise conducting whole genome amplification on the sample. In some cases, the sample comprises less than 10, 20, 30, 40, 50, 60, 70, 80, 90, 100, 200, 300, 400, 500, 600, 700, 800, 900 or 1000 ng of DNA.

In another aspect, determining the presence, absence or predisposition of a genetic condition comprises determining the predisposition or susceptibility to the genetic condition. In another aspect, determining the presence, absence or predisposition of a genetic condition comprises determining the possibility of developing the genetic condition.

In another aspect, the genetic condition disclosed herein comprises a disease, a phenotype, a disorder, or a pathogen. In some cases, the disorder is a genetic disorder.

In another aspect, analyzing the sequencing product further comprises comparing the sequencing product with a database of neonatal specific variant annotation.

In another aspect, the methods and systems disclosed herein comprise a kit, comprising at least one capture probe targeting to at least one gene selected from the genes in Tables 3, 4, 13, 14, 15, 16, 17, 18, and 19. In some cases, the kit comprises at least, for example, 1, 10, 25, 50, 75, 100, 125, 150, 175, 200, 225, 250, 275, 300, 350, 400, 450, 500, 550, 600, 650, 700, 750, 800, 850, 900, 950, 1,000, 1100, 1200, 1300, 1400, 1500, 1600, 1700, 1800, 1900, 2,000, 3,000, 4,000, 5,000, 6,000, 7,000, 8,000, 9,000, 10,000, 15,000, 20,000, 30,000, 40,000, 50,000, 60,000, 70,000, 80,000, 90,000, or 100,000 capture probes. In some cases, the kit comprises at least one capture probe targeting to at least, for example, 1, 2, 3, 4, 5, 6, 7, 9, 10, 20, 30, 40, 50, 60, 70, 80, 90, 100, 125, 150, 175, 200, 225, 250, 275, 300, 350, 400, 450, or 500 genes selected from the genes in Tables 3, 4, 13, 14, 15, 16, 17, 18, and 19. In a typical case, the kit comprises at least one capture probe targeting to at least five genes selected from the genes in Tables 3, 4, 13, 14, 15, 16, 17, 18, and 19. In some cases, the at least one capture probe is used for solution hybridization or DNA amplification.

In another aspect, the kit comprises at least one support bearing the at least one capture probe. In some case, the at least one support is a microarray. In some case, the at least one support is a bead.

Disclosed herein can be also be a system comprising: a) a digital processing device comprising an operating system configured to perform executable instructions and a memory device; b) a computer program including instructions executable by the digital processing device to classify a sample from a subject or a relative of the subject comprising: i) a software module configured to receive a sequencing product from the sample from the subject or a relative of the subject; ii) a software module configured to analyze the sequencing product from the sample from the subject or a relative of the subject; and iii) a software module configured to determine a presence, absence or predisposition of a genetic condition. In some cases, the subject is a newborn. In some cases, the genetic condition comprises a genetic disorder, a pathogen or a hearing loss condition. In some cases, the software module is configured to automatically detect the presence, absence or predisposition of a genetic condition. In some cases, the system further comprises a software module configured to annotate the genetic condition and/or provide a treatment suggestion.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG.” and “FIGS.” herein), of which:

FIGS. 1A and 1B show an algorithm and workflow for next-generation sequencing (NGS)-based newborn confirmatory and diagnostic testing.

FIG. 2 shows a computer system that is programmed or otherwise configured to implement methods of the disclosure.

FIG. 3 shows a technique for calling variants in regions of high homology.

FIGS. 4A and 4B show the quality and performance of DNA isolated from bio-specimens. FIG. 4A: Agarose Gel QC of genomic DNA purified from DBS. DNA is high molecular weight and yield increases with spot area sampled. Sufficient yield is obtained from a single spot for NGS library preparation. FIG. 4B: TNGS performance metrics. DNA isolated from DBS, Whole Blood and Saliva of the same individual performs similarly in TNGS. Graphs show WES results for % Reads On-Target (Reads On-Target/Reads Mapped) and Coverage at least 1, 10, 20, 50 and 100 reads on target. NBDx panel capture results were also similar across bio-specimen types.

FIGS. 5A, 5B, and 5C show the performance of a newborn-specific targeted gene panel (NBDx) capture and sequencing. FIG. 5A shows the sensitivity plots for GCDH across chromosomal positions generated for WES and NBDx. FIG. 5B shows the sensitivity plots for PAH across chromosomal positions generated for WES and NBDx. FIG. 5C shows the coverage of approximately 6,215 ClinVar sites common to both WES and NBDx tiled regions.

FIGS. 6A and 6B show the uniformity of coverage and reproducibility of NBDx. Histogram of coverage counts for all bases in the tiled regions as generated by GATK's Base Coverage Distribution program. FIG. 6A shows NBDx and WES distribution for the respective target regions. FIG. 6B shows the representative pairwise-comparison of variant read depth. Read depth of variants in exons of the 126 NBS genes plotted for coverage depth from independent capture and sequencing runs of a single patient sample. Variants with ≧10 reads were included. The GATK pipeline coverage threshold was 200 reads. The same sample is compared pairwise for WES and NBDx capture (˜140 variants/sample).

FIG. 7 shows the variant management for filtering blinded samples (Table 1). Variant files (VCF) were loaded into Opal for annotation and filters applied in Variant Miner. A) Protein Impact were categorized as Stop Gained/Lost, Indel/Frameshift, Splice Site and Non-synonymous. B) Variant scoring used prediction algorithms including SIFT, PolyPhen, MutationTaster, PhyloP and Omicia Score (a random-forest classifier that creates an integrative score between 0-1). Databases include ClinVar, OMIM, PharmGKB, GWAS, Locus Specific Databases (from PhenCode), 1000 genomes, dbSNP, HGMD, LOVD, and an in-house database. Literature searches were also included to more fully understand the classification of filtered variants. Intronic mutations were annotated in Opal and identified through variant scoring following identification of a deleterious mutation with heterozygosity for a disorder indicated by the clinical summary.

FIG. 8 shows the enrichment of CMV from DBS isolated DNA.

FIGS. 9A and 9B show the hybrid capture performance of NBDx v1.0. FIG. 9A shows that the NBDx v1.0 panel has percent reads on target similar or greater than the Exome. FIG. 9B shows that the smaller target of the NBDx v1.0 allows for greater coverage depth with more samples run in unison as compared to the Exome.

FIG. 10 shows the concordance of Broad/Agilent and Parabase/Roche Exome Missense.

FIGS. 11A & 11B show fractions of ClinVar at various coverage depths and sequencing matrics for WES and NBDx. ClinVar sites were determined by intersecting NBDx tiled regions with the ClinVar trac kin UCSC browser. Duplicate entry removal gives a total of 6215 unique ClinVar sites. Coverage at each site was determined using Samtools Pileup and the number with coverage ≧X counted for X=10, 20, 50, and 100. The averages for 8 matched samples from WES and NBDx are shown. Sequencing mapped reads, reads on-target, average reads and specificity (mapped reads/reads on-target) were calculated from 8 WES and 32 NBDx runs.

FIG. 12 shows the Multi-Exon deletion detected in clinical case in Maple Syrup Urine Disease. Clinical phenotype was presented. WES had high overall target coverage (87% of target covered >20×), yet no causal variants detected by standard analysis of MSUD genes. Further analysis by normalization of this sample with an internal control confirmed capture performance and revealed a multi-exon deletion in BCKDHB.

FIG. 13 shows the library complexity. The plot estimates the return on investment for sequencing at higher coverage than the observed using Mark Duplicates in Picard (picard.sourceforge.net). Five samples run with both NBDx and WES are shown. Dashed lines indicate 95% saturation. Since WES has more target region the NBDx it takes longer to saturate, requiring more cost to reach the coverage achieved by NBDx.

FIG. 14 shows the overlay coverage plot of 5 samples across contiguous regions. Tiled regions with >95% sensitivity for heterozygous calls across the 126 NBS genes on Chromosome 3. Sample 10642 shows an intronic deletion in PCCB. Inset: Coverage depth across PCCB visualized in GenomeBrowse (www.goldenhelix.com). Top: Sample 10642; Middle: Sample 4963 (from the same multiplexed pool). Bottom: RefSeq exon map of PCCB. The red box highlights the PCCB deletion

FIG. 15 shows the performance comparison of archival DBS and degraded DNA from 10 mL whole blood. Comparisons (from left to right: Specificity; % Target at 1×; % Target at 20×; and % Target at 100×) are shown for NBDx captures. Specificity is On-Target Reads/Mapped Reads. WES, n=17; NBDx, n=39; WGA, n=2; Archival, n=4; Archival+WGA, n=24; Degraded, n=7. Archival DBS were stored up to 10 years at room temperature. Those passing QC (as described above) are categorized as “Archival”. Archival DBS with signs of degradation made use of an additional WGA step (“Archival+WGA”).

FIGS. 16A and 16B show homozygous variant calls from pooled samples. FIG. 16A shows Six individuals were analyzed independently for autosomal homozygous variants. The individuals were combined in three pools as shown and the homozygous variants were followed. Three unique homozygous mutations in GCDH, GALT and BTD from three different samples were followed as shown in B. AP samples are non-CSC samples. FIG. 16B shows The mixing experiment of samples showing the response of the three mutations allowed us to follow the expected vs observed proportions in each mutation i.e., dose response. The expected proportions of 0%, 16.6%, 33.3% and 49.9% across these three mutations were due to carrier statuses in GCDH in another sample which was confirmed. This response pattern was followed across seven other homozygous variants in mixing experiments (data not shown) with many of the observed proportion centered at ˜33.33% as expected.

### DETAILED DESCRIPTION

While various embodiments of the invention have been shown and described herein, it will be obvious to those skilled in the art that such embodiments are provided by way of example only. Numerous variations, changes, and substitutions can occur to those skilled in the art without departing from the invention. It should be understood that various alternatives to the embodiments of the invention described herein can be employed.

As used herein and in the appended claims, the singular forms “a”, “an”, and “the” include plural referents unless the context clearly dictates otherwise. Thus, for example, reference to “a device” includes a plurality of such devices known to those skilled in the art, and so forth.

Unless otherwise indicated, open terms for example “contain,” “containing,” “include,” “including,” and the like mean comprising.

The term “nucleic acid,” as used herein, generally refers to a polymeric form of nucleotides of any length, either ribonucleotides, deoxyribonucleotides or peptide nucleic acids (PNAs) that comprise purine and pyrimidine bases, or other natural, chemically or biochemically modified, non-natural, or derivatized nucleotide bases. A nucleic acid can refer to a polynucleotide. The backbone of the polynucleotide can comprise sugars and phosphate groups, as can be found in ribonucleic acid (RNA) or deoxyribonucleic acid (DNA), or modified or substituted sugar or phosphate groups. A polynucleotide can comprise modified nucleotides, such as methylated nucleotides and nucleotide analogs. The sequence of nucleotides can be interrupted by non-nucleotide components. Thus the terms nucleoside, nucleotide, deoxynucleoside and deoxynucleotide can generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. These analogs can be derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. The nucleic acid molecule can be a DNA molecule. The nucleic acid molecule can be an RNA molecule.

The term “neonatal”, as used herein, generally refer to things of or relating to a newborn.

The terms “variant” and “derivative,” as used herein in the context of a nucleic acid molecule, generally refer to a nucleic acid molecule comprising a polymorphism. Such terms can also refer to a nucleic acid product that is produced from one or more assays conducted on the nucleic acid molecule. For example, a fragmented nucleic acid molecule, hybridized nucleic acid molecule (e.g., capture probe hybridized nucleic acid molecule, bead bound nucleic acid molecule), amplified nucleic acid molecule, isolated nucleic acid molecule, eluted nucleic acid molecule, and enriched nucleic acid molecule are variants or derivatives of the nucleic acid molecule.

Where a range of values is provided, it is understood that each intervening value between the upper and lower limits of that range, to the tenth of the unit of the lower limit, unless the context clearly dictates otherwise, is also specifically disclosed. Each smaller range between any stated value or intervening value in a stated range, and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order which is logically possible.

### Overview

Of the approximately 4,000 single-gene disorders (Mendelian diseases) with a known molecular basis, a significant fraction can manifest symptoms during the newborn period. Newborn screening (NBS) programs can administer an infant's first biochemical screening test from a dried blood spot (DBS) specimen for 30 to 50 severe genetic disorders for which public health interventions exist, and thus these programs can be successful in preventing mortality or life-long debilitation. However, positive results can require complex second-tier confirmation to address false-positive results. For neonates with genetic disorders, a rapid diagnosis of newborn diseases can make the difference between life and death and reduce length of stay in the neonatal intensive care unit (NICU). However, in modern medical practice, acutely ill newborns can be stabilized in the NICU and discharged without a genetic diagnosis. The burden of genetic disorders is estimated at upwards of 25% of inpatient admissions in the newborn and pediatric population. Genetic testing can be performed gene by gene, based on available clinical indications and family histories, with each test conducted serially and costing thousands of dollars. With the advent of next-generation sequencing (NGS), large panels of genes can now be scanned together rapidly at a lower cost and with the added promise of reduced length of stay and better outcomes.

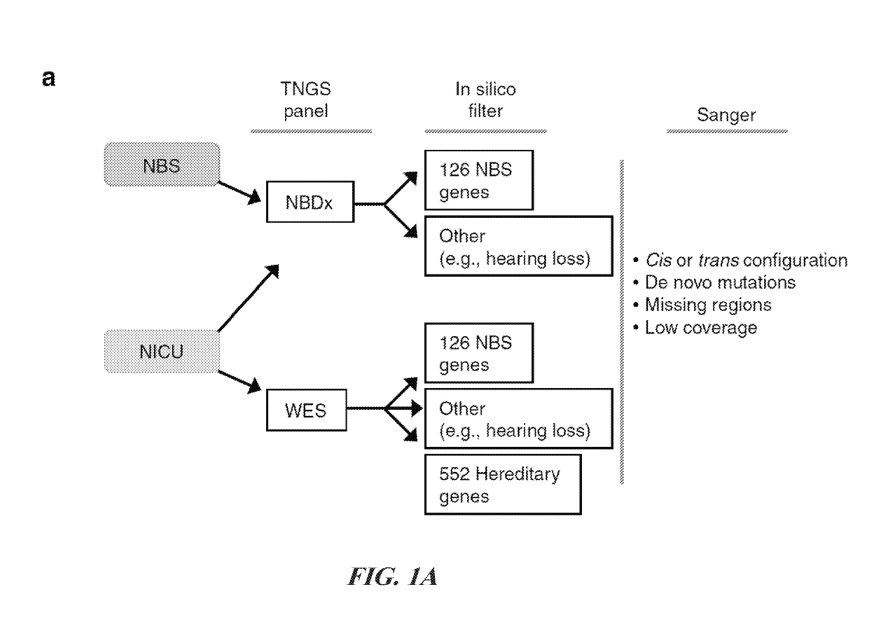

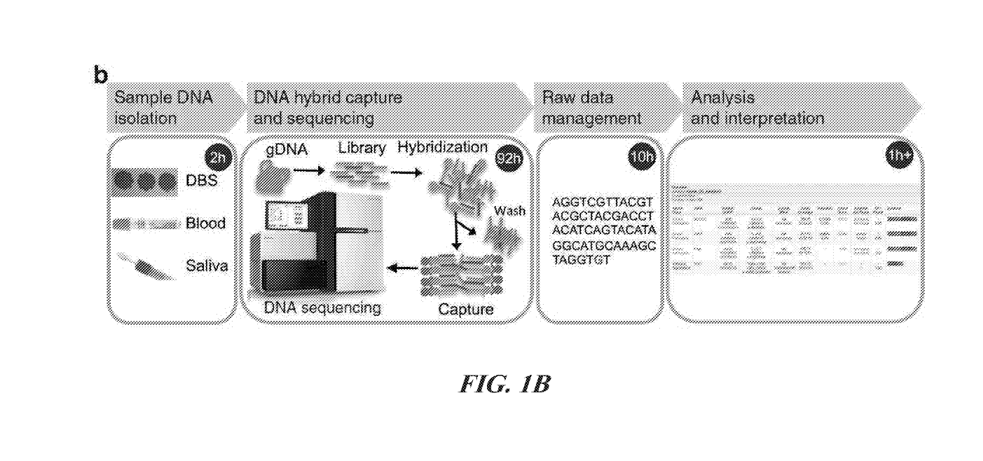

The methods and systems described herein can utilize a targeted next-generation sequencing (TNGS) assay which can cost-effectively addresses second-tier and diagnostic testing of newborns (FIG. 1A) by selectively sequencing genomic regions of interest, e.g., coding exons, by enrichment in a physical DNA capture step (FIG. 1B). TNGS can be re-purposed to also provide comprehensive coverage of elements such as introns. In many situations, the indicated symptoms can guide a focused investigation of specific disease genes (in silico gene filter; FIG. 1A). This can have the advantages of a rapid test, lower cost of interpretation, and avoidance of delays encountered with serial single-gene testing and ethical concerns of genome-scale NGS (surrounding unrecognized pathologic variants or unanticipated findings).

It can be impractical for newborns who have small total blood volumes to routinely provide the 2 to 10 ml of whole blood that can be requested for high-quality NGS services. Minimally invasive specimen types, such as DBS (wherein one spot is equivalent to 50 μl), if incorporated into the NGS workflow, can be more practical for newborns—avoiding stringent specimen handling and allowing accessibility in low-resource environments.

In addition, time to results can be critical for prompt treatment and management of life-threatening genetic disorders in newborns, NGS-based second-tier testing can have the advantage of improving performance of the primary biochemical NBS by reducing false positives (and parental anxiety), identifying de novo variants, and distinguishing genotypes associated with milder phenotypes (e.g., the mild R117H compared with the common pathological ΔF508 in cystic fibrosis). NGS second-tier DNA testing can also be useful especially for disorders such as cystinosis (OMIM 219800) that are not readily detectable via biochemistry.

The methods and systems disclosed herein can provide a fast-turnaround, minimally invasive, and cost-effective clinical sequencing and reporting for newborns. For purposes of context and explanation only, an example that incorporates the disclosed methods in the context of sequence variants associated with genetic disorders responsible for common phenotypes in the neonate is discussed. However, it should be understood that aspects of the disclosed methods described herein can be utilized in other systems and/or contexts, including other newborn genetic conditions.

A tiered approach can be used to identify genetic disorders in newborns. A newborn can first undergo NBS testing. Asymptomatic newborns who are identified as being at risk for or predisposed to disorders by NIBS can receive confirmation with second-tier testing (biochemical or genetic) on a repeat sample obtained from the patient in question. However, the genetic etiology, delayed onset, and/or “milder phenotype” can be missed. Symptomatic newborns, such as those admitted to a NICU, undergo an initial clinical assessment and sequential diagnostic testing to “rule out” causation; these can require nomination based on history or clinical opinions, thus limiting the diagnostic rate and efficiency. Because blood draws can be of concern in newborns, a single multigene sequencing panel can be used to minimize sequential analysis and avoid delayed diagnosis.

The approach of using gene panels and in silico filters can provide a systematic parallel or iterative review of symptom(s) and diseases from a molecular standpoint by providing information on the exact genes, their variant(s), and associated future risks (for family planning because of parental carrier status). In some cases, the burden of disease mutations and their combinations on phenotype or effect of cumulative mutations in genetic pathways that can act synergistically can not clearly be monitored by NBS or single-gene sequencing for newborn diseases. As an example, for a limited in silico filter size of 126 genes and 36 cases studied here, there were 19 incidental carrier mutations that were previously described in the Amish and Mennonite populations (Table 1 and Table 2), indicating that such information can help in identifying subclinical traits and reproductive planning.

TABLE 1
Concordance of called variants from blinded NBDx samples with a priori Sanger sequencing

| | | | | | | | Called | Requiring |
|---|---|---|---|---|---|---|---|---|
| | | | | | | | by | clinical |
| | | Transcript | Protein | Genomic | | | filters | pheno- |
| Sample | Gene | variant | variant | location | Zyg | Type | only | type |
| S1 | IL7R | c.2T>G | p.Met1Arg | g.5:35857081 | Hom | Nonsynonymous | Yes | |
| S3 | BTD | c.1459T>C | p.Trp487Arg | g.3:15686822 | Hom | Nonsynonymous | Yes | |
| S4^{a} | CYP11B1 | c.1343G>A | p.Arg448His | g.8:143956428 | Hom | Nonsynonymous | Yes | |
| S5^{a} | PAH | c.782G>A | p.Arg261Gln | g.12:103246653 | Het | Nonsynonymous | Yes | |
| | PAH | c.284_286del | p.Ile95del | g.12:103288579 | Het | Nonframeshift | Yes | |
| | | | | | | deletion | | |
| S6 | ACADM | c.985A>G | p.Lys329Glu | g.1:76226846 | Hom | Nonsynonymous | Yes | |
| S7 | CFTR | c.1521_152 | p.Phe508del | g.7:117199645 | Hom | Nonframeshift | No | Yes^{b} |
| | | 3del | | | | deletion | | |
| S9 | MTHFR | c.1129C>T | p.Arg377Cys | g.1:11854823 | Hom | Nonsynonymous | Yes | |
| S10^{a} | GALT | c.563A>G | p.Gln188Arg | g.9:34648167 | Hom | Nonsynonymous | Yes | |
| S11 | GCDH | c.1262C>T | p.A1a421Val | g.19:13010300 | Hom | Nonsynonymous | Yes | |
| 4963 | GCDH | c.1262C>T | p.A1a421Val | g.19:13010300 | Het | Nonsynonymous | Yes | |
| | GCDH | c.219delC | p.Thr73fs | g.19:13002735 | Het | Frameshift | | |
| | | | | | | deletion | | |
| 6810^{a} | GCDH | c.395 G>A | p.Arg132Gln | g.19:13004357 | Het | Nonsynonymous | No | Yes^{c} |
| | GCDH | c.877 G>A | p.Ala293Thr | g.19:1313007748 | Het | Nonsynonymous | | |
| 7066^{a} | GCDH | c.680 G>C | p.Arg227Pro | g.19:13007063 | Het | Nonsynonymous | Yes | |
| | GCDH | c.127 + G>A | | g.19:13002337 | Het | Splice site | | |
| 7241 | HPD | c.85 G>A | p.Ala29Thr | g.12:122295671 | Hom | Nonsynonymous | Yes | |
| 7656^{a} | GCDH | c.383 G>A | p.Arg128Gln | g.19:13004345 | Het | Nonsynonymous | Yes | |
| | GCDH | c.1060 G>A | p.Gly354Ser | g.19:13008220 | Het | Nonsynonymous | | |
| 7901 | GCDH | c.262 C>T | p.Arg88Cys | g.19:13002779 | Het | Nonsynonymous | Yes | |
| | GCDH | c.1262 C>T | p.Ala421Val | g.19:13010300 | Het | Nonsynonymous | | |
| 7912 | GCDH | c.344 G>A | p.Cys115Tyr | g.19:13004306 | Het | Nonsynonymous | Yes | |
| | GCDH | c.1063 C>T | p.Arg355Cys | g.19:13008223 | Het | Nonsynonymous | | |
| 9226 | ACADM | c.985 A>G | p.Lys329Glu | g.1:76226846 | Het | Nonsynonymous | No | Yes^{d,e} |
| | ACADM | c.287-30 | | g.1:76199183 | Het | Intronic | | |
| | | A>G | | | | | | |
| 10241 | GCDH | c.190 G>C | p.Glu646Gln | g.19:13002707 | Het | Nonsynonymous | Yes | |
| | GCDH | c.281 G>T | p.Arg94Leu | g.19:13002939 | Het | Nonsynonymous | | |
| 10642^{a} | GCDH | c.1093 G>A | p.Glu365Lys | g.19:13008527 | Het | Nonsynonymous | Yes | |
| | GCDH | c.1240G>A | p.Glu414Lys | g.19:13008674 | Het | Nonsynonymous | | |
| 13925 | c7orf10 | c.895C>T | p.Arg299Trp | g.7:40498796 | Hom | Nonsynonymous | Yes | |
| 14691 | DBT | c.634 C>T | p.Gln212* | g.1:100681677 | Het | Stop gained | No | Yes^{d,e} |
| | DBT | c.1209 + 5 | splice site | g.1:100671996 | Het | Splice site | | |
| | | G>C | | | | | | |
| 16622 | ACADM | c.985 A>G | p.Lys329Glu | g.1:76226846 | Het | Nonsynonymous | No | Yes^{d,e} |
| | ACADM | c.600-18 | intronic | g.1:76211473 | Het | intronic | | |
| | | G>A | | | | | | |
| 18087^{a} | BCKDHB | c.548 G>C | p.Arg183Pro | g.6:80878662 | Het | Nonsynonymous | Yes | |
| | BCKDHB | c.583_584ins | p.Tyr195fs | g.6:80878697 | Het | Frameshift | | |
| | | | | | | insertion | | |
| 19283^{a} | GALT | c.563A>G | p.Gln188Arg | g.9:34648167 | Hom | Nonsynonymous | Yes | |
| 21901 | CYP21A2 | NR | | g. | | | No | No^{f} |
| 22785^{a} | GCDH | c.1198 G>A | p.Val400Met | g.19:13008632 | Het | Nonsynonymous | No | Yes^{c} |
| | GCDH | c.1213 A>G | p.Met405Val | g.19:13008647 | Het | Nonsynonymous | | |
| 23275^{a} | BTD | c.1368 A>C | p.Gln456His | g.3:15686731 | Het | Nonsynonymous | N/A | |
| 23279^{a} | BTD | c.1330G>C | p.Asp444His | g.3:15686693 | Hom | Nonsynonymous | Yes | |
| 25875 | HPD | c.479 A>G | p.Tyr160Cys | g.12:122287632 | Het | Nonsynonymous | Yes | |
| | HPD | c.1005 C>G | p.Ile335Met | g.12:122277904 | Het | Nonsynonymous | | |
| 26607 | GCDH | c.442 G>A | p.Val148Ile | g.19:13004404 | Het | Nonsynonymous | Yes | |
| | GCDH | c.452 C>G | p.Pro151Arg | g.19:13004414 | Het | Nonsynonymous | | |
| 27244^{a} | CYP21A2 | NR | | g. | | | No | No^{f} |
| 27527 | BCKDHA | c.649 G>C | p.Val217Leu | g.19:41928071 | Het | Nonsynonymous | Yes | |
| | BCKDHA | c.659 C>T | p.Ala220Val | g.19:41928081 | Het | Nonsynonymous | | |
| 29351 | MCCC2 | c.295 G>C | p.Glu99Gln | g.5:70895499 | Hom | Nonsynonymous | Yes | |
| 30221^{a} | HPD | c.479 A>G | p.Tyr160Cys | g.12:122287632 | Het | Nonsynonymous | N/A | |
| 31206^{a} | MCCC2 | c.517_518ins | p.Ser173fs | g.5:70900187 | Hom | Frameshift | No | Yes^{b} |
| | | | | | | insertion | | |
| 31908 | HSD3B2 | c.35 G>A | p.Gly12Glu | g.1:119958077 | Hom | Nonsynonymous | Yes | |

Variant calls for causal mutations and carrier statuses in blinded samples previously Sanger sequenced at the Clinic for Special Children. Samples are further marked for any requirements of de-blinding for clinical characteristics prior to identification from the targeted next-generation sequencing pipeline. Also noted are discrepancies, potential false positives, and other issues for identification.
^{a}Sample has at least one carrier mutation in the 126 NBS genes. ^{b}Misannotated during first filtering. ^{c}Could not distinguish from another gene with two heterozygous variants. ^{d}False positive in absence of clinical description elentronic filter applied after clinical information given. ^{f}CYP21A2 not tiled on panel (due to pseudogene).

TABLE 2
Coverage for NBDx Samples per Tiled Region.

| Gene | Tile Coordinates | S1 | S3 | S4 | S6 | S9 | S11 | 10241 | 10642 | 13925 | 14691 | 16622 | 29351 |
|---|---|---|---|---|---|---|---|---|---|---|---|---|---|
| MTHFR | 1:11864520-11866198 | 0.97 | 0.94 | 0.98 | 0.98 | 0.98 | 0.99 | 0.96 | 1 | 1 | 0.99 | 0.97 | 0.97 |
| DBT | 1:100661510-100661996 | 0.99 | 0.93 | 0.99 | 0.98 | 0.99 | 0.99 | 0.99 | 0.99 | 0.98 | 0.99 | 0.99 | 0.99 |
| HMGCL | 1:24151797-24151978 | 1 | 0.92 | 1 | 1 | 0.98 | 1 | 1 | 1 | 1 | 1 | 1 | 1 |
| MTR | 1:236958539-236959066 | 1 | 1 | 0.97 | 1 | 1 | 1 | 1 | 1 | 1 | 1 | 1 | 1 |
| CPT2 | 1:53662054-53662799 | 0.88 | 0.89 | 0.9 | 0.91 | 0.94 | 0.93 | 0.87 | 0.9 | 0.86 | 0.92 | 0.86 | 0.91 |
| GALE | 1:24122042-24122533 | 0.98 | 1 | 1 | 1 | 1 | 1 | 1 | 1 | 1 | 1 | 1 | 1 |
| PTPRC | 1:198663335-198663467 | 0.94 | 1 | 1 | 1 | 1 | 1 | 1 | 1 | 1 | 1 | 1 | 1 |
| SPR | 2:73114470-73114892 | 0.45 | 0.57 | 0.47 | 0.57 | 0.55 | 0.69 | 0.45 | 0.54 | 0.71 | 0.39 | 0.58 | 0.42 |
| ACADL | 2:211089884-211090234 | 0.97 | 0.88 | 0.9 | 0.89 | 0.99 | 1 | 0.99 | 0.99 | 1 | 0.99 | 1 | 0.96 |
| ZAP70 | 2:98340427-98340935 | 0.97 | 0.96 | 1 | 0.98 | 1 | 1 | 0.97 | 1 | 0.99 | 0.96 | 1 | 0.92 |
| ZAP70 | 2:98350818-98351206 | 1 | 0 | 0 | 0 | 0 | 1 | 0 | 0 | 1 | 0 | 0 | 0 |
| MCCC1 | 3:182817133-182817397 | 0.99 | 1 | 1 | 1 | 1 | 1 | 1 | 1 | 1 | 1 | 1 | 1 |
| PCCB | 3:136009520-136009719 | 0.97 | 0.98 | 0.98 | 0.98 | 1 | 0.99 | 0.98 | 0.97 | 0.98 | 0.97 | 0.97 | 0.98 |
| PCCB | 3:136018814-136018954 | 0.99 | 1 | 0.89 | 0.92 | 0.91 | 0.96 | 1 | 0.87 | 1 | 0.82 | 1 | 1 |
| PCCB | 3:136019019-136021390 | 1 | 1 | 1 | 1 | 1 | 1 | 1 | 0.83 | 1 | 0.83 | 1 | 1 |
| PCCB | 3:136021575-136021659 | 1 | 1 | 1 | 1 | 1 | 1 | 1 | 0 | 1 | 0 | 1 | 1 |
| PCCB | 3:136021890-136022434 | 1 | 1 | 1 | 1 | 1 | 1 | 1 | 0 | 1 | 0 | 1 | 1 |
| PCCB | 3:136022735-136022818 | 1 | 1 | 1 | 1 | 1 | 1 | 1 | 0 | 1 | 0 | 1 | 1 |
| PCCB | 3:136022845-136023003 | 1 | 1 | 1 | 1 | 1 | 1 | 1 | 0 | 1 | 0 | 1 | 1 |
| PCCB | 3:136023810-136023949 | 1 | 1 | 1 | 1 | 1 | 1 | 1 | 0 | 1 | 0 | 1 | 1 |
| PCCB | 3:136024215-136024295 | 1 | 1 | 1 | 1 | 1 | 1 | 1 | 0 | 1 | 0 | 1 | 1 |
| PCCB | 3:136024605-136025351 | 1 | 1 | 1 | 1 | 1 | 1 | 1 | 0 | 1 | 0 | 1 | 1 |
| PCCB | 3:136025374-136026479 | 1 | 1 | 1 | 1 | 1 | 1 | 1 | 0.25 | 1 | 0.25 | 1 | 1 |
| SLC25A20 | 3:48936076-48936454 | 1 | 0.94 | 0.96 | 1 | 1 | 1 | 1 | 1 | 0.92 | 0.98 | 0.99 | 1 |
| BTD | 3:15651403-15653172 | 1 | 0.99 | 0.99 | 1 | 1 | 1 | 1 | 1 | 1 | 1 | 1 | 1 |
| QDPR | 4:17513526-17513373 | 0.24 | 0.48 | 0.44 | 0.46 | 0.34 | 0.55 | 0.13 | 0.41 | 0.39 | 0.46 | 0.56 | 0.32 |
| HADH | 4:108910819-108911248 | 0.99 | 0.87 | 0.94 | 0.87 | 0.9 | 0.97 | 0.9 | 0.9 | 0.89 | 0.93 | 0.86 | 1 |
| MTRR | 5:7891414-7891589 | 1 | 1 | 1 | 1 | 1 | 0.95 | 0.97 | 1 | 0.95 | 0.97 | 1 | 1 |
| M7RR | 5:7891414-7891589 | 1 | 1 | 1 | 1 | 1 | 0.95 | 0.97 | 1 | 0.95 | 0.97 | 1 | 1 |
| MCCC2 | 5:70883066-70883409 | 1 | 0.94 | 1 | 1 | 1 | 1 | 1 | 1 | 1 | 1 | 1 | 1 |
| BCKDHB | 6:80816302-80817017 | 1 | 0.99 | 1 | 1 | 1 | 1 | 1 | 1 | 1 | 1 | 1 | 1 |
| BCKDHB | 6:81033777-81036331 | 1 | 0.99 | 1 | 1 | 1 | 1 | 1 | 1 | 1 | 1 | 1 | 1 |
| BCKDHB | 6:81045702-81046217 | 0.96 | 0.98 | 1 | 0.97 | 1 | 0.98 | 1 | 0.97 | 0.97 | 0.97 | 1 | 0.99 |
| BCKDHB | 6:81046217-81046674 | 1 | 0.99 | 1 | 0.98 | 1 | 1 | 1 | 0.99 | 0.99 | 0.99 | 1 | 1 |
| LMBRD1 | 6:70506656-70507090 | 0.94 | 0.78 | 0.75 | 0.84 | 0.98 | 0.91 | 0.9 | 0.95 | 0.89 | 0.92 | 0.84 | 0.88 |
| ASL | 7:65540724-65541113 | 0.65 | 0.62 | 0.66 | 0.65 | 0.57 | 0.97 | 0.65 | 0.65 | 0.7 | 0.6 | 0.54 | 0.65 |
| SLC25A13 | 7:95951205-95951485 | 1 | 1 | 0.96 | 1 | 0.96 | 1 | 0.99 | 0.93 | 1 | 1 | 0.94 | 0.96 |
| C7orf10 | 7:40174523-40174749 | 0.96 | 1 | 1 | 0.98 | 0.81 | 0.87 | 0.88 | 0.98 | 0.97 | 0.95 | 0.74 | 0.82 |
| CFTR | 7:117144820-117145518 | 1 | 1 | 1 | 1 | 1 | 1 | 1 | 1 | 0.99 | 1 | 1 | 1 |
| CFTR | 7117252110-117252297 | 1 | 1 | 1 | 1 | 1 | 1 | 1 | 1 | 0.98 | 1 | 1 | 1 |
| CFTR | 7:117254070-117255017 | 1 | 1 | 1 | 1 | 1 | 1 | 1 | 1 | 1 | 1 | 1 | 0.99 |
| OPLAH | 8:145106116-145106394 | 0.95 | 0.96 | 0.94 | 0.93 | 0.95 | 0.99 | 0.95 | 0.95 | 0.98 | 1 | 0.95 | 0.97 |
| OPLAH | 8:145106770-145107262 | 0.86 | 0.95 | 0.62 | 0.94 | 0.72 | 0.99 | 0.83 | 0.85 | 0.94 | 0.94 | 0.87 | 0.85 |
| OPLAH | 8:145107310-145107532 | 1 | 1 | 0.83 | 1 | 0.89 | 1 | 0.84 | 0.93 | 1 | 1 | 0.9 | 1 |
| OPLAH | 8:145109885-145110133 | 0.86 | 0.88 | 0.89 | 0.87 | 0.86 | 0.91 | 0.82 | 0.9 | 0.89 | 0.9 | 0.88 | 0.89 |
| OPLAH | 8:145111479-145111675 | 1 | 1 | 1 | 0.89 | 1 | 1 | 1 | 1 | 1 | 1 | 1 | 1 |
| OPLAH | 8:145111801-145112055 | 1 | 1 | 0.95 | 1 | 1 | 1 | 1 | 1 | 1 | 0.99 | 1 | 1 |
| OPLAH | 8:145113095-145113335 | 1 | 1 | 0.98 | 1 | 0.97 | 1 | 1 | 1 | 1 | 0.97 | 1 | 1 |
| OPLAH | 8:145113352-145114022 | 1 | 1 | 0.99 | 1 | 1 | 1 | 1 | 1 | 1 | 0.95 | 1 | 1 |
| OPLAH | 8:145115468-145115649 | 0 | 0 | 0.52 | 0.03 | 0.33 | 0.65 | 0 | 0.58 | 0.55 | 0 | 0.12 | 0.45 |
| ASS1 | 9:133320065-133320381 | 0.47 | 0.68 | 0.53 | 0.3 | 0.69 | 0.01 | 0 | 0.74 | 0.25 | 0.42 | 0.26 | 0.12 |
| AUH | 9:94123858-94124219 | 0.94 | 0.95 | 0.78 | 0.84 | 1 | 0.93 | 0.82 | 0.99 | 0.96 | 0.95 | 0.96 | 0.92 |
| MAT1A | 1082031529-82033657 | 1 | 1 | 1 | 1 | 1 | 1 | 1 | 0.99 | 1 | 1 | 1 | 1 |
| PCBD1 | 10:72648238-72648560 | 0.76 | 0.69 | 0.88 | 0.68 | 0.85 | 0.72 | 0.66 | 0.69 | 0.8 | 0.66 | 0.68 | 0.67 |
| ACADSB | 10:124810516- | 0.99 | 0.99 | 0.99 | 0.98 | 1 | 0.98 | 0.98 | 0.99 | 0.99 | 0.98 | 0.99 | 1 |
| | 124810709 | | | | | | | | | | | | |
| ACADSB | 10:124810516- | 0.99 | 0.99 | 0.99 | 0.98 | 1 | 0.98 | 0.98 | 0.99 | 0.99 | 0.98 | 0.99 | 1 |
| | 124810709 | | | | | | | | | | | | |
| PTS | 1.1:11209847037- | 1 | 0.92 | 0.88 | 0.79 | 0.78 | 0.98 | 0.69 | 0.88 | 0.96 | 0.69 | 1 | 0.73 |
| | 1120972 | | | | | | | | | | | | |
| ACAT1 | 11:107992209- | 0.99 | 1 | 1 | 0.99 | 0.98 | 1 | 1 | 1 | 1 | 1 | 1 | 1 |
| | 107992437 | | | | | | | | | | | | |
| CPT1A | 11:68609196-68609284 | 0 | 0.24 | 0 | 0.2 | 0.36 | 0.11 | 0 | 0.82 | 0 | 0 | 0 | 0.39 |
| CPT1A | 11:68609286-68609429 | 0 | 0 | 0.52 | 0.38 | 0.36 | 0.27 | 0 | 0.54 | 0.02 | 0 | 0 | 0 |
| PAH | 12:103240377- | 1 | 1 | 1 | 1 | 1 | 1 | 1 | 1 | 1 | 1 | 0.99 | 1 |
| | 103242058 | | | | | | | | | | | | |
| PAH | 12103297172- | 1 | 1 | 1 | 1 | 1 | 0.99 | 1 | 0.99 | 1 | 1 | 0.99 | 1 |
| | 103298744 | | | | | | | | | | | | |
| MMAB | 12:109998800- | 1 | 1 | 1 | 1 | 1 | 1 | 1 | 1 | 0.99 | 1 | 1 | 1 |
| | 109998951 | | | | | | | | | | | | |
| MMAB | 12:110011104- | 1 | 1 | 1 | 0.97 | 1 | 1 | 1 | 1 | 1 | 1 | 1 | 1 |
| | 110011390 | | | | | | | | | | | | |
| ACADS | 12:121163523- | 0.96 | 0.93 | 1 | 0.8 | 1 | 1 | 0.81 | 0.83 | 0.8 | 0.87 | 0.9 | 0.87 |
| | 121163763 | | | | | | | | | | | | |
| SLC25A15 | 13:41363505-41363803 | 0.45 | 0.43 | 0.69 | 0.67 | 0.72 | 0.63 | 0.5 | 0.64 | 0.54 | 0.48 | 0.67 | 0.42 |
| PCCA | 13:100814781- | 1 | 1 | 0.99 | 1 | 1 | 1 | 1 | 1 | 1 | 0.99 | 1 | 1 |
| | 100816934 | | | | | | | | | | | | |
| PCCA | 13:100936078- | 1 | 1 | 1 | 0.68 | 1 | 1 | 0.53 | 0.62 | 0.99 | 0.78 | 1 | 0.23 |
| | 100936155 | | | | | | | | | | | | |
| PCCA | 13:100977371- | 1 | 1 | 0.93 | 0.92 | 1 | 0.95 | 0.93 | 1 | 1 | 0.95 | 1 | 1 |
| | 100977990 | | | | | | | | | | | | |
| PCCA | 13:101080597- | 1 | 1 | 1 | 0.99 | 1 | 1 | 1 | 1 | 1 | 1 | 1 | 1 |
| | 101081855 | | | | | | | | | | | | |
| PCCA | 13:101092457- | 0.99 | 0.97 | 1 | 0.99 | 0.97 | 0.97 | 1 | 1 | 1 | 0.98 | 1 | 1 |
| | 101092811 | | | | | | | | | | | | |
| GCH1 | 14:55368992-55369573 | 1 | 0.96 | 0.98 | 0.94 | 1 | 0.97 | 0.96 | 0.99 | 0.98 | 0.99 | 1 | 1 |
| FAH | 15:80445186-80445512 | 0.95 | 0.99 | 1 | 1 | 0.98 | 1 | 0.94 | 1 | 0.99 | 0.98 | 1 | 1 |
| FAH | 15:80478425-80478939 | 1 | 1 | 1 | 0.98 | 1 | 1 | 1 | 1 | 1 | 1 | 1 | 1 |
| IVD | 15:40697639-40698193 | 0.95 | 1 | 1 | 1 | 1 | 1 | 1 | 1 | 1 | 1 | 1 | 1 |
| IVD | 15:40713477-40713546 | 1 | 1 | 1 | 0.94 | 1 | 1 | 1 | 1 | 1 | 1 | 1 | 1 |
| MLYCD | 16:83932683-83933299 | 0.62 | 0.38 | 0.67 | 0.74 | 0.35 | 0.73 | 0.56 | 0.8 | 0.6 | 0.65 | 0.36 | 0.5 |
| GALK1 | 17:73761006-73761315 | 0.77 | 0.77 | 0.75 | 0.81 | 0.76 | 0.87 | 0.79 | 0.82 | 0.82 | 0.78 | 0.85 | 0.75 |
| BCKDHA | 19:41914177-41914539 | 1 | 1 | 0.99 | 1 | 1 | 1 | 1 | 1 | 1 | 1 | 1 | 1 |
| OPA3 | 19:46056217-46057203 | 0.99 | 1 | 0.93 | 0.96 | 0.9 | 0.89 | 0.98 | 0.94 | 0.94 | 0.92 | 0.99 | 0.95 |
| ETFB | 19:51857352-51858026 | 0.99 | 1 | 1 | 1 | 1 | 1 | 1 | 1 | 1 | 1 | 1 | 1 |
| ETFB | 19:51858027-51858122 | 0.9 | 0.8 | 0.99 | 1 | 0.98 | 1 | 0.95 | 1 | 1 | 0.6 | 0.84 | 0.91 |
| ETFB | 19:51869477-51869702 | 1 | 1 | 1 | 1 | 1 | 1 | 1 | 1 | 1 | 0.96 | 1 | 1 |
| JAK3 | 19:17940870-17941057 | 1 | 1 | 1 | 0.95 | 1 | 1 | 1 | 1 | 1 | 1 | 1 | 1 |
| JAK3 | 19:17942434-17942644 | 1 | 0.96 | 1 | 1 | 0.96 | 1 | 1 | 1 | 1 | 1 | 1 | 1 |
| JAK3 | 19:17953078-17953446 | 0.95 | 1 | 0.94 | 0.98 | 1 | 1 | 0.88 | 0.94 | 1 | 0.92 | 0.99 | 0.96 |
| ADA | 20:43280168-43280401 | 0.86 | 0.85 | 0.79 | 0.83 | 0.85 | 0.86 | 0.83 | 0.77 | 0.76 | 0.76 | 0.79 | 0.81 |
| CBS | 21:44473319-44474129 | 1 | 1 | 0.98 | 1 | 1 | 1 | 0.99 | 1 | 0.99 | 1 | 1 | 1 |
| CBS | 21:44485447-44485851 | 0.99 | 1 | 1 | 0.96 | 0.95 | 1 | 1 | 1 | 0.97 | 1 | 1 | 1 |
| CBS | 21:44495842-44496069 | 0.87 | 0.88 | 0.9 | 0.82 | 0.91 | 0.92 | 0.93 | 0.86 | 0.8 | 0.87 | 0.82 | 0.91 |
| HLCS | 21:38338695-38338991 | 0 | 0 | 0 | 0 | 0 | 0 | 0 | 0 | 0 | 0 | 0 | 0 |
| HLCS | 21:38353032-38353289 | 0.72 | 0.9 | 0.96 | 0.95 | 1 | 0.98 | 1 | 1 | 0.98 | 1 | 0.81 | 1 |
| HLCS | 21:38362405-38362589 | 1 | 1 | 0.39 | 0.99 | 0.88 | 1 | 0.79 | 0.92 | 1 | 0.88 | 0.98 | 1 |
| G6PD | X:153760014-153760329 | 1 | 0.79 | 0.99 | 1 | 0.86 | 1 | 1 | 1 | 1 | 1 | 1 | 1 |
| G6PD | X:153774956-153775263 | 0.48 | 0.43 | 0.27 | 0.34 | 0.39 | 0.46 | 0.44 | 0.46 | 0.41 | 0.27 | 0.2 | 0.45 |
| IL2RG | X:70327342-70327808 | 1 | 0.99 | 1 | 1 | 1 | 1 | 1 | 1 | 1 | 1 | 1 | 1 |

In the context of neonatal care, genomic tests like NBDx and WES can, as part of a testing menu, precisely inform in one test what the prenatal tests, ultrasounds, amniocentesis, and NBS test sometimes cannot. Diagnosis can be helpful, even when no therapies are available, and can allow parents of affected children to be informed about their care up-front and receive genetic counseling regarding the risk for future pregnancies.

The disclosed comprehensive rapid test workflow for second-tier NBS testing and high-risk diagnosis of newborns for multiple genetic disorders can approach a 2- to 3-day turnaround for newborns to avoid medical sequelae. In some cases, the test processes a single sample at a time. In some cases, the test parallel-processes 2 to 96 samples per lane, for example, 2, 3, 4, 5, 6, 7, 8, 9, 10, 11, 12, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 samples per lane. In some cases, the test is completed in less than 100 hours, for example, in less than 10, 11, 12, 13, 14, 15, 16, 17, 18, 19, 20, 25, 30, 35, 40, 45, 50, 55, 60, 65, 70, 75, 80, 85, 90, 95, or 100 hours. In one example, the test can be parallel-processed for 8 to 20 samples per lane and completed in 105 hours (approximately 4.5 days); and several approaches to reduce turnaround time can be promising, such as alternate library preparation and reduced hybridization time. In cases in which mutations are suspected to be in trans, additional follow-up testing can be required. Provided herein are improved methods and systems for the minimally invasive isolation of high-quality double-stranded (dsDNA) from DBS and small blood volumes (25-50 μl) in sufficient amounts for TNGS. Adoption of DBS-based NGS testing can significantly reduce the burden of using more expensive lavender (purple) top tubes for blood collection, which can add to special handling, shipping, and storage costs. Moving an NGS test to DBS can enable widespread utility using centralized NGS testing facilities. When available, cord blood can be used as an alternative minimally invasive biological specimen source for TNGS, or dried on a card, similar to current DBS, for simplified transport. In some cases, dried blood collected on cellulose do not have clotting agents like heparin or EDTA, therefore subsequent extraction of DNA is quite difficult. Treatments of blood with such agents can have variable effects on DNA extraction as can be noted in downstream utilities.

A new approach of isolating DNA from blood spots, specifically from newborns, using extraction methods that do not denature the DNA has been developed. The DNA in double stranded format can be used for subsequent application in next generation sequencing workflow because in many such applications a synthetic adapter is ligated for sample barcoding, strand barcoding, transposition by transposases (e.g. Nextera), methylation and/or DNA amplification. In some cases, isolating double stranded DNA can be performed when there is cellular heterogeneity. In some cases, isolating double stranded DNA can be performed because the variation in both strands is a hallmark of true variation which can be lost when using single stranded DNA. In other cases, isolating double stranded DNA can be performed when using single molecule sequencing methods.

When disease heterogeneity or multigene diseases are encountered during the newborn period (e.g., phenylketonuria, severe combined immunodeficiency disease, maple syrup urine disease, propionic acidemia, glutaric acidemia), a TNGS assay covering approximately 100 to 300 disease genes can be as cost-effective as Sanger sequencing test(s) for quickly confirming or “ruling out” clinical suspicion or false-positive results. The cost of NBDx can be significantly less than that of WES, and both tests can be expected to be similar in price range to diagnostic tests currently on the market and therefore can enable replacement of single-gene tests, as justified by delays and increased patient-management costs.

Performance benchmarks can be established to support direct clinical use similar to WGS newborn/pediatric testing of Mendelian diseases. In the NICU setting, either WES or NBDx adapted for minimal invasive sample size or rapid turnaround can assist in detecting mutations and diagnosing the critically ill, some of whom can have metabolic decompensation at birth. Even after NBS, cases of cystic fibrosis and metabolic conditions are routinely missed (false negatives) because of various causes, including biochemical cutoffs. NGS-based testing can improve sensitivity. In some cases, exon deletion, which is not covered in NBS, can be detected in maple syrup urine disease cases using NGS-based testing.

In some cases, the methods and systems (e.g., test) are preconfigured to include NGS to improve diagnosis and differential diagnosis, including CMV tests, mitochondrial and nuclear gene test panels.

In some cases, despite a classic disease-causing mutation, the phenotype can be absent. Phenotypic information as part of NBS or clinical diagnosis can improve genotype call. Thus, with the clinical phenotype description, single-nucleotide variations in exons, introns (up to 30 bp away from an exon), and indels can be used to improve the accuracy of disease detection. With phenotypic information, a heuristic variant- and disease-calling pipeline can be built and automated.

### Subjects

Often, the methods and systems are used on a subject. The subjects can be mammals or non-mammals. The subjects can be a mammal, such as, a human, non-human primate (e.g., apes, monkeys, chimpanzees), cat, dog, rabbit, goat, horse, cow, pig, rodent, mouse, SCID mouse, rat, guinea pig, or sheep. A subject can be a mother, father, brother, sister, aunt, uncle, cousin, grandparent, great-grandparent, great-great grandparent, niece, and/or nephew. A subject can be a family member and/or have family members. A subject can be a family member of another subject. A subject can be related by marriage to another subject. A subject can be a relative of another subject. A subject can be distantly related to another subject. A relative can be related by blood or by marriage.

A subject can be a fetus, newborn, infant, child, adolescent, teenager or adult. A fetus can be a prenatal human between the embryonic state and birth. For example, a fetus can be a prenatal human of at least 9, 10, 11, 12, 13, 14, 15, 16, 17, 18, 19, 20, 25, 30, 35, 40, or 45 weeks after fertilization and before the birth. A subject can be an infant within the first 12 months after birth, for example, within 1, 2, 3, 4, 5, 6, 7, 8, 9, 10, 11, or 12 months after birth. A subject can be a child below the age of 10 years, for example, 1, 2, 3, 4, 5, 6, 7, 8, 9 or 10 years old. A subject can be an adolescent or a teenager during the period from puberty to legal adulthood. For example, an adolescent or a teenager can be 10, 11, 12, 13, 14, 15, 16, 17, 18, 19, 20, or 21 years old. A subject can be an adult.

The subject can be a newborn, wherein the newborn is within the first 28 days after birth, for example within 1 hour, 3 hours, 6 hours, 12 hours, 1 day, 2 days, 3 days, 4 days, 5 days, 6 days, 7 days, 8 days, 9 days, 10 days, 12 days, 14 days, 16 days, 18 days, 20 days, 22 days, 24 days, 26 days, or 28 days after birth. In a typical case, the newborn is within the first 28 days after birth. In some cases, a newborn can be born prematurely, for example, prior to full gestation period, for example, less than 30, 31, 32, 33, 34, 35, 36, 37, 38, 39, or 40 weeks gestational age. In some cases, a newborn can be born after full gestation period, for example, more than 40, 41, 42, 43, 44, or 45 weeks gestational age. A subject can be a newborn that requires a period of stay, for example, at least 1 hour, 2 hours, 3 hours, 4 hours, 5 hours, 6 hours, 12 hours, 24 hours, 36 hours, 48 hours, 3 days, 4 days, 5 days, 6 days, 7 days, 2 weeks, 3 weeks, or 4 weeks at the neonatal intensive care unit (NICU).

The methods and systems can be used for detecting, predicting, screening and/or determining the presence, absence or predisposition of a genetic condition in a subject. The genetic condition can be caused by a genetic disorder. Determining the presence or absence of a genetic condition (e.g., a genetic disorder) can include determining the predisposition and/or susceptibility to the genetic condition. Determining the presence, absence or predisposition of a genetic condition (e.g., a genetic disorder) can also include determining the possibility of developing the genetic condition. In some cases, the genetic condition is a disease (e.g., genetic disease), a phenotype, a disorder (e.g., genetic disorder) and/or a pathogen (e.g., virus, bacterium, priori, fungus, or parasite). In some cases, the genetic condition is a hearing loss condition. In some cases, determining the presence, absence or predisposition of the hearing loss condition comprises determining the presence, absence or predisposition of a nucleic acid (e.g., DNA, RNA) sequence, a mitochondrial DNA sequence, or a pathogen genomic sequence. The sequence can be a whole genome sequence or a partial genome sequence. In some cases, the genetic disorder is a single gene disorder, which is the result of a single mutated gene. In some cases, the genetic disorder is a complex, multifactorial, or polygenic disorder, which is likely to be associated with the effects of multiple genes. The genetic condition in the subject can also be caused by a pathogenic disease (e.g. viral infection). For example, newborns infected by cytomegalovirus (CMV) can result in hearing loss in the newborn.

In some methods and systems, species variants or homologs of these genes can be used in a non-human animal model. Species variants can be the genes in different species having greatest sequence identity and similarity in functional properties to one another. Many of such human species variants genes can be listed in the Swiss-Prot database.

### Diseases and Disorders

In some instances, a subject of the disclosure can have a disease. In some instances, the subject can show symptoms of a disease but not be diagnosed with a disease. In some instances, the subject can have a disease but not know it or can be undiagnosed. Diseases can include, cancers (e.g., retinoblastoma, lung, skin, breast, pancreas, liver, colon), cutaneous disease (e.g., icthyosis, acne, glandular rosacea, rhinophyma, otophyma, metophyma, lupus, periorificial dermatitis, dermatitis, psoriasis, Blau syndrome, familial cold urticaria, Majeed syndrome, Muckle-Wells syndrome), endocrine diseases (e.g., adrenal disorders, adrenal hormone excess, diabetes, hypoglycemia, glucagonoma, goiter, hyperthyroidism, hypothyroidism, parathyroid disorders, pituitary gland disorders, sex hormone disorders, hermaphroditism), eye diseases (e.g., disorders of the eyelid, hordeolum, chalazion, disorders of the conjunctiva, conjunctivitis, disorders of the sclera, cornea, iris and ciliary body, scleritis, keratitis, Fuch's dystrophy, disorders of the lens, cataract, disorders of the choroid and retina, chorioretinal inflammation, retinitis, choroidal degeneration, retinal detachments, retinal vascular occlusions, glaucoma, disorders of the vitreous body and globe, disorders of the optic nerve and visual pathways, optic disc drusen, blindness), intestinal diseases, infectious diseases. In some instances, a subject can have a disorder. Disorders can include hearing disorders, muscle disorders, connective tissue disorders, genetic disorders, neurological disorders, voice disorders, vulvovaginal disorders, mental illness, autism disorders, eating disorders, mood disorders, and personality disorders.

In one example, the NBDxV1.0 gene panel includes 227 genes that relates to four categories of diseases: 1) Newborn Screening Disorder related (107 genes); 2) Expanded neonatal screening panel (19 genes); 3) Hearing loss, non-syndromic (84 genes); 4) Hypotonia, hepatosplenomegaly and failure to thrive (17 genes).

In another example, NBDxV1.1 gene panel includes 586 genes that relates to the following categories of diseases: Newborn Screening Disorders, Expanded Neonatal Screening, Neonatal Inborn Errors, Hearing Loss: non-syndromic, Hypoglycemia: HI, PHHI, Syndromes, Hypotonia, Neonatal Seizures, NS Microcephaly, Hyperbilirubinemia, Hepatosplenomegaly, Liver Failure, Respiratory Failure, Skeletal Dysplasia, Renal Dysplasia, Anemia, Neutropenia, Thrombophilia, Thrombocytopenia, Bleeding Diathesis, Cancer: RB, DICER, RET, ALK, Cutaneous: EB, ichthyosis, Hirschsprung's disease, Neonatal Abstinence, Pharmacogenomics (e.g. G6PD), Miscellaneous Syndromes: Noonan, Marfans, Holt-Oram, Wardenberg, and WAGR/Denys-Drasch. The list of genes in NBDxV1.1 gene panel is shown in Example 5, Table 14.

In another example, hypotonia can be symptomatic of different disorders, and diagnosis can be complex in the newborn period. The diagnosis of hypotonia in the NICU can be stepwise, and 50% of these can be caught by clinical examination, family history, and tests such as MRI or microarray tests for trisomy or MLPA tests. The conditions identified in this category can be hypoxic-ischemic encephalopathy (HIE), chromosomal disorders and/or Prader-Willi. The remaining 30-50% of hypotonia cases can be identified through additional testing such as amino acid disorder tests and biopsies. Some of these can have low rate of conclusive diagnosis. In one example, there are 131 hypotonia related genes in the NBDX v1.1 panel and if the incidence at a per gene level is calculated, then at least 2000 incidences in USA can be predicted or identified using the panel. This can mean that at least ˜2000 out of the remaining 3000-6000 cases can be identified by a single genetic test in one step instead of going through a 6 step clinical pathway to a final diagnosis. A total of 513 genes are currently considered in the in filter and can be made available as a second panel or part of an Exome test using the 513 genes as an in silico filter. This means the diagnostic rate can be at least 33-66%, assuming the symptoms presenting are 100% of the incidence, ho those cases where the diagnosis is negative, the standard algorithm or Exome analysis can be applied. This would significantly benefit the hypotonia patients who may have other suspected genetic conditions that currently are not testable.

In another example, hypoglycemia is a biochemical finding and understanding of the molecular mechanisms that lead to hypoglycemia can be important. At a genetic level, hypoglycemia can be due to many different genetic disorders including metabolic and endocrine conditions. Some of these genetic disorders present with severe and profound hypoglycemia in the newborn period yet others can be mild and subtle. Some of the metabolic and endocrine diseases are not screened for (e.g., congenital hyperinsulinism, defects in fructose metabolism, defects in glycogen synthesis and syndromes). Incidence of congenital hyperinsulinism is 1 in 35,000 or 40,000 or about 100 cases per year. Beckwith-Wiedemann syndrome is 1 in 14,000 or 300 cases per year. HFI is about 1 in 20,000 or 200 cases. Glycogen storage diseases are at 1 in 20,000 or 200 cases. Kabuki is about 3 per 100,000 or 120 per year. Chart review in a hospital in Boston suggest the incidence is 8% on patient intake of 7000, and 560 admissions in NICU. Those administered Diazoxide is about 5 per year. This suggests there are likely 28,000 hypoglycemia cases and ˜300 newborns on Diazoxide in USA. Thus 300-1000 cases out of 20,000 newborns could benefit from a molecular diagnosis.

### Conditions

The methods and systems disclosed herein can be used as differential analysis and/or confirmation of single gene conditions or a phenotype such as but not limited to, sickle cell disease, cystic fibrosis (CF), galactosemia, Maple syrup urine disease (MSUD), Glutaric acidemia type 1 (GA-1), Methylmalonic acidemia (MM), Psoriatic Arthritis (PA), 3-methyl-crotonylglycinuria. Phenylketonuria (PKU), and muscular dystrophy, as well as biotinidase, Glucose-6-phosphate dehydrogenase (G-6-PD), and Medium-chain acyl-CoA dehydrogenase (MCAD) deficiencies.

The methods and systems disclosed herein can be used as a second-tier molecular analysis, confirmation and/or differential diagnosis of genetic conditions. Second-tier testing can have the advantage of improving sensitivity and specificity of primary screening. It can also reduce parental anxiety and identify genotypes which can be associated with milder phenotypes, such as, but not limited to the Duarte variant in galactosemia, D444H in biotinidase deficiency, Δf508 in CF, and T199C in MCAD deficiency. For example, G-6-PD screening includes the common African-American double mutation (G202A; A376G) and the single (A376G) mutation; the Mediterranean mutation (C563T); and two Canton mutations (G1376T and G1388A).

The genetic condition disclosed in the methods and systems can comprise a disease, a phenotype, a disorder, or a pathogen. In some cases, determining the presence, absence or predisposition of a genetic condition comprises determining the predisposition or susceptibility to the genetic condition. In some cases, determining the presence, absence or predisposition of a genetic condition comprises determining the possibility of developing the genetic condition.

In some cases, the subject has a phenotype or is symptomatic. In some cases, the subject has a phenotype or is symptomatic of a disease. In some cases, the subject has no phenotype or is asymptomatic. In some cases, a Newborn Screening (NBS) has not been performed on the subject. In some cases, the subject has a phenotype or is symptomatic of for example, hypotonia, hepatosplenomegaly or failure to thrive. In some cases, the subject has no phenotype or is asymptomatic of a disease. In some cases, a Newborn Screening (NBS) or NBDx has been performed on the subject. In some cases, the subject has a result from one or more newborn screening tests such as tandem mass spectrometry results (metabolic disorders), Cystic Fibrosis (CF) screen, Severe combined immunodeficiency (SCID) (low TREC number), thyroid function, hemoglobin, and/or hearing. In some cases, the result from one or more newborn screening tests is not normal or inconclusive. In those cases, a further screening test based on the results from the newborn screening test can be performed, for example, a specific gene panel can be screened. In some cases, multiple screening tests can be performed on a subject. The screening tests as described herein can be based on one or more or combinations of exemplary gene panels described in Example 5.

In some cases, the subject is hospitalized. A neonate administered ototoxic drugs should know risk of exposure. In some subjects aminoglycosides (antibiotics) cause ototoxicity and induce hearing loss. Some subjects have mitochondrial mutations that make them predisposed to ototoxicity. In some cases, the disclosed method (e.g., genetic test) is performed prior to an antibiotic administration to the subject. In some cases, the disclosed method (e.g., genetic test) is performed after an antibiotic administration to the subject. In some cases, the disclosed method is performed while an antibiotic medication is administered to the subject.

In some cases a CMV-salivary PCR test has been performed on the subject. In some cases, an antiviral like ganciclovir is used to treat a CMV positive subjective. In some cases, a genetic test reveals cause of ganciclovir resistance when the subject (e.g. newborn) is unresponsive to the antiviral like ganciclovir.

### Samples

The methods and systems for detecting molecules (e.g., nucleic acids, proteins, etc.) in a subject who receives a screening test in order to detect, diagnose, monitor, predict, or screen the presence, absence or predisposition of a genetic condition are described in this disclosure. In some cases, the molecules are circulating molecules. In some cases, the molecules are expressed in blood cells. In some cases, the molecules are cell-free circulating nucleic acids.

The methods and systems disclosed herein can be used to screen one or more samples from one or more subjects. One or more samples can be obtained from a subject. One or more samples can be obtained from one or more subjects. In one example, one or more samples are obtained from a newborn subject. In another example, one or more samples are obtained from one or more relatives of the newborn subject. A sample can be any material containing tissues, cells, nucleic acids, genes, gene fragments, expression products, polypeptides, exosomes, gene expression products, or gene expression product fragments of a subject to be tested. Methods for determining sample suitability and/or adequacy are provided. A sample can include but is not limited to, tissue, cells, or biological material from cells or derived from cells of an individual. In some instances, the sample is a tissue sample or an organ sample, such as a biopsy. The sample can be a heterogeneous or homogeneous population of cells or tissues. In some cases, the sample is from a single patient. In some cases, the method comprises analyzing multiple samples at once, e.g., via massively parallel sequencing.

The sample can be a bodily fluid. The bodily fluid can be sweat, saliva, tears, wine, blood, menses, semen, and/or spinal fluid. In some aspects, the sample is a blood sample. The sample can be a whole blood sample. The blood sample can be a peripheral blood sample. In some cases, the sample comprises peripheral blood mononuclear cells (PBMCs). In some cases, the sample comprises peripheral blood lymphocytes (PBLs). The sample can be a serum sample. The blood sample can be fresh or taken previously. The blood sample can be a dried sample. The blood sample can be a dried blood spot.

The methods and systems disclosed herein can comprise specifically detecting, profiling, or quantitating molecules (e.g., nucleic acids, DNA, RNA, polypeptides, etc.) that are within the biological samples. In some instances, genomic expression products, including RNA, or polypeptides, can be isolated from the biological samples. In some cases, nucleic acids, DNA, RNA, polypeptides can be isolated from a cell-free source. In some cases, nucleic acids, DNA, RNA, polypeptides can be isolated from cells derived from the subject.

The sample can be obtained using any method known to the art that can provide a sample suitable for the analytical methods described herein. The sample can be obtained by a non-invasive method such as an oral swab, throat swab, buccal swab, bronchial lavage, urine collection, scraping of the skin or cervix, swabbing of the cheek, saliva collection, feces collection, menses collection, or semen collection. The sample can be obtained by a minimally-invasive method such as a blood draw. The sample can be obtained by venipuncture. The sample can be obtained by a needle prick. The sample can be obtained from the arm, the foot, the finger, or the heel of the subject. In other instances, the sample is obtained by an invasive procedure including but not limited to: biopsy, alveolar or pulmonary lavage, or needle aspiration. The method of biopsy can include surgical biopsy, incisional biopsy, excisional biopsy, punch biopsy, shave biopsy, and/or skin biopsy. The sample can be formalin fixed sections. The method of needle aspiration can further include fine needle aspiration, core needle biopsy, vacuum assisted biopsy, or large core biopsy, in some aspects, multiple samples can be obtained by the methods herein to ensure a sufficient amount of biological material. In some instances, the sample is not obtained by biopsy. Molecular autopsy can be another application for sudden infant deaths or cardiac cases. In some aspects, molecular autopsy samples could be different due to fixative like formaldehyde for fixing cells, tissues etc.

Blood and other body fluids contain both cells and cell-free form (e.g. plasma). In some cases, the cell-free DNA isolation methods can be used in prenatal testing environment as fetal DNA traverses barrier to enter maternal circulation. In some cases, the cell-free DNA isolation methods can be used in a post-natal setting like newborn's blood to separate or enrich blood-borne pathogens and/or nucleic acids. In some cases, the cell-free DNA isolation methods can be used in a newborn blood to look at causes of sepsis and by removing contaminating human DNA that are in cell free form and/or within white blood cells (WBCs).

The isolation methods can involve rupturing WBCs to release the high molecular weight human DNA. In body fluids, the human DNA fraction can be in vast excess given its size of 3×10⁹ bp and high number of cells. Some portion of the human DNA can also exist in body fluids as either fragmented form in cell-free fraction (nucleosomal bound or fragmented). In contrast, a bacterial genome can be much smaller—200×10³ bp. In blood, even in cases of sepsis, the number of bacterial cells over human nucleated WBCs can be 10³ fold less. Thus the proportion of cells and genome size can make detection and analysis of pathogens a challenge. In contrast, the methods and systems disclosed herein can detect and analyze pathogens by removing WBCs and the genomes in WBC. In some cases, pathogen nucleic acids can be in the body fluids. In some cases, pathogen nucleic acids can be in naked form. In some cases, pathogen nucleic acids can be inside or outside a cellular structure. For example, pathogen nucleic acids can be in a bacterial cell. In some cases, pathogen nucleic acids can be a bacterial DNA that is enriched and/or measured in saliva. In some cases, pathogen nucleic acids can be a large undegraded viral DNA like human cytomegalovirus.

The methods and systems disclosed herein can be used in isolation of pathogen DNA from endogenous DNA. In one aspect, DNA from cell fraction of human body fluids can be isolated. In another aspect, DNA from cell-free fractions of human body fluids can be isolated. The isolation of DNA from cell and/or cell-free fractions of human body fluids can be accomplished by simple centrifugation of whole blood or body fluid. The isolation of DNA fraction from cell and cell-free fractions of human body fluids can be accomplished by centrifugation in presence of ficoll-gradient. The isolation of DNA can be accomplished by removal of cellular DNA. In some cases, the isolated DNA can be a small amount of endogenous DNA. In some cases, the isolated DNA can be pathogen DNA. Alternatively, pathogen RNA can also be isolated. In essence, this is a subtraction of the endogenous genome and enrichment of the pathogen genome. Isolation of pathogen DNA from endogenous DNA can also be used in massive parallel sequencing.

Endogenous cell-free DNA can be fragmented. The endogenous cell-free DNA can be less than 1000 bp in size, for example, less than 10, 20, 30, 40, 50, 60, 70, 80, 90, 100, 150, 200, 250, 300, 350, 400, 450, 500, 550, 600, 650, 700, 750, 800, 850, 900, 950, or 1000 bp in size. The isolation of DNA can be accomplished by removal of a particular size of endogenous cell-free DNA and enrich for pathogen DNA in a different size fraction. The pathogenic DNA can be more than 1000 bp in size, for example, more than 10, 20, 30, 40, 50, 60, 70, 80, 90, 100, 150, 200, 250, 300, 350, 400, 450, 500, 550, 600, 650, 700, 750, 800, 850, 900, 950, or 1000 bp in size.

In one aspect, single stranded nucleic acids can be distinguished and/or isolated from double stranded nucleic acids. In some cases, it is achieved by enzymatic digestion methods. In some cases, the enzymatic digestion method generates a double-stranded DNA break. In some cases, it is achieved by recognizing a species specific DNA signature. In some cases, the species specific DNA signature is a methylation site (e.g. CpG or CHG sites). In some cases, the species specific DNA signature is a ^{m}CNNR (R can be A or G; N can be A, C, G, or T; ^{m}C can be cytosine modifications include C5-methylation (5-mC) and C5-hydroxymethylation (5-hmC)) site. In some cases, the species specific DNA signature is recognized by MspJI.

### Sample Collection

DNA isolation for NGS can involve collecting several milliliters (e.g. 2-10 mL) of whole blood from the patient. For newborns, that level of sample collection can pose a danger in itself, especially for premature and/or otherwise sick babies, or delays due to secondary blood draws. Alternative minimally invasive methods such as use of dried blood spots (DBS), single blood drops, cord blood, small volume whole blood and/or saliva can be used for newborn tests with fast turnaround times.

The disclosed methods and systems can use a whole blood sample. In some cases, the methods and systems further comprises purifying a DNA from the sample. In some cases, the methods and systems does not comprise purifying a DNA from the sample.

The disclosed methods and systems can use a low-volume of a sample (e.g. DBS). In some cases, the method uses 1-500 μL of the sample. For example, 1-500, 1-300, 1-100, 1-80, 1-60, 1-40, 1-20, 1-10, 1-5, 10-500, 10-300, 10-100, 10-80, 10-60, 10-40, 10-20, 20-500, 20-300, 20-100, 20-80, 20-60, 20-40, 40-500, 40-300, 40-100, 40-80, 40-60, 60-500, 60-300, 60-100, 60-80, 80-500, 80-300, 80-100, 100-500, 100-300, or 300-500 μL of the sample (e.g. DBS) can be used. In some cases, the method uses less than 1000 μL of the sample. For example, less than 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950 or 1000 μL of the sample (e.g. DBS) can be used. In some cases, the method uses less than 10 spots of the DBS sample. For example, the method uses less than 10, 9, 8, 7, 6, 5, 4, 3, 2, 1, ½, ⅓, ¼, ⅕, ⅙, 1/7, ⅛, 1/9, or 1/10 spot(s) of the DBS sample. The remaining sample can be preserved for future use. In some cases, the sample is used after a period of time, such as 1 hour, 2 hours, 3 hours, 4 hours, 5 hours, 6 hours, 12 hours, 24 hours, 36 hours, 48 hours, 3 days, 4, days, 5 days, 6 days, 7 days, 2 weeks, 3 weeks, 4 weeks, 1 month, 2 months, 3 months, 4 months, 5 months, 6 months, 1 year, 2 years, 3 years, 4 years, or 5 years after its collection. The disclosed methods and systems can also use a low-volume DNA isolation method from a sample (e.g. DBS).

Alternatively, saliva and/or buccal smear can be used for sample collection. A rayon swab can be used to collect saliva. The sample can be kept in a solution to protect from DNA degradation and microbial growth. These sample collection methods can provide good alternatives to families with aversion to invasive blood collection. Additionally, DBS samples (e.g. Guthrie Cards) do not have added preservatives and DNA obtained from whole blood or DBS can be degraded. Such samples can still be utilized in the TNGS workflow as described herein.

Various collection devices can be used to improve DNA recovery from a sample. In some cases, blood spots can be dried to materials that more readily release DNA. For example, the collection and transportation device can comprise a material in the form of a card or blotter. The material can be hydrophilic and/or negatively charged. The material can comprise a cellulose, rayon or nylon.

In one aspect, a device to collect saliva can be used. In some cases, the device has a plastic lid and a container. The container can hold a filter paper of a defined size and a swab can be placed in contact with the filter paper. The device improves the trapping in a defined space, captures additional volume and/or captures a fixed volume irrespective of viscosity variations in body fluids.

### DNA Recovery Techniques

DNA can be recovered from a sample, for example, DBS on cloth, cotton swabs and/or cellulose fibers. The methods and systems disclosed herein can use different lysis buffer compositions, pressure levels, number of pressure cycles, total durations of pressure cycling and temperatures. In some cases, the methods and systems disclosed herein uses a variety of buffer additives to aide cell lysis (e.g. non-ionic detergent) or mitigate PCR inhibition including BSA, DMSO, betaine and/or chelex resin. In some cases, the methods and systems disclosed herein uses a lysis buffer pH of 1-14, for example, a lysis buffer pH of about 1, 2, 3, 4, 5, 6, 9, 10, 11, 12, 13, or 14. In some cases, the methods and systems disclosed herein uses a pressure cycling at 1000 to 100000 psi, for example, a pressure cycling at 1000, 2000, 3000, 4000, 5000, 6000, 7000, 8000, 9000, 10000, 15000, 20000, 25000, 30000, 35000, 40000, 45000, 50000, 55000, 60000, 65000, 70000, 75000, 80000, 85000, 90000, 95000, or 100000 psi. In some cases, the methods and systems disclosed herein uses 1-500 pressure cycles, for example, 1, 2, 3, 4, 5, 6, 7, 8, 9, 10, 20, 30, 40, 50, 60, 70, 80, 90, 100, 200, 300, 400, or 500 pressure cycles. In some cases, the methods and systems disclosed herein include a 1 min to 10 hours of total durations of pressure cycling. For example, the total durations of pressure cycling is 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, or 10 hours. In some cases, the methods and systems disclosed herein is performed at 10° C. to 100° C., for example, at 10, 20, 30, 40, 50, 60, 70, 80, 90, or 100° C. some cases, the methods and systems disclosed herein use a pressure cycling including a process at high pressure followed by another process at atmospheric pressure (14.7 psi). In some cases, Proteinase K is used in the DNA recovery methods.

The disclosed methods and systems provide sufficient yield and quality of double-stranded DNA from DBS. The GENSOLVE™ Reagent from IntegenX for cell lysis and silica-based columns from the QIAAMP™ Mini Blood kit can be used for DNA isolation. The disclosed methods and systems can be used to isolate more than 10 μg DNA per spot from a DBS sample. For example, the disclosed methods and systems are used to isolate more than 1, 2, 3, 4, 5, 6, 7, 8, 9, 10, 20, 30, 40, 50, 60, 70, 80, 90, 100, 200, 300, 400, 500, 600, 700, 800, 900, or 1000 ng DNA per spot from a DBS sample. In some cases, the disclosed methods and systems are used to isolate more than 1, 2, 3, 4, 5, 6, 7, 9, or 10 μg DNA per spot from a DBS sample.

DNA yields, intact quality, lack of contamination and purity from inhibitors can be measured and/or monitored by double-strand specific assay, for example, the QUBIT™ assay. This assay has advantages over other OD260 spectrophotometric assays (e.g. NANODROP™) in two aspects: 1) Lower limit of detection for accurate measurement of limited DNA material. 2) Better specificity to the double-stranded DNA (dsDNA), separate from single-stranded DNA (ssDNA) and other contaminants that influence OD260, generally used for ligation or tagmentation driven NGS library production. The intact quality of genomic DNA can be measured by agarose gel electrophoresis, DNA from the gold standard methods has been demonstrated at >50 kb, NGS can have ever increasing sequencing read lengths and for specific assays used for completing genomic analysis such as long range amplification for pseudogenes, mapping haplotypes and cis/trans phasing of heterozygous variants, genomic rearrangements and matepair library production. Finally, isolated DNA can be tested for purity from enzymatic inhibitors using a highly sensitive quantitative PCR assay for an Internal Positive Control (IPC) of non-human DNA spiked into the PCR reaction. This is an established assay and can be used to assess isolated gDNA. Samples containing even low levels of inhibitors cause the IPC to amplify at later cycles.

DNA can be recovered from a sample, for example, liquid blood. Differential lysis of white blood cells (WBC) and red blood cells (RBC) can be used the methods and systems disclosed herein.

The methods and systems disclosed herein can recover DNA without denaturing DNA. In some cases, the recovered DNA is in double stranded format. In other cases, the recovered DNA is in single stranded format. In some cases, the recovered DNA has more single stranded DNA than double stranded DNA. However, the recovered DNA can have more double stranded DNA than single stranded DNA. In some cases, more than 50% of the recovered DNA is double stranded DNA. For example, more than 95%, 90%, 85%, 80%, 75%, 70%, 65%, 60%, 55%, or 50% of the recovered DNA is double stranded DNA.

Double stranded DNA can be used for subsequent application in next generation sequencing workflow because in many such applications a synthetic adapter is ligated for sample barcoding, strand barcoding and DNA amplification into the double stranded DNA. Double stranded DNA can also be used for transposition based barcode, adapter integration, cellular heterogeneity, verification of true variation, and/or cis-trans confirmations.

Various technical improvements can be used for DNA recovery from a sample. In some cases, titration of number of dried blood spot (DBS) punches is used for DNA recovery, e.g. optimize lysis and DNA recovery. In some cases, high speed vortex incubations are used for DNA recovery, e.g. to assist in hydration of DBS and lysis. For example, vortex speed of at least about 0.10, 0.20, 0.30, 0.40, 0.50, 0.60, 0.70, 0.80, 0.90, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 12.0, 14.0, 160, 180, 20.0, 30.0, 40.0, 50.0, 60.0, 70.0, 80.0, 90.0 or 100.0 krpm is used for DNA recovery. In some cases, increased lysis solution volume is used for DNA recovery, e.g. to improve lysis. For example, lysis solution volume of at least about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 20, 30, 40, 50, 60, 70, 80, 90, 100, 200, 300, 400, 500, 600, 700, 800, 900 or 1000 mL is used for DNA recovery. In some cases, spin basket is used for DNA recovery, e.g. for separating DBS paper from blood after sample lysis. In some cases, titration of ethanol addition is used prior to column purification for DNA recovery. In some cases, wash buffer incubations on column is used for DNA recovery, e.g. to clean sample DNA. In some cases, additional washes, e.g., for an archival sample, is used for DNA recovery, e.g. to ensure removal of nuclease contaminants. For example, at least 1, 2, 3, 4, 5, 6, 7, 8, 9, or 10 washes are used for DNA recovery. In some cases, multiple-step elution of DNA from columns is used for DNA recovery. For example, at least 2, 3, 4, 5, 6, 7, 8, 9, or 10-step elution of DNA from columns is used for DNA recovery. In some cases, titration of EDTA concentration in a DNA elution is used for DNA recovery. In some cases, titration of EDTA concentration in a DNA elution is used to prevent nuclease action and/or allow downstream enzymatic reactions. In some cases, treatment of DBS with sodium bicarbonate is used for DNA recovery, e.g. for better DNA release. In some cases, treatment of DBS punches with Covaris is used for DNA recovery, e.g. to reduces the DNA fragment size.

Whole Genome Amplification (WGA) can be used in the methods and systems disclosed herein. In some cases, WGA is a method for robust amplification of an entire genome, starting with small quantities of DNA and resulting in much larger quantities of amplified products. Several methods ca used for high-fidelity whole genome amplification, including Multiple Displacement Amplification (MDA), Degenerate Oligonucleotide PCR (DOP-PCR) and Primer Extension Preamplification (PEP).

### Systems

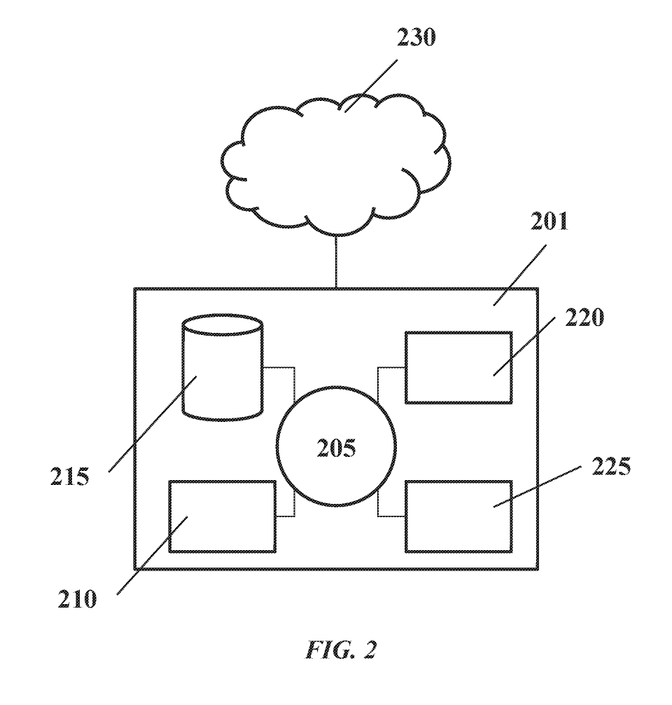

The present disclosure provides computer or digital systems that are programmed to implement methods of the disclosure. FIG. 2 shows a computer that is programmed or otherwise configured to implement methods of the disclosure. The computer system 201 can regulate various aspects of genotype analysis of the present disclosure, such as, for example, analysis by inheritance pattern scores, and/or analysis by association pattern scores.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which can enable devices coupled to the computer system 201 to behave as a client or a server.

The CPU 205 can execute a sequence of machine-readable instructions, which can be embodied in a program or software. The instructions can be stored in a memory location, such as the memory 210. Examples of operations performed by the CPU 205 can include fetch, decode, execute, and writeback.

The storage unit 215 can store files, such as drivers, libraries and saved programs. The storage unit 215 can store programs generated by users and recorded sessions, as well as output(s) associated with the programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology can be thought of as “products” or “articles of manufacture” e.g., in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer, or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instructions to a processor for execution.

The computer system 201 can include or be in communication with an electronic display that comprises a user interface (UI) for providing, for example, a display, graph, chart and/or list in graphical and/or numerical form of the genotype analysis according to the methods of the disclosure, which can include inheritance analysis, causative variant discovery analysis, and diagnosis. Examples of UI's include, without limitation, a graphical user interface (GUI) and web-based user interface.

The data generated by the ranking can be displayed (e.g., on a computer). The data can be displayed in a numerical and/or graphical form. For example, data can be displayed as a list, as statistics (e.g., p-values, standard deviations), as a chart (e.g., pie chart), as a graph (e.g., line graph, bar graph), as a histogram, as a map, as a heat map, as a timeline, as a tree chart, as a flowchart, as a cartogram, as a bubble chart, a polar area diagram, as a diagram, as a stream graph, as a Gantt chart, as a Nolan chart, as a smith chart, as a chevron plot, as a plot, as a box plot, as a dot plot, as a probability plot, as a scatter plot, and as a biplot, or any combination thereof.

Pseudogenes and/or High Homology Regions

The methods and systems disclosed herein integrate a solution for pseudogenes and/or high homology regions such as CYP21A2. These pseudogenes and/or high homology regions can interfere with TNGS mutation detection due to pseudogene mismapping after successful capture. In some cases, the methods and systems identify pseudogenes and/or high homology regions, such as CYP21A2. In some cases, the methods and systems cover pseudogenes and/or high homology regions, such as CYP21A2. In some cases, the methods and systems is able to confirm congenital adrenal hyperplasia.

The methods and systems disclosed herein can be used to identify pseudogenes and/or high homology regions, e.g. regions of homology between the 126 genes from NBDx and the whole genome. In some cases, computational pipelines, such as adapted from PSEUDOPIPE™, can be used. In some cases, evaluation of introns can be used for designing probes and amplicon primers. A two-step process can be used to identify target gene homology: 1) Search homology of the target gene sequences in the human genome by using BLAT64, followed by filtering of the alignment results. Gaps that are longer than the target genes can be removed in a BLAT alignment. In addition, a BLAT alignment whose total matching sequence length is shorter than the sequenced read length can be removed. For the whole genome the GRCh37 reference genome plus a decoy genome that contains about 36 MB of human genome sequence absent in the reference genome, such as processed pseudogenes and high homology, can be used in the methods and systems. 2) Pairwise alignment between the processed BLAT results and the target genes using global and local pairwise alignment tools such as Needle (using the Needleman-Wunsch algorithm) and/or Water (using the Smith-Waterman algorithm). Homology matches from pairwise alignments can be assessed using a sliding window analysis. The length of sliding window can correspond to the read length, e.g. 5, 10, 15, 20, 25, 30, 35, 40, 45, 50, 55, 60, 65, 70, 75, 80, 85, 90, 95 or 100 bp. For every window, it can be tested whether the pair of sequences matches perfectly allowing up to 1, 2, 3, 4, 5, 6, 7, 8, 9, or 10 base-pair mismatches. For example, it can be tested whether the pair of sequences matches perfectly allowing up to 2 base-pair mismatches.

The Burrows-Wheeler Aligner (BWA) can be used to map sequence reads to a reference (e.g. GRCh37) plus decoy genome, allowing reads to be mapped to multiple positions. The methods and systems can identify genomic loci from where the reads originated and/or identify potentially mismapped reads. Read pairs where one read is mapped uniquely but the other is mapped to homologous regions can be identified using the methods and systems, especially for paired-end or mate-pair sequencing. Paired read distance and/or realignment, can be used to confirm whether the reads mapped to homologous regions are derived from the target genes. Paired read distance and/or realignment, can be used to call variants. Correct mapping of reads can reduce false positive/low quality variant calls.

### Calling Variants in Regions of High Homology

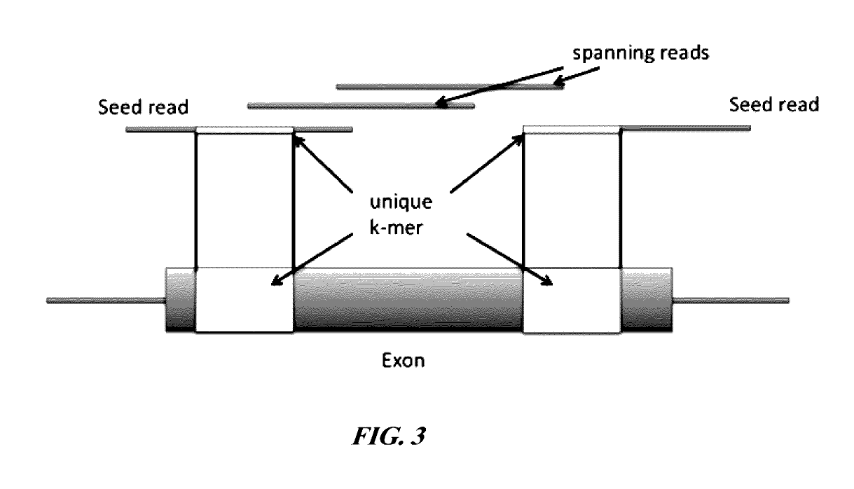

The methods and systems disclosed herein (e.g., including hybrid capture) can resolve calls in regions of high homology by searching unique k-mers (k={12, 24, 36, 72}) in the reference genome at loci of interest. As shown in FIG. 3, the sequence reads that match the same unique k-mer can be identified and used as “seed” reads. Contiguous sequence (e.g., a contig) can be then built between the seed reads so as to span a highly homologous genomic region. Contigs can be aligned back to the reference genome and variants can be called off the alignment. While fragment lengths used in standard hybrid capture libraries can be smaller (200-300 bp), larger fragments 1 Kb and above can also be used in capture followed by mate-pair strategies that circularizes long captured fragments, followed by fragmentation and sequencing or direct sequencing using Minion (when available) or PacBio RS II sequencers.

Amplicon analysis can be used in the methods and systems disclosed herein. Through this approach, only the correct gene is amplified, giving a high enrichment rate of the target in comparison to potential mis-mapping regions. When coupled with the bioinformatic analysis of panel content, design of priming regions and post-sequencing read mapping, the resulting sequencing reads can be mapped correctly. In some cases, generation of singleplex amplicons ready for NGS on the MiSeq sequencer (Illumina; lengths ˜300-700 bp) can be used. In some cases, long-range PCR of up to 10-kb amplicons for genes in which unique priming regions are not optimal. Wafergen can be used for production of Illumina-ready amplicons. Long-Range PCR can be performed on the Wafergen chips and also by direct sequencing on a PacBio RS II sequencer. In addition, matepair strategies that circularize long amplicons followed by fragmentation for sequencing on MiSeq can also be used. Amplicon assays can utilize the nCounter instrument (Nanostring).

The methods and systems disclosed herein can be used for identifying and/or validating Cystic Fibrosis (CF) and/or Cystic fibrosis transmembrane conductance regulator (CFTR) related metabolic syndrome (CRMS). Validations can be performed by identification of CFTR variants by TNGS with an silico screen for the CA40 mutation panel. Validations can be performed by identification of the remaining TNGS intronic and exonic CFTR sequence, e.g. to emulate the two DNA interrogation steps in the current CA CF NBS algorithm. For validation of carriers (e.g. elevated IRT and one detected CA40 mutation) without a second variant present, a full CFTR TNGS can be performed.

### Pathogen Detection

The methods and systems disclosed herein can be used to identify a pathogen in a sample. Pathogens can include Polio virus, Human papilloma virus, Bacillus anthracis, Bacillus cereus, Clostridium botulinum, Brucella spp., Campylobacter spp., Carbapenem-resistant Enterobacteriaceae, Haemophilus ducreyi, Varicella-zoster virus, Chikungunya virus, Chlamydia trachomatis, Vibrio cholerae, Clostridium difficile, Clostridium perfringens, Cryptosporidium panium, hominis, Cytomegalovirus (CMV), Dengue virus, Corynebacterium diphtheriae or ulcerans, Echinococcus spp., Enterococcus spp., Escherichia coli, Giardia lamblia, Neisseria gonorrhoeae, Klebsiella granulomatis, Haemophilus influenzae, Hepatitis A virus, Hepatitis B virus, Hepatitis C virus, Herpes simplex virus, Human immunodeficiency virus, Influenza A and B virus, Klebsiella pneumoniae, Legionella spp., Mycobacterium leprae, Leptospira spp., Listeria monocytogenes, Borrelia burgdorferi, Chlamydia trachotnatis, Plasmodiwn falciparum, vivax, knowlesi, ovate, malariae, Measles virus, Neisseria meningitidis, Mumps virus, Norovirus, Salmonella Paratyphi, Bordetella pertussis, Yersinia pestis, Pseudomonas aeruginosa, Coxiella bumetii, Rabies virus, Respiratory syncytial virus, Rotavirus, Rubella virus, Salmonella spp. other than S. Typhi and S. Paratyphi, Severe Acute Respiratory Syndrome (SARS)-associated coronavirus, Shigella spp., Variola virus, Enterotoxigenic Staphylococcus aureus, Staphylococcus aureus, Streptococcus pyogenes, Streptococcus agalactiae, Streptococcus pneumoniae, Treponema pallidum, Clostridium tetani, Toxoplasma gondii, Trichinella spp., Trichomonas vaginalis, Mycobacterium tuberculosis complex, Francisella tularensis, Salmonella Typhi, Rickettsia prowazekii, Verotoxin producing Escherichia coli, West Nile virus, Yellow fever virus, Yersinia enterocolitica, and Yersinia pseudotuberculosis.

Pathogens can also include Sepsis, Rubella, Botulism, Gram-negative bacteria such as Klebsiella (pneumoniae/oxytoca), Serratia marcescens, Enterobacter (cloacae/aerogenes), Proteus mirabilis, Acinetobacter baumannii, and Stenotrophomonas maltophilia; Gram-positive bacteria such as CoNS (Coagullase negative Staphylococci), Enterococcus faecium, Enterococcus faecalis; and Fungi such as Candida albicans, Candida tropicalis, Candida parapsilosis, Candida krusei, Candida glabrata, Aspergillus fumigatus.

The methods and systems disclosed herein such as hybrid selection can be used to isolate specific pathogen DNA using a library of probes to identify a pathogen in a sample. An alternative can be the titration and/or depletion of human sequences by the methods and systems. The titration can span a range that mimics infection positive clinical samples, including a non-infection control, with starting DNA matching typical yields from cord blood or venipuncture of newborns. Each titration point can be split for a pre-isolation control and testing of subtractive methodologies for depletion of human sequences. The analysis can be done by comparing the results with an infection positive clinical sample and a non-infection control. The comparison can include relative yield of pathogen to human sequences, minimal pathogen detection level, time to results and accuracy of detection.

The methods and systems disclosed herein can be used to identify microbiome and/or pathogenic organisms. The methods and systems disclosed herein can be used to populate a database for microbiome and/or pathogenic organisms. The methods and systems disclosed herein can be used to identify previously unknown organisms.

Human DNA can be depleted to allow focused NGS on microbiome and/or pathogenic organisms. Titration points can be prepared into sequencing libraries and split four ways to give anon-subtracted control and/or three subtraction method tests. The methods and systems disclosed herein can comprise depleting human genome signal from the sequencing product. In some cases, the depleting human genome signal comprises in silico subtraction of the human genome signal. The methods and systems can result in at least about 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 30, 40, 50, 60, 70, 80, 90, 100, 200, 300, 400, 500, 600, 700, 800, 900, or 1000-fold increase in number of reads of the pathogen genome as compared to an untreated control. In some cases, the methods and systems result in at least about 1, 2, 4, 5, 6, 7, 8, 9, 10, 20, 30, 40, 50, 60, 70, 80, 90, 100, 200, 300, 400, 500, 600, 700, 800, 900, or 1000-thousand fold increase in number of reads of the pathogen genome as compared to an untreated control. In some cases, the methods and systems result in at least about 1, 2, 3, 4, 5, 6, 7, 8, 9, or 10-million fold increase in number of reads of the pathogen genome as compared to an untreated control.

MspJI (a nuclease that cuts at fully methylated CpG and CBG sites) digestion can be used to enrich microbiome and/or pathogenic organisms. For example, cleavage by MspJI or any other suitable enzyme can be used to enrich malaria in clinical samples and result in an about 9-fold increase in number of reads of the pathogen e.g. malarial) genome as compared to the untreated control.

Depletion of highly repetitive sequences can be used to deplete human genome via duplex-specific nuclease (DSN) treatment of samples, e.g. partially renatured samples. The human genome is about 50% repetitive elements, as compared to about 1.5% in bacterial genomes. DSN specifically can cleave DNA duplexes while retaining ssDNA. Prior to DSN, samples can be fully heat denatured and partially renatured to allow highly repetitive sequences to hybridize as per cot association kinetics.

Alternately, hybridization can be used to deplete human genome. A low-cost Whole Genome Bait (WGB) library can be produced from uninfected human DNA through fragmentation, ligation to a T7 promoter and in vitro transcription. This method can cause a high degree of human-specific sequence depletion, without requiring more work to establish the WGB library and a workflow for effective hybridization.

The enzymatic approaches can reduce process time and/or increase pathogen identification level. Synthesized probe libraries can used to recover pathogen sequences for NGS. The methods and systems, e.g. MspJI digestion and/or DSN treatment, can have a turnaround time of less than 120 hours. For example, the methods and systems can have a turnaround time of less than 120, 108, 96, 84, 72, 60, 48, 36, 24, 12, 6, 5, 4, 3, 2 or 1 hour. The methods and systems can further comprise a streamlined NGS library method (e.g. Nextera, Fragmentase). In some cases, the methods and systems cannot be limited to use known pathogen sequences.

Evaluation of the reduction of human DNA can be performed by comparison of pre- and post-depletion samples via RT-PCR. Reduction of human DNA can be tracked via primer pairs for high copy human sequences (e.g. Actin, GADPH). Reduction of human DNA can be tracked via enrichment of non-endogenous sequences. For example, reduction of human DNA can be tested through the commonly tested bacterial high copy 16S rRNA gene and/or single copy uidA. Sequencing analysis can be done on the MiSeq (Illumina).

The methods and systems can be used for pathogen detection and identification. The pathogen can be a known organism. The pathogen can be an unknown organism. The methods and systems can compare the sequencing result with a database in the Microbiome Project. The methods and systems can use PathSeq, which utilizes a multi-stage alignment and filtering approach to partition human and microbial reads. Microbial reads can be aligned against known sequences and de novo assembled for possible identification of previously unknown organisms. The methods and systems can be used for determining the microbial resistance type.

### Sequencing

In some instances, data to be analyzed by the methods of the disclosure can comprise sequencing data. Sequencing data can be obtained by a variety of techniques and/or sequencing platforms. Sequencing techniques and/or platforms broadly fall into at least two assay categories (for example, polymerase and/or ligase based) and/or at least two detection categories (for example, asynchronous single molecule and/or synchronous multi-molecule readouts).

In some instances massively parallel high throughput sequencing techniques can avoid molecular cloning in a microbial host (for example, transformed bacteria, such as E. coli) to propagate the DNA inserts. Massively parallel high throughput sequencing techniques can use in vitro clonal PCR amplification strategies to meet the molecular detection sensitivities of the current molecule sequencing technologies, Some sequencing platforms (e.g., Helicos Biosciences) can avoid amplification altogether and sequence single, unamplified DNA molecules. With or without clonal amplification, the available yield of unique sequencing templates can have a significant impact on the total efficiency of the sequencing process.

Sequencing can be performed by sequencing-by-synthesis (SBS) technologies. SBS can refer to methods for determining the identity of one or more nucleotides in a polynucleotide or in a population of polynucleotides, wherein the methods comprise the stepwise synthesis of a single strand of polynucleotide complementary to the template polynucleotide whose nucleotide sequence is to be determined. An oligonucleotide primer can be designed to anneal to a predetermined, complementary position of the sample template molecule. The primer/template complex can be presented with a nucleotide in the presence of a nucleic acid polymerase enzyme. If the nucleotide is complementary to the position on the sample template molecule that is directly adjacent to the 3′ end of the oligonucleotide primer, then the polymerase can extend the primer with the nucleotide. Alternatively, the primer/template complex can be presented with all nucleotides of interest (e.g., adenine (A), guanine (G), cytosine (C), and thymine (T)) at once, and the nucleotide that is complementary to the position on the sample template molecule directly adjacent to the 3′ end of the oligonucleotide primer can be incorporated. In either scenario, the nucleotides can be chemically blocked (such as at the 3″-0 position) to prevent further extension, and can be deblocked prior to the next round of synthesis, Incorporation of the nucleotide can be detected by detecting the release of pyrophosphate (PPi), via chemiluminescence, or by use of detectable labels bound to the nucleotides. Detectable labels can include mass tags and fluorescent or chemiluminescent labels. The detectable label can be bound directly or indirectly to the nucleotides. In the case of fluorescent labels, the label can be excited directly by an external light stimulus, or indirectly by emission from a fluorescent (FRET) or luminescent (LRET) donor. After detection of the detectable label, the label can be inactivated, or separated from the reaction, so that it cannot interfere or mix with the signal from a subsequent label. Label separation can be achieved, for example, by chemical cleavage or photocleavage. Label inactivation can be achieved, for example, by photobleaching.

Sequencing data can be generated by sequencing by a nanopore-based method. In nanopore sequencing, a single-stranded DNA or RNA molecule can be electrophoretically driven through a nano-scale pore in such a way that the molecule traverses the pore in a strict linear fashion. Because a translocating molecule can partially obstruct or blocks the nanopore, it can alter the pore's electrical properties. This change in electrical properties can be dependent upon the nucleotide sequence, and can be measured. The nanopore can comprise a protein molecule, or it can be solid-state. Very long read lengths can be achieved, e.g. thousands, tens of thousands or hundreds of thousands of consecutive nucleotides can be read from a single molecule, using nanopore-based sequencing.

Another method of sequencing suitable for use in the methods of the disclosure is pyrophosphate-based sequencing. In pyrophosphate-based sequencing, sample DNA can be sequenced and the extension primer subjected to a polymerase reaction in the presence of a nucleotide triphosphate whereby the nucleotide triphosphate can become incorporated and release pyrophosphate (PPi) if it is complementary to the base in the target position, the nucleotide triphosphate being added either to separate aliquots of sample-primer mixture or successively to the same sample-primer mixture. The release of PPi can be detected to indicate which nucleotide is incorporated. In some aspects, a region of the sequence product can be determined by annealing a sequencing primer to a region of the template nucleic acid, and contacting the sequencing primer with a DNA polymerase and a known nucleotide triphosphate, (i.e., dATP, dCTP, dGTP, dTTP), or an analog of one of these nucleotides. The sequence can be determined by detecting a sequence reaction byproduct. The sequence primer can be any length or base composition, as long as it is capable of specifically annealing to a region of the amplified nucleic acid template. No particular structure for the sequencing primer is required so long as it can specifically prime a region on the amplified template nucleic acid. The sequencing primer can be complementary to a region of the template that is between the sequence to be characterized and the sequence hybridizable to the anchor primer. The sequencing primer can be extended with the DNA polymerase to form a sequence product. The extension can be performed in the presence of one or more types of nucleotide triphosphates, and if desired, auxiliary binding proteins.

Incorporation of the dNTP can be determined by assaying for the presence of a sequencing byproduct. The nucleotide sequence of the sequencing product can be determined by measuring inorganic pyrophosphate (PPi) liberated from a nucleotide triphosphate (dNTP) as the dNMP is incorporated into an extended sequence primer. This method of sequencing can be performed in solution (liquid phase) or as a solid phase technique.

Sequencing can be performed by SOLiD sequencing. The SOLiD platform can use an adapter-ligated fragment library similar to those of the other next-generation platforms, and can use an emulsion PCR approach with small magnetic beads to amplify the fragments for sequencing, Unlike the other platforms, SOLiD can use DNA ligase and a unique approach to sequence the amplified fragments. Two flow cells can be processed per instrument run, each of which can be divided to comprise different libraries in up to four quadrants. Read lengths for SOLiD can be user defined between 25-50 bp, and each sequencing run can yield up to −100 Gb of DNA sequence data, Once the reads are base called, have quality values, and low-quality sequences have been removed, the reads can be aligned to a reference genome to enable a second tier of quality evaluation called two-base encoding.

Sequencing can be performed by polony sequencing methods. A polony (or PCR colony) can refer to a colony of DNA that is amplified from a single nucleic acid molecule within an acrylamide gel such that diffusion of amplicons is spatially restricted. A library of DNA molecules can be diluted into a mixture that comprises PCR reagents and acrylamide monomer. A thin acryl amide gel (approximately 30 microns (μm)) can be poured on a microscope slide, and amplification can be performed using standard PCR cycling conditions. A library of nucleic acids such that a variable region is flanked by constant regions common to all molecules in the library can be used such that a single set of primers complementary to the constant regions can be used to universally amplify a diverse library. Amplification of a dilute mixture of single template molecules can lead to the formation of distinct, spherical polonies. Thus, all molecules within a given polony can be amplicons of the same single molecule, but molecules in two distinct polonies can be amplicons of different single molecules. Over a million distinguishable polonies, each arising from a distinct single molecule, can be farmed and visualized on a single microscope slide.

An amplification primer can include a 5′-acrydite-modification. This primer can be present when the acrylamide gel is first cast, such that it physically participates in polymerization and is covalently linked to the gel matrix. Consequently, after PCR, the same strand of every double-stranded amplicon can be physically linked to the gel. Exposing the gel to denaturing conditions can permit efficient removal of the unattached strand. Copies of the remaining strand can be physically attached to the gel matrix, such that a variety of biochemical reactions on the full set of amplified polonies in a highly parallel reaction can be performed. A polony can refer to a DNA-coated bead rather than in situ amplified DNA and 26-30 bases can be sequence from 1.6×10⁹ beads simultaneously. It can be possible to scale-up the sequencing to 36 continuous bases (and up to 90 bases) from 2.8×10⁹ beads simultaneously and can be as many at 10¹⁰.

### Untargeted Sequencing

Untarget-specific sequencing cal be used as a method for generating sequencing data. The methods can provide sequence information regarding one or more polymorphisms, sets of genes, sets of regulatory elements, micro-deletions, homopolymers, simple tandem repeats, regions of high GC content, regions of low GC content, paralogous regions, or a combination thereof. In some cases, the untargeted sequencing can be whole genome sequencing. In some cases, the untargeted sequencing data can be the untargeted portion of the data generated from a target-specific sequencing assay. The methods can generate an output comprising a combined data set comprising target-specific sequencing data and a low coverage untargeted sequencing data as supplement to target-specific sequencing data. Non-limiting examples of the low coverage untargeted sequencing data include low coverage whole genome sequencing data or the untargeted portion of the target-specific sequencing data. This low coverage genome data can be analyzed to assess copy number variation or other types of polymorphism of the sequence in the sample. The low coverage untargeted sequencing (i.e., single run whole genome sequencing data) can be fast and economical, and can deliver genome-wide polymorphism sensitivity in addition to the target-specific sequencing data. In addition, variants detected in the low coverage untargeted sequencing data can be used to identify known haplotype blocks and impute variants over the whole genome with or without targeted data.

Untargeted sequencing (e.g., whole genome sequencing) can determine the complete DNA sequence of the genome at one time. Untargeted sequencing (e.g., whole genome sequencing or the non-exonic portion of whole exome sequencing) can cover sequences of almost about 100 percent, or about 95%, of the sample's genome. In some cases, the untargeted sequencing (e.g., whole genome sequencing or non-exonic portion of the whole exome sequencing) can cover sequences of the whole genome of the nucleic acid sample of about or at least about 99.999%, 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 74%, 73%, 72%, 71%, 70%, 69%, 68%, 67%, 66%, 65%, 64%, 63%, 6%, 61%, 60%, 59%, 58%, 57%, 56%, 55%, 54%, 53%, 52%, 51%, or 50%.

Quality of NGS data and variant detection can be sensitive to conditions of sample library preparation. Negative effects can manifest as false positive and/or false negative allele detection, stochastic coverage, GC biases, poor library complexity, and lack of reproducibility. In clinical settings these can manifest in poor specificity, selectivity, positive predictive value (PPV), and negative predictive value (NPV).

Target-Specific Sequencing

Target-specific sequencing can be used as a method for generating sequencing data. Target-specific sequencing can be selective sequencing of specific genomic regions, specific genes, or whole exome sequencing. Non-limiting examples of the genomic regions include one or more polymorphisms, sets of genes, sets of regulatory elements, micro-deletions, homopolymers, simple tandem repeats, regions of high GC content, regions of low GC content, paralogous regions, degenerate-mapping regions, or a combination thereof. The sets of genes or regulatory elements can be related to one or more specific genetic disorders of interest. The one or more polymorphisms can comprise one or more single nucleotide variations (SNVs), copy number variations (CNVs), insertions, deletions, structural variant junctions, variable length tandem repeats, or a combination thereof.

In some cases, the target-specific sequencing data can comprise sequencing data of some untargeted regions. One example of the target-specific sequencing is the whole exome sequencing. Whole exome sequencing can be target-specific or selective sequencing of coding regions of the DNA genome. The targeted exome can be the portion of the DNA that translates into proteins, or namely exonic sequence. However, regions of the exome that do not translate into proteins can also be included within the sequence, namely non-exonic sequences. In some cases, non-exonic sequences are not included in exome studies. In the human genome there can be about 180,000 exons: these can constitute about 1% of the human genome, which can translate to about 30 megabases (Mb) in length. It can be estimated that the protein coding regions of the human genome can constitute about 85% of the disease-causing mutations. The robust approach to sequencing the complete coding region (exome) can be clinically relevant in genetic diagnosis due to the current understanding of functional consequences in sequence variation, by identifying the functional variation that is responsible for both mendelian and common diseases without the high costs associated with a high coverage whole-genome sequencing while maintaining high coverage in sequence depth. Other aspect of the exome sequencing can be found in Ng S B et al., “Targeted capture and massively parallel sequencing of 12 human exomes,” Nature 461 (7261): 272-276 and Choi M et al., “Genetic diagnosis by whole exome capture and massively parallel DNA sequencing,” Proc Natl Acad Sci USA 106 (45): 19096-19101.

### Sensitivity, Specificity, Accuracy, Coverage, and Uniformity

Quality of NGS data and variant detection can be sensitive to conditions of sample library preparation. Negative effects can manifest as both false positive and false negative allele detection, stochastic coverage, GC biases, poor library complexity and lack of reproducibility. In clinical settings these can manifest in poor specificity, selectivity, positive predictive value (PPV) and negative predictive value (NPV).

Assembled sequence reads from can be mapped aligned and variants called by latest version BWA/GATK using the Arvados platform through bioinformatics partners at Curoverse. Additional publicly available tools and custom analysis developed can be used to generate overall sequencing performance statistics for enrichment metrics, variant concordance and reproducibility, library complexity, GC bias, along with sequencing read depth, quality and uniformity. Tools for primer sequence trimming of amplicon reads can also be implemented. Variant calls can be processed through an automated bioinformatics decision tree under development with bioinformatics partner (Omicia).

The methods and systems disclosed herein for identifying a genetic condition in a subject can be characterized by having a specificity of at least about 50%. The specificity of the method can be at least about 50%, 53%, 55%, 57%, 60%, 63%, 65%, 67%, 70%, 72%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The specificity of the method can be at least about 70%. The specificity of the method can be at least about 80%. The specificity of the method can be at least about 90%.

In an aspect, provided herein is a method of identifying a genetic condition in a subject that gives a sensitivity of at least about 50% using the methods disclosed herein. The sensitivity of the method can be at least about 50%, 53%, 55%, 57%, 60%, 63%, 65%, 67%, 70%, 72%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The sensitivity of the method can be at least about 70%. The sensitivity of the method can be at least about 80%. The sensitivity of the method can be at least about 90%.

The methods and systems disclosed herein can improve upon the accuracy of current methods of identifying a genetic condition in a subject. The methods and systems disclosed herein for use of identifying a genetic condition in a subject can be characterized by having an accuracy of at least about 50%. The accuracy of the methods and systems disclosed herein can be at least about 50%, 53%, 55%, 57%, 60%, 63%, 65%, 67%, 70%, 72%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The accuracy of the methods and systems disclosed herein can be at least about 70%. The accuracy of the methods and systems disclosed herein can be at least about 80%. The accuracy of the methods and systems disclosed herein can be at least about 90%.

The methods and systems for use in identifying, classifying or characterizing a sample can be characterized by having a negative predictive value (NPV) greater than or equal to 90%. The NPV can be at least about 90%, 91%, 92%, 93%, 94%, 95%, 95.2%, 95.5%, 95.7%, 96%, 96.2%, 96.5%, 96.7%, 97%, 97.2%, 97.5%, 97.7%, 98%, 98.2%, 98.5%, 98.7%, 99%, 99.2%, 99.5%, 99.7%, or 100%. The NPV can be greater than or equal to 95%. The NPV can be greater than or equal to 96%. The NPV can be greater than or equal to 97%. The NPV can be greater than or equal to 98%.

The methods and/or systems disclosed herein for use in identifying, classifying or characterizing a sample can be characterized by having a positive predictive value (ITV) of at least about 30%. The PPV can be at least about 32%, 35%, 40%, 45%, 50%, 55%, 60%, 65%, 70%, 75%, 80%, 85%, 90%, 95%, 95.2%, 95.5%, 95.7%, 96%, 96.2%, 96.5%, 96.7%, 97%, 97.2%, 97.5%, 97.7%, 98%, 98.2%, 98.5%, 98.7%, 99%, 99.2%, 99.5%, 99.7%, or 100%. The PPV can be greater than or equal to 90%. The PPV can be greater than or equal to 95%. The PPV can be greater than or equal to 97%. The PPV can be greater than or equal to 98%.

The methods and systems disclosed herein for use in identifying, classifying or characterizing a sample can be characterized by having an error rate of less than about 30%, 25%, 20%, 19%, 18%, 17%, 16%, 15%, 14%, 13%, 12%, 11%, 10%, 9.5%, 9% 8.5%, 8%, 7.5%, 7% 6.5%, 6%, 5.5% 5% 4.5%, 4%, 3.5%, 3%, 2.5%, 2%, 1.5%, or 1%. The methods and systems disclosed herein can be characterized by having an error rate of less than about 1%, 0.9%, 0.8%, 0.7%, 0.6%, 0.5%, 0.4%, 0.3%, 0.2%, 0.1% or 0.005%. The methods and systems disclosed herein can be characterized by having an error rate of less than about 10%. The method can be characterized by having an error rate of less than about 5%. The methods, kits, and systems disclosed herein can be characterized by having an error rate of less than about 3%. The methods, kits, and systems disclosed herein can be characterized by having an error rate of less than about 1%. The methods, kits, and systems disclosed herein can be characterized by having an error rate of less than about 0.5%.

The methods and systems for use in identifying, classifying or characterizing a sample can be characterized by having coverage greater than or equal to 70%. The coverage can be at least about 70%, 75%, 80%, 85%, 90%, 91%, 92%, 93%, 94%, 95%, 96%, 97%, 98%, 99%, 99.2%, 99.5%, 99.7%, or 100%. The coverage can be greater than or equal to 70%. The coverage can be greater than or equal to 80%. The coverage can be greater than or equal to 90%. The coverage can be greater than or equal to 95%.

The methods and systems for use in identifying, classifying or characterizing a sample can be characterized by having a uniformity of greater than or equal to 50% (e.g. 50% of reads are within a 4× range of coverage). The uniformity can be at least about 50%, 55%, 60%, 65%, 70%, 75%, 80%, 85%, 90%, 91%, 92%, 93%, 94%, 95%, 96%, 97%, 98%, 99%, 99.2%, 99.5%, 99.7%, or 100%. The uniformity can be greater than or equal to 75%. The uniformity can be greater than or equal to 80%. The uniformity can be greater than or equal to 85%. The uniformity can be greater than or equal to 90%.

In one aspect, one or more polymerases can be added directly in a blood or DBS lysate sample for direct amplification.

In one aspect, the methods and systems have a turnaround time of less than 30 days. In some cases, the methods and systems have a turnaround time of less than, for example, 1, 2, 3, 4, 5, 6, 7, 0, 9, 10, 11, 12, 13, 14, 15, 16, 17, 18, 19, 20, 21, 22, 23, 24, 25, 6, 27, 28, 29, or 30 days. In some cases, the methods and systems have a turnaround time of less than, for example, 6, 12, 18, 24, 30, 36, 42, 48, 54, 60, 66, or 72 hours. In some cases, the methods and systems have a turnaround time of less than, for example, 1, 2, 3, 4, 5, 6, 7, 8, 9, or 10 weeks. Turnaround time can be defined as the amount of time taken from obtaining a sample of a subject to generating a result using the methods and systems disclosed herein.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### EXAMPLES

Methods and systems of the present disclosure can be applied to various types of newborn conditions.

### Example 1

### Screening for Newborns Using TNGS

Patient Samples

Validation specimens, unless stated otherwise, were obtained from patients with known causal mutations in the Amish and Mennonite populations examined at the Clinic for Special Children (CSC) in Strasburg, Pa. Specimens were collected under informed consent as part of diagnostic and research protocols approved by both the Lancaster General Hospital and the Western institutional Review Boards. In this cohort, the disease-causing mutations were initially characterized by traditional Sanger DNA sequencing and were blinded for this NGS study. The clinic provided diagnosis and management of patients with inherited metabolic and genetic diseases within Amish and Mennonite populations. Mutations in the Amish and Mennonites are not unique, but in some cases, they occur in higher frequencies than they do in the general population. The high incidence of disease and carrier cases can thus be used to validate the analytical test performance and genotype-phenotype concordance of new testing methodologies.

Sample Processing, Target Capture, and NGS

Briefly, isolated DNA was fragmented, barcoded with NGS library adapters, and incubated with oligonucleotide probes for DNA target capture, as outlined by the manufacturer (Roche Diagnostics, Indianapolis, Ind.), for all coding exons (SeqCap EZ Human Exome Library v2.0; 44-Mb target) or the NBDx targeted panel (SeqCap EZ Choice; up to 7-Mb target). Sequencing was performed with 2×75 bp HiSeq2500 rapid runs (Illumina, San Diego, Calif.). All NGS experiments were performed in research mode while keeping read depth and quality to mimic clinical grade metrics: >70% reads on target; >70× mean target base coverage; and >90% target bases covered >20×. An additional experiment used Nextera Rapid Capture (TruSight Inherited Disease; Illumina) for CYP21A2 testing on MiSeq.

NGS Analysis

Sequencing reads were aligned to hg19/GRCh37 using Burrows-Wheeler Aligner for short alignments, followed by Genome Analysis Toolkit v2.0 variant calling pipeline running on the Arvados platform (arvados.org). Opal 3.0 from Omicia (www.omicia.com) was used for variant annotation and analysis following guidelines of the American College of Medical Genetics.

ClinVar Site Coverage Calculation

ClinVar sites (www.ncbi.nlm.nih.gov/clinvar/) were determined by intersecting the NBDx tiled regions with the ClinVar track in the UCSC Browser (genome.ucsc.edu/) and removing duplicates to give a total of 6,215 unique ClinVar sites.

Results

TNGS Workflow Test Using in Silico NBS Gene Filter and Rapid Turnaround

New NGS workflows can be benchmarked against the traditional Sanger sequencing technology. CSC had previously identified more than 100 variants among the 120 different disorders identified at the clinic by Sanger sequencing, 32 of which were causal mutations for inborn errors of metabolism that are routinely screened by NBS. Ten (10) of the CSC patient samples identified by such benchmark methods were used to optimize WES and in silico filtering for detection of the causal genetic variants.

The WES workflow was initially tested with two disease cases that are common in the Amish and Mennonite populations, propionic acidemia and maple syrup urine disease type 1A, to identify attributes of filtering regimens and causal variants (Table 3), Simple filters for coverage, allele frequency, and pathogenicity reduced the number of variants in the WES samples from an average of 11,014 for exonic protein impact to 590. The in silico 126-gene NBS filter described in Table 4 reduced this to approximately four mutations, and the Sanger-validated causative homozygous mutations were identified. Thereafter, a blinded retrospective validation study was undertaken using eight randomly selected samples from the same population to benchmark results and demonstrate achievable turnaround times. The entire workflow from blood sample isolation through target capture, sequencing on a HiSeq 2500 in rapid run mode, informatics, and interpretation was parallel-processed within 105 hours for the eight WES samples (FIG. 1B). Capture performance data indicated that, on average, 95% of the target bases were covered at 10× read depth or more and, of the total mapped reads, 73% were in WES target regions. Using the 126-gene NBS in silico filter, the correct disorder and mutation, as previously validated by Sanger sequencing, were quickly identified by TNGS in all eight samples. One subject was suspected to be a compound heterozygote for PAH ((c.782 G>A/c.284-286del) OMIM 261600), indicative of phenylketonuria. This subject also had a heterozygous carrier mutation in MCCC2 (OMIM 609014), which is commonly present in the Amish population. A similar situation was found in the subject with 11-β-hydroxylase deficiency, whereby a carrier of the c.646 G>A mutation responsible for adenosine deaminase deficiency was identified. This mutation is also known to segregate in the Amish population. All other samples were found to be homozygous for the common mutations known to occur in the Amish and Mennonite populations (Table 3). Further, an alternate in silico gene filter representing 552 genes on the Illumina hereditary panel did not detect the mutations in IL7R and MTHFR (false-negative calls), which are genes that are not targeted in that panel.

TABLE 3
Application of in silico gene filtering
to blinded samples from WES. Disorders
detected by current expanded NBS programs

| | | Disorder |
|---|---|---|
| ID no. | Gene | OMIM no. |
| 1^{b-d} | ARG1 | 207800 |
| 2 | ASL | 207900 |
| 3 | GSS | 266130 |
| 4^{b-d} | OPLAH | 260005 |
| 5 | CPS1 | 237300 |
| 6 | ASS1 | 215700 |
| 7^{b-d} | SLC25A13 | 603859 |
| 8 | CBS | 236200 |
| 9^{a,c,d} | MTHFR | 236250 |
| 10^{b-d} | MTRR | 602568 |
| 11^{b-d} | MAT1A | 610550 |
| 12^{b-d} | OAT | 258870 |
| 13 | SLC25A15 | 238970 |
| 14^{a} | PAH | 261600 |
| 15^{b-d} | GCH1 | 233910 |
| 16^{b-d} | QDPR | 261630 |
| 17^{b-d} | PCBD1 | 264070 |
| 18^{b-d} | PTS | 261640 |
| 19^{b-d} | SPR | 612716 |
| 20^{a} | BCKDHA | 248600 |
| 21^{a} | BCKHDB | 248600 |
| 22^{b} | DBT | 248600 |
| 23 | DLD | 238331 |
| 24 | FAH | 276700 |
| 25^{b,c} | TAT | 276600 |
| 26^{b,c} | HPD | 276710 |
| 27 | HMGCL | 246450 |
| 28^{a} | GCDH | 231670 |
| 29^{b-d} | C7orf10 | 231690 |
| 30^{b-d} | ACAD8 | 604773 |
| 31 | IVD | 243500 |
| 32^{c,d} | ACADSB | 600301 |
| 33^{b-d} | MCCC1 | 210200 |
| 34^{c,d} | MCCC2 | 609014 |
| 35 | AUH | 250950 |
| 36 | TAZ | 302060 |
| 37 | OPA3 | 258501 |
| 38 | MUT | 251000 |
| 39^{b} | MMAA | 251100 |
| 40 | MMAB | 251110 |
| 41 | MMACHC | 277400 |
| 42^{b-d} | MMADHC | 277410 |
| 43^{b-d} | LMBRD1 | 277380 |
| 44^{b-d} | MTR | 156570 |
| 45^{b-d} | TCN2 | 613441 |
| 46^{b} | ACAT1 | 203750 |
| 47^{a,b} | PCCA | 282000 |
| 48^{a,b} | PCCB | 532000 |
| 49^{b} | HLCS | 253270 |
| 50^{b-d} | MLYCD | 248360 |
| 51 | ACADL | 609576 |
| 52 | ACADM | 201450 |
| 53^{c,d} | ACADS | 201470 |
| 54 | ACADVL | 201475 |
| 55 | CPT1A | 255120 |
| 56 | CPT2 | 255110 |
| 57^{b-d} | DECR1 | 222745 |
| 58 | HADH | 601609 |
| 59^{b} | SLC25A20 | 212138 |
| 60^{b} | SLC22A5 | 212140 |
| 61 | ETFA | 608053 |
| 62 | ETFB | 130410 |
| 63 | ETFDH | 231675 |
| 64 | HADHA | 143450 |
| 65 | HADHB | 143450 |
| 66^{a} | BTD | 253260 |
| 67^{a} | CFTR | 602421 |
| 68 | GALT | 606999 |
| 69^{b-d} | GALE | 606953 |
| 70^{b} | TALK1 | 604313 |
| 71 | HBB | 141900 |
| 72 | G6PD | 305900 |
| 73 | ADA | 102700 |
| 74 | RAG1 | 179615 |
| 75 | RAG2 | 179616 |
| 76^{b-d} | IL7R | 146661 |
| 77^{b-d} | IL2RA | 147730 |
| 78 | IL2RG | 308380 |
| 79^{b-d} | PTPRC | 151460 |
| 80^{b} | CD3E | 186830 |
| 81^{b} | CD3D | 186790 |
| 82 | DCLRE1C | 605988 |
| 83^{b} | NHEJ1 | 311290 |
| 84 | JAK3 | 600173 |
| 85^{b-d} | ZAP70 | 176947 |
| 86^{b} | LIG4 | 601837 |
| 87^{b-d} | PNP | 164050 |
| 88^{b-d} | LCK | 153390 |
| 89^{b-d} | DUOX2 | 606759 |
| 90^{b-d} | DUOXA2 | 612772 |
| 91^{b-d} | FOXE1 | 602617 |
| 92 | LHX3 | 600577 |
| 93^{b-d} | NKX2-1 | 600635 |
| 94^{b-d} | NKX2-5 | 600584 |
| 95^{b-d} | PAX8 | 167415 |
| 96 | POU1F1 | 173110 |
| 97 | PROP1 | 601538 |
| 98^{b-d} | TG | 188450 |
| 99^{b-d} | TPO | 606765 |
| 100^{b-d} | TRHR | 188545 |
| 101 | TSHB | 188540 |
| 102^{b-d} | TSHR | 603372 |
| 103^{b} | CYP11B1 | 610613 |
| 104^{b} | CYP17A1 | 609300 |
| 105 | CYP21A2 | 613815 |
| 106^{b} | HSD3B2 | 613890 |
| 107 | STAR | 600617 |

New conditions added to in silico filter

| | | |
|---|---|---|
| 108 | ALDOB | 612724 |
| 109^{a} | CTNS | 606272 |
| 110^{b-d} | AASS | 238700 |
| 111^{c,d} | HGD | 203500 |
| 112^{b-d} | HGMCS2 | 600234 |
| 113^{c,d} | SERPINA1 | 107400 |
| 114^{b-d} | SLC7A7 | 603593 |
| 115 | IDUA | 252800 |
| 116^{b} | IDS | 300823 |
| 117^{b-d} | GALNS | 612222 |
| 118 | GLB1 | 611458 |
| 119 | ARSB | 611542 |
| 120 | GUSB | 611499 |
| 121 | ATP7B | 606882 |
| 122 | GBA | 606463 |
| 123 | GAA | 606800 |
| 124 | GALC | 606890 |
| 125 | OTC | 311250 |
| 126 | NAGS | 608300 |

An in silico gene filter was developed that calls variants in the 126 genes relating to newborn diseases and the NBDx capture probe set that targets these same genes. 107 genes corresponding to diseases detected by current NBS biochemical assays in the United States. 19 supplemental genes that meet criteria set forth for inclusion in routine NBS but are currently not undertaken or lack a biochemical screening method. The corresponding OMIM identifiers are provided. The NBDx capture probe set targets 1.4 Mb covering the 126 NBS genes within a total 5.9 Mb target region.
^{a}Ten of the NBS genes include intronic coverage for variant determination similar to WGS. ^{b,c}Not covered on the Children's Mercy Hospital hereditary gene panel versions of 2011 and 2012, respectively. ^{6 d}Not covered on the 552 gene illumine hereditary panel (gene list at www.illumina.com/products/trusight_inherited_disease.ilmn).

TABLE 4
In silico filtering for 126 NBS genes in blinded whole exome sequencing cases

| | | | | | | | | <5% | | |
|---|---|---|---|---|---|---|---|---|---|---|
| | | | | | | | | MAF | 552- | 126- |
| | | | | | | | | ≧5 | Gene | Gene |
| | | | | | | | | Reads | hereditary | NBS |
| | | | | | | | | (PI, | filter* | filter* |
| | | Transcript | Protein | Zygo | | Exonic | Protein | OS | Total | Total |
| Sample | Gene | variant | variant | sity | Reads | variants | impact | ≧0.65) | (Hom) | (Hom) |
| Pipeline test | | | | | | | | | | |
| 28480 | BCKDHA | c.1312T>A | p.Tyr438Ans | Hom | 35 | 23069 | 11461 | 687 | 19(1) | 3(1) |
| 28839 | PCCB | c.1606A>G | p.Asn536Asp | Hom | 49 | 21681 | 10567 | 493 | 13(2) | 4(1) |
| Average | | | p. | | 42 | 22375 | 11014 | 590 | 16(2) | 4(1) |
| Ripid TNGS | | | | | | | | | | |
| S1 | IL7R | c.2T>G | p.Met1Arg | Hom | 198 | 24992 | 14080 | 531 | 19(0) | 2(1) |
| S3 | BTD | c.1459T>C | p.Trp487Arg | Hom | 74 | 25233 | 14269 | 599 | 18(2) | 3(1) |
| S4 | CYP11B1 | c.1343G>A | p.Arg448His | Hom | 57 | 24733 | 14051 | 604 | 24(7) | 7(1) |
| | ADA^{b} | c.646G>A | p.Gly216Arg | Het | 66 | | | | | |
| S5 | PAH | c.782G>A | p.Arg261Gln | Het | 33 | 25275 | 14363 | 729 | 30(1) | 6(0) |
| | PAH | c.284_286del | p.Ile95del | Het | 35 | | | | | |
| | MCCC2^{b} | c.295G>C | p.Glu99Gln | Het | 61 | | | | | |
| S6 | ACADM | c.985A>G | p.Lys329Glu | Hom | 101 | 24782 | 13909 | 585 | 19(4) | 3(2) |
| S7 | CFTR | c.1521_1523del | p.Phe508del | Hom | 43 | 25128 | 14142 | 646 | 25(6) | 6(2) |
| S9 | MTHFR | c.1129C>T | p.Arg377Cys | Hom | 92 | 25805 | 13968 | 567 | 24(2) | 4(1) |
| S10 | GALT | c.563A>G | p.Glu188Arg | Hom | 79 | 24743 | 14123 | 598 | 26(2) | 7(1) |
| | C7orf10^{b} | c.895C>T | p.Arg299Trp | Het | 70 | | | | | |
| Average | | | | | 76 | 25086 | 14113 | 607 | 24(3) | 5(1) |
| SD | | | | | 44 | 362 | 149 | 59 | 4(2.4) | 2(0.6) |

MAF minor allele frequency; NBS, newborn screening; OS, Omicia score; PI, protein impact; TNGS, targeted next-generation sequencing; WES, whole-exome sequencing.
Total number of WES variants, including those that have PI, after GATK2 variant processing is noted. For each, the Sanger-validated causative mutations and number of variants recovered using various filters are shown for WES samples.
^{a}126-Gene NBS filter (Table 1) and 552-gene hereditary filter6 include the specified genes filter plus ≧5 reads, <5% MAF, PI, and OS ≧0.65. Numbers in brackets are the same filters plus homozygosity.
^{b}Carrier mutation.

Validation of DNA Isolation from Minimally Invasive DBS and Small-Volume Whole Blood for TNGS

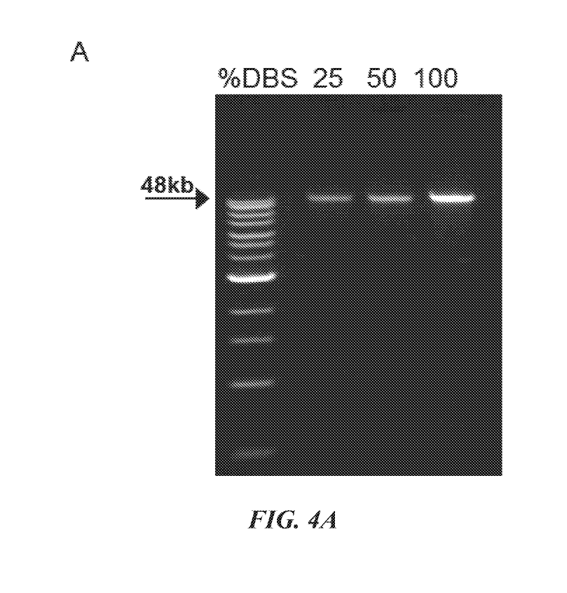

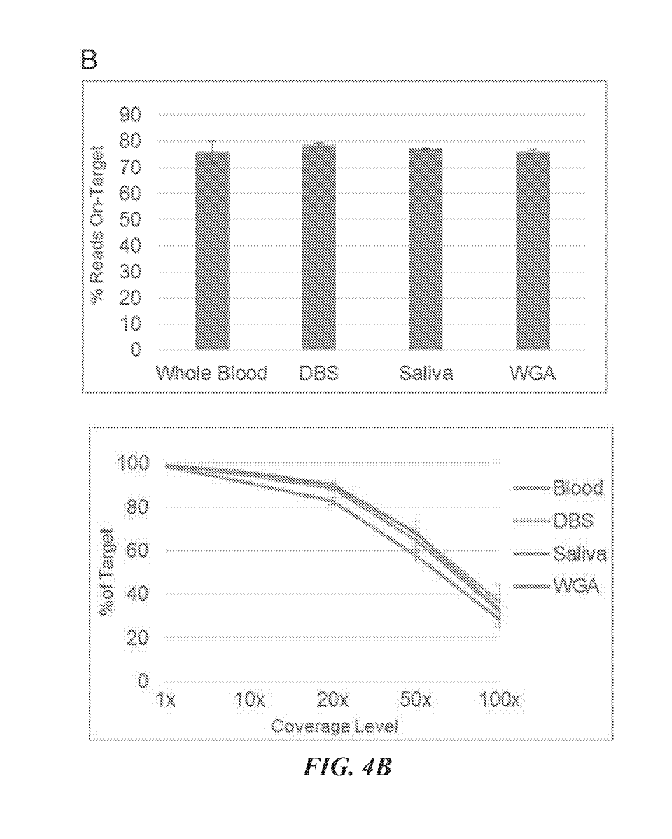

A robust and reproducible recovery of sufficient dsDNA from DBS for TNGS libraries which methods described herein, similarly high-quality TNGS performance of DNA isolated from DBS as compared with the standard 10 ml of whole blood and saliva was seen (FIGS. 4A and 4B). With a control sample set, protocols yielded ˜450 ng double-stranded DNA (dsDNA) from one-half of a single saturated spot from the DBS card, representing 25 μl blood (as measured by the dsDNA-specific QUBIT™ assay; Table 5). The SeqCap EZ capture method used here can require 200 ng dsDNA, and an additional 10 to 20 ng for quality-control measurements. Recent methods of NGS library construction can claim as little as 50 ng dsDNA for library construction (e.g., Nextera). Whole-genome amplification (WGA) could mitigate in cases of insufficient yield, and TNGS has been successfully performed with DNA from DBS after WGA using Repli-G Ultrafast (Qiagen). In comparisons of matched samples, the addition of WGA resulted in approximately 5% lower target region covered at read depths 10× to 100× (FIG. 49), yet concordance remained near 100% across approximately 80 variants.

TABLE 5
DNA Isolation from Biological Specimens.

| | | | # | | | |
|---|---|---|---|---|---|---|
| | Sample | | Isolations | dsDNA | Molecular | |
| Source | Volume | Protocol | (N) | Yield^{a} | Weight^{b} | qPCR^{c} |
| Small Volume Blood | 25 μl | QiaAmp | 5 | 775 ± 168 ng | >50 Mb | <1 ΔCt |
| DBS | 6 punches | OiaAmp | 16 | 440 ± 73 ng | >50 Mb | <1 ΔCt |
| | (equals ~25 μl) | | | | | |
| | | GenSolve | 7 | 413 ± 116 ng | >50 Mb | <1 ΔCt |
| | | Charge | 4 | 116 ± 24 ng | >50 Mb | >1 ΔCt |
| | | Switch | | | | |
| Saliva | 250 μl | QiaAmp | 5 | 1684 ± 344 ng | >50 Mb | <1 ΔCt |

Newborn-Specific Targeted Gene Panel (NBDx) Capture and NGS Performance

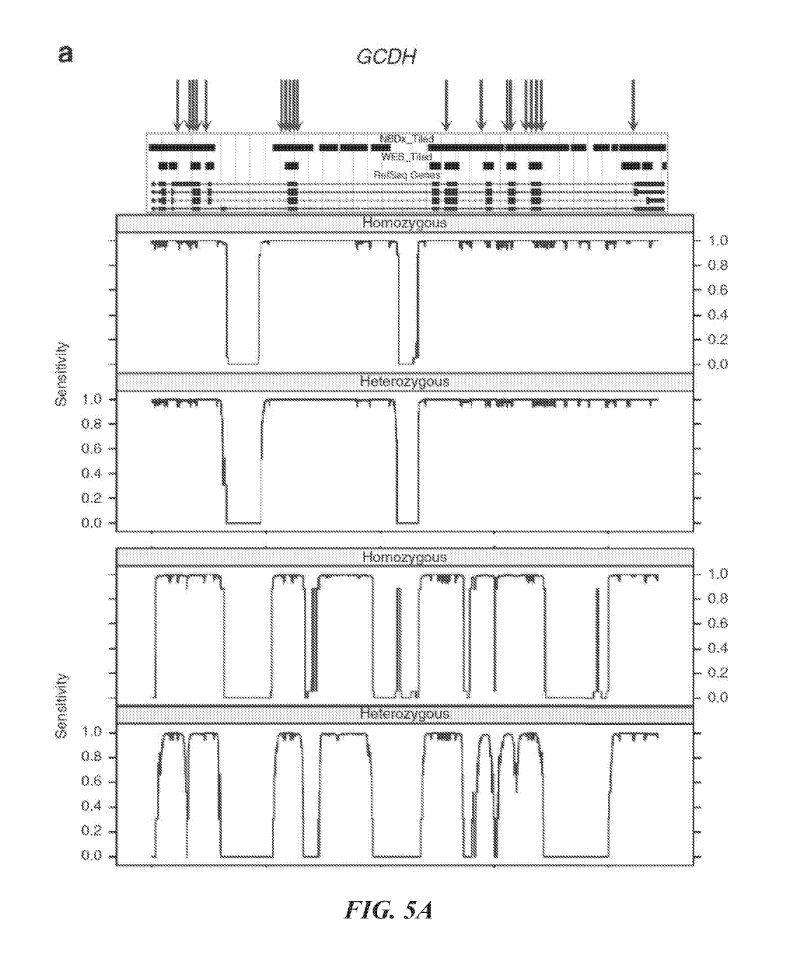

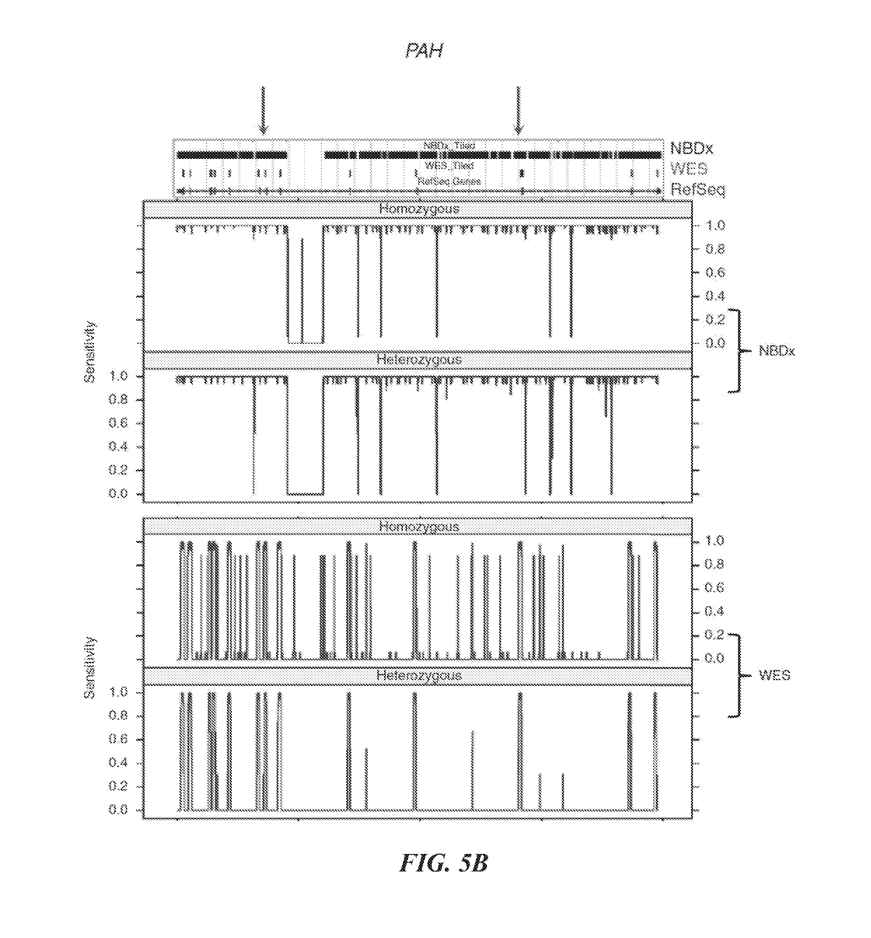

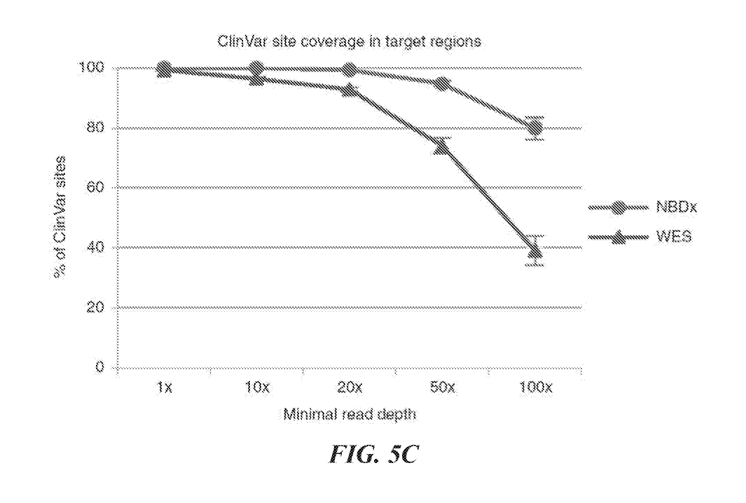

To measure NBDx gene panel performance, 36 clinical samples that had mutations for metabolic diseases from the Amish and Mennonite populations were tested (Table 1 and Table 6). Eight samples from this set were common with those of the WES analysis performed earlier. All samples were previously charactetized by Sanger sequencing but were anonymized and thus interpreted in a blinded fashion regarding the disorder and mutation present. It was ultimately revealed that the samples had causative mutations in 18 separate disease-related genes. Eleven samples in the set showed 19 different mutations spanning across the glutaric acidemia type I gene, GCDH (arrows in FIGS. 5A and 5B).

TABLE 6
Tabulation of Disease Positive Calls.

| | Adrenal | | | | | | | |
|---|---|---|---|---|---|---|---|---|
| | Hyperplasia | | | | | Glutaric | | |
| | (CYP11B1, | Biotinidase | Cystic | | Galacto- | Aciduria | Homo- | |
| | CYP21A2, | Deficiency | Fibrosis | GA-1 | semia | III | cystinuria | MCAD |
| | HSD3B2) | (BTD) | (CFTR) | (GCDH) | (GALT) | (c7orf10) | (MTHFR) | (ACADM) |
| #Expected | 4 | 2 | 1 | 11 | 2 | 1 | 1 | 3 |
| Filtering | 2 | 2 | 0 | 9 | 2 | 1 | 1 | 1 |
| Only | | | | | | | | |
| (assuming | | | | | | | | |
| Hets are in | | | | | | | | |
| trans) | | | | | | | | |
| Filtering | 2 | 2 | 0 | 1 | 2 | 1 | 1 | 1 |
| Only | | | | | | | | |
| (without in | | | | | | | | |
| trans | | | | | | | | |
| assumption) | | | | | | | | |
| Filtering | 2 | 2 | 1 | 9 | 2 | 1 | 1 | 1 |
| Only | | | | | | | | |
| (after | | | | | | | | |
| correcting | | | | | | | | |
| annotation) | | | | | | | | |
| With | 2 | 2 | 1 | 11 | 2 | 1 | 1 | 3 |
| Clinical | | | | | | | | |
| Phenotype | | | | | | | | |
| | | | | | | # | | |
| | | | | | | Samples | | |
| | 3-MCC | MSUD | | | | with | | |
| | Defi- | (DBT, | | | | Correct | Undetermined | Total |
| | ciency | BCKDHA, | PKU | SCID | Tyrosinemia | Disease | (or | # |
| | (MCCC2) | BCKDHB) | (PAH) | (IL7R) | (HPD) | Positive | Carrier-only)^{a} | Samples |
| #Expected | 2 | 3 | 1 | 1 | 2 | n/a | 2 | 36 |
| Filtering | 1 | 2 | 1 | 1 | 2 | 25 | 11 | 36 |
| Only | | | | | | | | |
| (assuming | | | | | | | | |
| Hets are in | | | | | | | | |
| trans) | | | | | | | | |
| Filtering | 1 | 0 | 0 | 1 | 1 | 13 | 23 | 36 |
| Only | | | | | | | | |
| (without in | | | | | | | | |
| trans | | | | | | | | |
| assumption) | | | | | | | | |
| Filtering | 2 | 2 | 1 | 1 | 2 | 27 | 9 | 36 |
| Only | | | | | | | | |
| (after | | | | | | | | |
| correcting | | | | | | | | |
| annotation) | | | | | | | | |
| With | 2 | 3 | 1 | 1 | 2 | 32 | 4 | 36 |
| Clinical | | | | | | | | |
| Phenotype | | | | | | | | |

Calls across the 13 disorders represented in the sample set run with NBDx. Number of samples per disorder based on known phenotypes and previous Sanger sequencing are given. The number of disease positive calls are given for various scenarios: (1) Variant Filtering Only, with an assumption of heterozygous calls being in trans. (2) Variant Filtering Only, without assuming heterozygous are in trans. These can require further confirmation if the samples did not have a priori Sanger data. Although it was not known to those performing NGS and variant calling, samples had been selected to carry multiple mutations, including the majority of GCDH, in order to maximize testing of variant detection across the gene. (3) Variant Filtering Only, after discovered mis-annotations were corrected in the database (assuming heterozygous are in trans) and (4) Variant Filtering plus Clinical Phenotype. Corrections made with clinical information are given per sample in Table 1. aUndetermined includes samples with only carrier status identified, or ambiguity in ability to call (e.g. VUS or multiple genes with >1 heterozygous mutation). Two of the blinded samples were carrier-only. For these, a correct call can be the same as no disease status identified (Undetermined).

Next, NBDx for capture enrichment performance was compared against WES. NBDx captures were processed at 20 samples per HiSeq2500 lane in rapid run mode, as compared with four samples for WES (Table 7). The average reads on target were approximately twofold higher for NBDx compared with WES (151× vs. 88×) because of focused sequencing combined with a higher on-target specificity relative to WES (87% vs. 73%). Because read depth can be a good predictor of variant detection (sensitivity), it was used to identify regions that are undercovered, i.e., less than 13 reads (FIGS. 5A & 5B). Sensitivity plots for GCDH and PAH across chromosomal positions were generated for WES or NBDx, as previously described by Meynert et al. As expected, compared with NBDx, WES had lower sensitivity because of lack of intronic probe coverage in PAH and GCDH.

TABLE 7
Sequencing and Enrichment Statistics for the NBDx and WES Samples.

| | | Raw | Reads | Reads | %Target | %Target | %Target | %Target | %Target | | |
|---|---|---|---|---|---|---|---|---|---|---|---|
| | | Reads | Mapped | On-Target | Covered | Covered | Covered | Covered | Covered | Average | |
| ID | Panel | (Millions) | (Millions) | (Millions) | 1X | 10X | 20X | 50X | 100X | Reads | Specificity |
| S1 | WES | 90.0 | 95.8 | 65.8 | 99.2 | 95.2 | 89.9 | 69.1 | 37.4 | 99 | 76.7 |
| S3 | WES | 84.5 | 79.8 | 61.4 | 99.4 | 95.4 | 89.9 | 67.6 | 34.2 | 97 | 76.9 |
| S4 | WES | 95.6 | 91.2 | 70.9 | 99.4 | 95.7 | 90.9 | 71.9 | 41.2 | 108 | 77.7 |
| S5 | WES | 49.4 | 37.4 | 20.7 | 99.6 | 92.3 | 76.2 | 29.9 | 5.8 | 48 | 55.3 |
| S6 | WES | 93.0 | 89.1 | 69.0 | 99.3 | 95.1 | 89.6 | 69.4 | 39.1 | 102 | 77.4 |
| S7 | WES | 67.7 | 58.3 | 38.2 | 99.5 | 95.3 | 87.6 | 54.4 | 17.4 | 72 | 65.4 |
| S9 | WES | 76.8 | 72.7 | 56.1 | 99.3 | 94.7 | 88.2 | 63.3 | 29.5 | 89 | 77.2 |
| S10 | WES | 75.1 | 72.0 | 56.5 | 99.2 | 93.9 | 86.9 | 62.2 | 29.7 | 88 | 78.5 |
| S1 | NBDx | 17.5 | 17.0 | 14.7 | 97.0 | 94.3 | 92.2 | 87.4 | 74.7 | 147 | 86.5 |
| S3 | NBDx | 17.1 | 16.4 | 14.0 | 97.0 | 94.6 | 92.6 | 87.8 | 73.6 | 149 | 85.6 |
| S4 | NBDx | 16.2 | 15.8 | 13.7 | 97.2 | 94.3 | 92.1 | 86.6 | 71.3 | 148 | 86.7 |
| S5 | NBDx | 11.5 | 9.3 | 7.0 | 96.7 | 92.4 | 89.5 | 68.9 | 24.4 | 81 | 75.4 |
| S6 | NBDx | 16.3 | 15.9 | 13.7 | 97.2 | 94.1 | 91.8 | 86.5 | 71.2 | 150 | 86.6 |
| S7 | NBDx | 13.8 | 13.3 | 11.7 | 97.0 | 94.4 | 92.2 | 86.3 | 63.9 | 134 | 88.2 |
| S9 | NBDx | 16.1 | 15.6 | 13.5 | 97.0 | 94.1 | 91.8 | 86.3 | 70.3 | 140 | 86.5 |
| S10 | NBDx | 15.9 | 15.4 | 13.4 | 97.1 | 94.2 | 92.1 | 86.6 | 70.6 | 142 | 86.5 |
| S11 | NBDx | 13.5 | 13.2 | 11.7 | 97.2 | 94.6 | 92.4 | 86.2 | 64.3 | 133 | 88.7 |
| 4963 | NBDx | 18.6 | 17.9 | 15.5 | 97.5 | 94.7 | 92.8 | 87.9 | 76.5 | 161 | 86.5 |
| 6810 | NBDx | 18.7 | 18.0 | 15.6 | 97.5 | 95.0 | 93.1 | 88.3 | 77.2 | 160 | 86.5 |
| 7066 | NBDx | 17.6 | 17.0 | 14.7 | 97.2 | 94.6 | 92.7 | 87.7 | 75.3 | 154 | 86.5 |
| 7241 | NBDx | 18.1 | 17.6 | 15.6 | 97.5 | 95.4 | 93.8 | 89.3 | 78.5 | 158 | 88.8 |
| 7656 | NBDx | 18.3 | 17.6 | 15.3 | 97.4 | 94.6 | 92.6 | 87.7 | 75.9 | 166 | 86.6 |
| 7901 | NBDx | 20.7 | 20.0 | 17.3 | 97.4 | 94.9 | 93.1 | 88.7 | 79.5 | 173 | 86.8 |
| 7912 | NBDx | 18.1 | 17.5 | 15.2 | 97.2 | 94.5 | 92.6 | 87.7 | 75.7 | 160 | 86.8 |
| 10241 | NBDx | 19.6 | 19.0 | 16.4 | 97.5 | 94.9 | 93.1 | 88.5 | 77.9 | 163 | 86.3 |
| 10642 | NBDx | 23.1 | 22.3 | 19.2 | 97.2 | 94.8 | 93.2 | 89.4 | 82.2 | 176 | 86.1 |
| 13925 | NBDx | 15.4 | 15.0 | 13.4 | 96.9 | 94.5 | 92.4 | 87.0 | 70.9 | 145 | 89.4 |
| 14691 | NBDx | 16.9 | 16.4 | 14.1 | 97.4 | 94.7 | 92.7 | 87.4 | 73.5 | 148 | 86.3 |
| 16622 | NBDx | 19.0 | 18.4 | 15.9 | 97.4 | 95.1 | 93.3 | 88.6 | 77.5 | 172 | 86.3 |
| 19283 | NBDx | 14.2 | 13.8 | 12.3 | 97.0 | 94.3 | 92.0 | 85.7 | 66.2 | 138 | 89.2 |
| 21901 | NBDx | 17.7 | 17.1 | 14.7 | 97.4 | 94.7 | 92.7 | 87.7 | 75.5 | 155 | 86.1 |
| 22785 | NBDx | 20.1 | 19.4 | 16.7 | 97.6 | 95.1 | 93.3 | 89.0 | 79.9 | 173 | 86.0 |
| 23275 | NBDx | 14.8 | 14.5 | 12.9 | 97.2 | 94.7 | 92.6 | 86.8 | 69.2 | 133 | 88.9 |
| 23279 | NBDx | 14.9 | 14.5 | 12.9 | 97.3 | 94.8 | 92.7 | 86.8 | 69.7 | 142 | 88.8 |
| 25875 | NBDx | 18.7 | 18.1 | 15.7 | 97.5 | 95.1 | 93.3 | 88.5 | 77.1 | 159 | 86.3 |
| 26607 | NBDx | 17.2 | 16.7 | 14.5 | 97.3 | 94.8 | 92.8 | 87.4 | 74.5 | 150 | 86.6 |
| 27244 | NBDx | 13.8 | 13.4 | 11.9 | 97.2 | 94.2 | 91.8 | 85.5 | 64.5 | 130 | 88.9 |
| 28907 | NBDx | 17.8 | 17.3 | 15.4 | 97.4 | 95.1 | 93.1 | 88.3 | 76.6 | 159 | 88.6 |
| 29351 | NBDx | 20.4 | 19.8 | 17.0 | 97.7 | 95.3 | 93.7 | 89.4 | 80.3 | 170 | 86.3 |
| 31206 | NBDx | 18.4 | 17.8 | 15.5 | 97.1 | 94.6 | 92.7 | 87.8 | 75.9 | 157 | 86.7 |
| WES | Average | 79 | 73.3 | 55 | 99 | 95 | 87 | 61 | 29 | 88 | 73 |
| | Stdev | 15 | 18.1 | 17 | 0 | 1 | 5 | 14 | 12 | 19 | 8 |
| NBDx | Average | 17 | 16.6 | 14 | 97 | 95 | 93 | 87 | 72 | 151 | 87 |
| | Stdev | 2 | 2.5 | 2 | 0 | 1 | 1 | 3 | 10 | 18 | 2 |

Samples were run using Nimblegen SeqCap capture and HiSeq 2500 sequencing, in sets of 4 samples for WES and 20 samples for NBDx. As measured in Picard, PCR duplication rates were ˜5% for WES and ˜7% for NBDx. An additional 10 samples with mutations spanning PAH and 5 GCDH samples were run from archival DBS stored at room temperature for over 10 years. While mutations were able to be called, the majority of these samples were highly degraded, made use of whole genome amplification and did not have a priori Sanger data and as such are not included here.

The increased average sequencing depth in NBDx demonstrated that fewer targeted regions would fall below stringent valiant calling thresholds. This was shown in coverage of approximately 6,215 ClinVar sites common to both WES and NBDx tiled regions, which measured call coverage in regions of clinical relevance that can be monitored in every sample in real time (FIG. 5C). Furthermore, while both NBDx and WES started with more than 99% at 1× coverage, disparities began to show at 10× coverage; by 1.00× coverage, NBDx maintained 80% ClinVar coverage, but WES significantly declined to 39%. At 10× coverage, NBDx achieved close to 99.8% coverage, and at 1× coverage it achieved 99.99% coverage. Heterozygous calls up to one-sixth proportion were called (observed as 18 reads out of 120 total reads for this variant in NBDx) was empirically determined, by pooling samples and by allele dilution of rare pathogenic variants (e.g., GCDH (c.1262 C>T)).

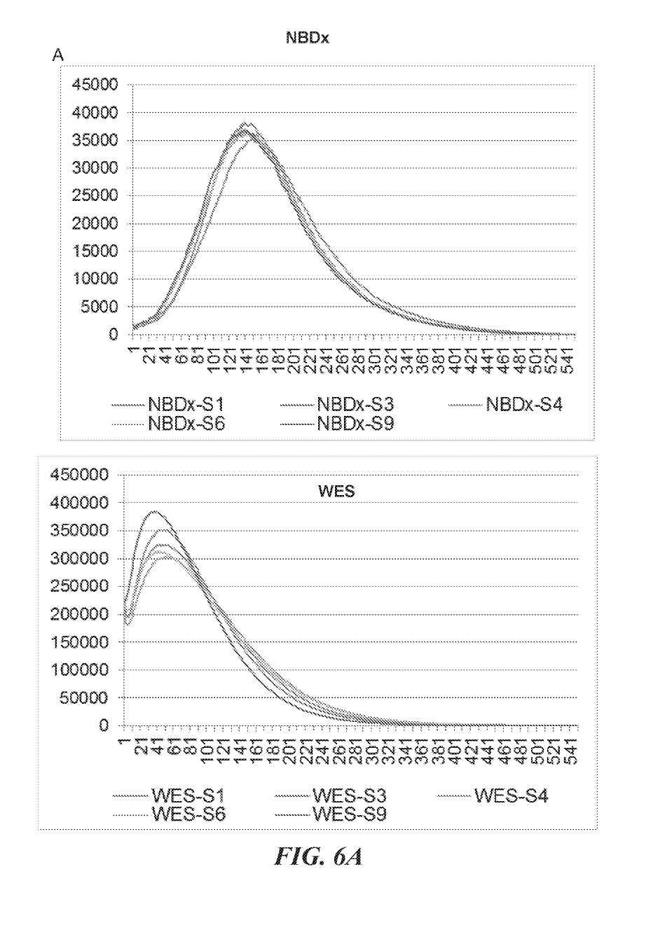

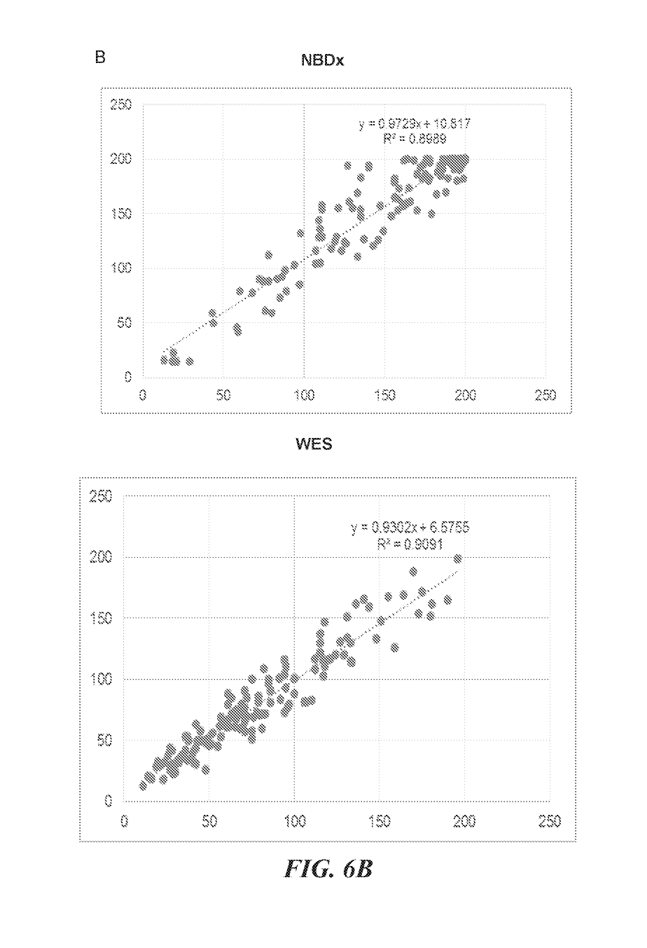

To assess uniformity or relative abundance of different targeted regions, base distribution coverage was compared. Good uniformity was obtained on NBDx data sets, but WES data showed significant skew toward low coverage, which is likely to reduce confidence on zygosity calls (FIG. 6A). To assess reproducibility, comparisons were performed for coverage depth at variant positions across matched data set pairs resulting from independent sample preparation and sequencing. The analysis suggested that DBS, 25 μl whole blood and saliva produced a similar proportion of calls with a high agreement (Pearson correlation coefficient=0.9) between replicates (FIG. 6B). Another aspect of reproducibility measured is the variability of coverage between runs in tiled regions. For 12 samples, the portion of the targeted region was sequenced with sufficient coverage to achieve 95% sensitivity for heterozygous calls (>13 reads) The maximum value per region was designated as 1. The tiled regions, for which at least one sample had a value less than 1, are shown in Table 2. From comparisons across 4 to 20 unrelated TNGS samples and a simple statistic (Z-scoring), highly variable regions such as homozygous intronic deletions in PCCB between exons 10 and 11 were detected.

### NGS Genotype Call Concordance

To assess the genotype concordance, NGS genotype calls were compared to a priori-generated Sanger sequencing calls from the 36 subjects at CSC. The variations ranged across a variety of mutation types, including nonsynonymous variations, indels, stop gained, and intronic/splice site variations (Table 1 and Table 6). Concordance of disease calls based on NGS genotypes was determined according to two scenarios. The first was fully blinded to the condition present and only the NGS variant data were used to classify the genotype and assignment to a disease, whereas in the second scenario a description of the clinical phenotype was available to optimize the genotype call. Two damaging heterozygotes variants in the same disease gene were preliminarily assumed to be in trans until confirmation could be obtained from the de-blinded data. In patients, phasing of such haplotypes can be performed through Sanger sequencing of parents after NGS.

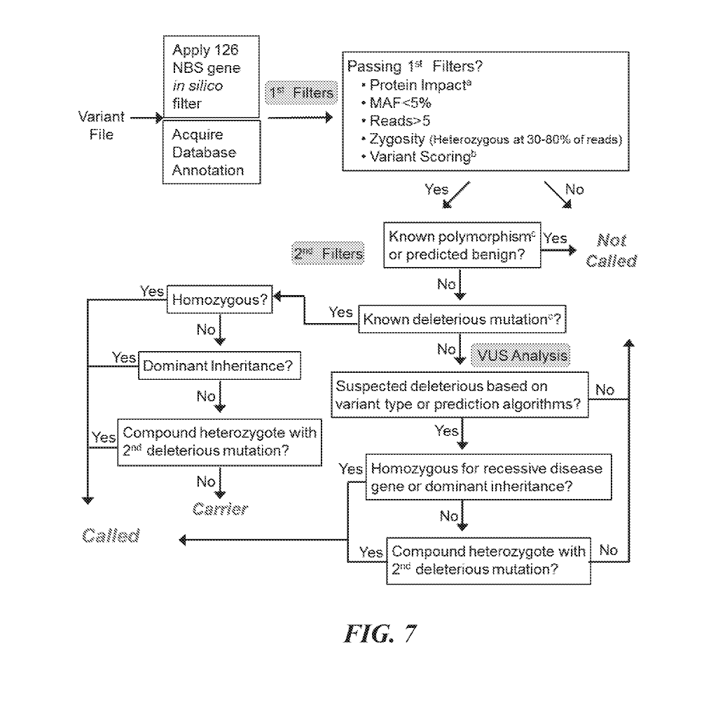

Using NGS genotype calls, preliminary disease calls in 27 out of 36 cases blindly (75%) were able to be made, suggesting difficulty of correctly classifying disease variants without clinical phenotype information, Complications (as noted in Table 1) included the following: (i) inability to distinguish causal variants from other mutations, either dominant or variants of unknown significance (VUS) with a predicted “damaging” classification; (ii) variant calling errors that were found on de-blinding for clinical phenotype, but, once corrected, these cases were processed through a standard filtering regimen (FIG. 7); (iii) no gene coverage (see CYP21A2 below); and (iv) compound heterozygotes with an intronic second mutation, which can require additional phenotype information. Clinical description plus a heterozygous damaging mutation in a disease-related gene enabled efficient intronic analysis within the same gene. Samples 9226 and 14691 had a combination of intronic mutations and heterozygosity in multiple genes.

A re-analysis with clinical summaries confirmed correct identification of mutations in seven additional disease or carrier cases, whereas two disease cases remained undetermined (ID 21901 and 27244) because the disease gene CYP21A2 was not targeted because of high pseudogene homology; however, false-positive calls were not made on these samples. A separate capture using the Illumina hereditary panel, that included CYP21A2, also failed to map the correct call. Two of the seven samples were carrier-status only (ID 23275 and 30221). Thus, with clinical phenotype, correct classification was obtained for 32 out of 34 disease cases (94.12%, 95% confidence interval, 80.29%-99.11%).

### Example 2

### Screening for Congenital Non-Syndromic Genetic Hearing Loss in Newborns Using NGS

Patient Samples

The specimens were collected under informed consent as part of diagnostic and research protocols approved by the Medical College of Virginia and the protocol was reviewed by the Western Institutional Review Board and considered as exempt status. DNA and biospecimens to validate the methodology were obtained from patients with known mutations. The disease causing mutations were initially characterized by traditional Sanger DNA sequencing. All individuals have profound sensory-neural hearing loss. Ethnic background is mainly Caucasian, a few are of Asian and African American decent. 95% of probands are from a multiplex family, and 5% are from a simplex family. Of the multiplex probands, 40% are from a deaf by deaf parental mating with all deaf children.

Patients DNA was targeted and enriched on hybrid-capture platforms (Roche Nimblegen SeqCap EZ Human Exome Library v2.0 or SeqCap EZ Choice for the targeted panel), and subsequently sequenced on the Illumina Hi-Seq 2000/2500 and analyzed using custom bioinformatics tools. Briefly, isolated DNA was fragmented mechanically for library adaptation, denatured, and incubated with oligonucleotide probes for hybrid-capture as outlined by the manufacturer. The Whole Exome Sequencing (a targeted sequence enrichment approach) has been described previously (Hodges et al. 2009, Ng et al. 2009) and was used for benchmark studies. Tens of thousands of oligonucleotide probes were utilized to enrich for the genomic DNA regions of entire coding exons (the 44.1 Mb Exome) or the targeted panel including. Following Hi-Seq 2000 or 2500 rapid run mode, the resulting sequencing reads were aligned to the reference genome (hg19/GRCH build 37). Following variant calling, the data was analyzed with a comprehensive genome interpretation software, Opal (Omicia, Emeryville, Calif.), to identify the correct disease variants for each sample specimen. In detail, the FASTQ files from the Hi-Seq2500 machine were processed with a pipeline running on the Arvados platform (arvados.org) that used the BWA aligner and the GATK toolkit for variant calling. Additionally, FASTQ files for Exomes were processed with the Real Time Genomics Variant 1.0 software, which includes a proprietary alignment and Bayesian variant calling algorithm and processes Exomes. The variant files were then uploaded into the Omicia Opal system for review and interpretation to identify the disease causing variant. In silico filter tools available within Omicia's Opal were used for gene set selection and for comparison with a variety of mutation and human variation databases (Clinvar, OMIM, HGMD). These tools were used to determine the pathogenicity of each variant by either previous knowledge in known mutation databases or by molecular impact as calculated by these prediction algorithms. The genes with mutations that had protein impact and were low frequency (less than 5% in the general population) were readily identified. Opal pre-classifies each variant in pathogenicity classes such as pathogenic, likely to be pathogenic, or benign such as suggested and published by the American College of Medical Genetics. The algorithms were reviewed and customized for clinical interpretation in conjunction with disease group experts and clinical consultants to identify variants. It was demonstrated that with Exomes the method can parallel process 8 to 10 Exomes per 105 hours; it can process several hundred per week on a TNGS panel. On some of the amplicon methods being tested, an even shorter turnaround times can be achieved and therefore higher throughput per week.

Establish DNA Purification Methods for DBS and Evaluate Against Whole Blood and Saliva Samples for TNGS

DNA Isolation Techniques Used and Developments

As studies began a technical challenge in some cases was to obtain sufficient yield and quality of double-stranded DNA from DBS. However, some examples can be a minimally acceptable baseline quantity, and the GenSolve Reagent coupled with Qiagen columns was used as a benchmark technique from which to further examine two other approaches: 1) ChargeSwitch Forensic magnetic bead based protocol, as a candidate for higher throughput isolation, and 2) The newer QiaAmp Micro Blood kit, using the lysis reagents included in the kit instead of GenSolve Protease Reagent. Modifications to the QiaAmp protocol were made to maximize DNA recovery and to meet concentration requirements for NGS library construction. Specifically, lysis reactions were scaled to allow more material going onto a single column and a multi-step elution scheme was utilized for recovery in smaller volume. Whole blood was tested by collection in lavender (EDTA) tubes and isolation of 25-50 μl using either: 1) QiaAmp Micro Blood kit, or 2) Modifying the Blood DNA Isolation kit from Roche, a protein precipitation method, and adapt it for use with the smaller volumes instead of the published minimum of 3 cc. Saliva samples were collected using the ORAGENE™ OG-575 or OC-100 devices and similarly tested by two methods: 1) QiaAmp Micro Blood kit with protocol modification for sample volume and 2) PrepIT L2P Reagent, a column-free protein precipitation method from DNA Genotek. Initial analysis of DNA isolation protocols was performed across sample types from at least two individuals.

TNGS specific characterization of isolated DNA:DNA isolation protocols need specific consideration for downstream use in NGS library production. Yield is one consideration, although it can be successfully compensated by whole genome amplification (WGA) methods. Beyond yield, DNA integrity or lack thereof can be important. Many of the current DNA isolation protocols, especially for DBS, were developed for direct use in amplification-driven applications (such as qPCR). In PCR, the DNA is denatured for primer annealing so either single- or double-stranded DNA or partially degraded DNA samples can serve as templates. Moreover, boiling of the DBS, or a simple alkaline wash, can be sufficient to provide the DNA input in such assays. However, in NGS, DNA library construction can be driven by either ligation or transposition, both of which can make use of a double-stranded DNA substrate, to attach adapters for sample barcoding, amplification and sequencing priming. A high quality and samples free from inhibitors can be accomplished, as these can have negative performance effects on the downstream enzymatic steps of library production, and other sample contaminants (e.g., RNA, non-human sequences such as from microbiota). Inhibitors can come from blood card impurities, the EDTA preservative in whole blood, and protein components including hemoglobin from blood itself.

As summarized in Table 8, the isolated DNA was examined by several assays: 1) QUBIT™ (Life Technologies) dsDNA specific assay for yield. 2) Agarose gel for intact quality of the DNA at high MW and RNA removal 3) Spectrophotometry for purity from RNA and other impurities (OD260/230 and OD 260/280). 4) qPCR inhibition assay for DNA purity from enzymatic inhibitors (the SPUD assay, Nolan 2006, uses ΔCt analysis of an artificial sequence spiked into the isolated DNA) and 5) qPCR of bacterial 16S rRNA genes for purity from contaminating microbial sequences (ORAGENE™ Bacterial DNA Assay, PD-PR-065). Examples of PCR inhibition and Agarose gel QC are shown in FIG. 4A.

TABLE 8
DNA isolation from bio-specimens.

| Source | | | | | | | |
|---|---|---|---|---|---|---|---|
| (n = 4-12 | Sample | | dsDNA | | | | |
| each) | Volume | Protocol | Yield | High MW | PCR | RNA | Bacteria |
| Small | 25 μl | QiaAmp | 776 ± 118 ng | >50 Mb | <1 ΔCt | N | N |
| Volume | | Roche Blood | | | | | |
| Blood | | DNA | 710 ± 76 ng | variable | <1 ΔCt | N | N |
| DBS | ½ spot (equal | QiaAmp | 512 ± 56 ng | >50 Mb | <1 ΔCt | N | N |
| | to ~25 μl) | GeneSolve | 413 ± 116 ng | >50 Mb | <1 ΔCt | N | N |
| | | Charge Switch | 116 ± 24 ng | >50 Mb | <1 ΔCt | N | N |
| Saliva | 250 μl | QiaAmp | 1684 ± 507 ng | >50 Mb | <1 ΔCt | N | Y |
| | | L2P | 1400 ± 495 ng | variable | <1 ΔCt | Y | Y |

High quality dsDNA was able to be obtained, free of RNA and inhibitors from all three sample sources. Additionally, all three sources gave sufficient yield for downstream use in the NGS application. Good purifications in terms of yield and the other quality metrics was obtained using modified protocols with the QiaAmp Blood Micro kit. For blood isolations, a theoretical full DNA recovery of 900-1800 ng/25 ml sample (based on average WBC counts and molecular weight of a diploid human genome) was calculated and found to recover dsDNA approaching that range. Isolation from DBS had lower yields, presumably due to lack of recovery from the paper cellulose card. A head-to-head comparison of 25 ml whole blood spotted in triplicate directly from the EDTA collection tube and stored dry for 1 day gave a dsDNA yield approximately half that recovered from the original liquid sample stored at 4° C. (419±32 ng for DBS vs 881±43 ng for whole blood). However, blood cards can be easily shipped and even after storage for long periods of time can give dsDNA yields similar to the fresh spots (Sjóholm et al. 2007 and see below). More than 1 mg dsDNA was also obtained for each saliva sample, albeit with bacterial contamination estimated to range from 10-30% based on qPCR results (see TNGS results for more detail on the consequences of bacterial DNA in saliva samples). However, in TNGS bacterial sequences are not selected for and therefore removed (see later section for discussions). The protein precipitation protocols, while in some cases not requiring the use of columns, can be more cumbersome due to a subsequent requirement for ethanol precipitation and had more variability for protein precipitate carry-over into the final DNA (3 of 8) and had a high percentage of DNA damage (4 of 8). The magnetic-bead based Charge Switch protocol suffered from both reduced yield of DNA and presence of inhibitors. However, other systems with higher capacity beads and improved washing regimens (Promega, Beckman Coulter) can provide alternate avenues for operational scale-up.

Hybrid Capture Performance in Exome Sequencing and Variant Detection Across Sample Types with an in Silico Hearing Loss Panel

A further consideration for use of DBS and other sample types in a TNGS approach is ensuring maintenance of coverage and accurate variant calling. DBS-derived DNA was Exome sequenced and the detected SNPs compared to DNA from alternate specimen types (whole blood, saliva and WGA of DBS-derived DNA; n=2-7 of each sample type). As shown in FIG. 4B, DBS and Saliva are similar to the more conventional whole blood isolation for both % reads on target (a measure of overall capture performance) and % of target at increasing coverage depths (a measure of coverage quality and uniformity). For variant detection (Table 9), a bioinformatic filter was applied to generate an in silico representation of the 83 hearing loss genes included in the NBDxv1.0 panel. Additionally, specimen types from the same individuals were used such that SNPs could be directly compared in a pair-wise fashion, Minimal filtering was used in order to generate sufficient representation while maximizing calling quality (e.g., SNP calls are not from low frequency sequencing errors; Hodges et al. 2009). This can be used as a proxy to approximate the ability to find variants from each sample type, and thus identify any systemic issues that could lead to a missed identification of causal variants based on the specimen source. For six in silico sets, filtering resulted in ˜80-100 SNPs per sample pair and demonstrated consistency in reads from the sample types. The largest difference from Exome sequencing was 3 variants. The unmatched variants were found to be in areas of lower coverage (e.g., 6-25 total reads on the specific SNP in a sample with median coverage >70×) or reads of the alternate allele close to a filtering threshold (e.g., frequency 0.32), suggesting the difference in some cases was not specifically due to any particular specimen source. Larger differences were observed for the same sample with Exome capture as compared to capture with an NBDx panel (described below), with an increase in variants where NBDx has full gene coverage.

TABLE 9
Concordance from biospecimens compared
with in silico Hearing Loss gene panel.

| Sample | Sample | in silico | Unique to | Unique to |
|---|---|---|---|---|
| Type 1 | Type 2 | SNP Count* | Type 1 | Type 2 |
| DBS | Whole Blood | 103 | 1 | 3 |
| DBS | Whole Blood | 90 | 0 | 2 |
| DBS | Saliva | 82 | 1 | 0 |
| DBS | Saliva | 79 | 0 | 0 |
| DBS | DBS + WGA | 83 | 3 | 0 |
| DBS | DBS + WGA | 79 | 0 | 0 |
| DBS (PGDx) | DBS + WGA | 92 | 1 | 1 |
| | (PGDx) | | | |
| DBS | DBS (PGDx) | 91 | 1 | 7 |

*SNP count, of Hearing Loss genes on the PGDx v1.0 panel with Coverage >5 reads, Protein impact and Heterozygous allele at >30% of reads.

The initial DNA characterization can raise concerns due to bacterial contamination in DNA isolations from saliva. Two factors can eliminate detection of these sequences in the final reads—hybrid capture and mapping sequencing reads to the human reference. However, the ultimate consequence for saliva samples could be lower target coverage and a resulting reduction in accurate variant calls. Such issues were not apparent from the analysis. Among the paired in silico comparisons of saliva samples (with up to 20% bacterial contamination), target coverage was similar to blood and DBS (see FIG. 4B) as was variant calling (Table 9). This can be due to de-enrichment of bacterial contamination in TNGS, but in some cases would be a concern in whole genome sequencing.

TNGS Using DNA Isolated from DBS and Amplified by Whole Genome Amplification (WGA) Method

WGA as an option was explored because of low yield of DNA isolations from bio-specimens such as DBS. As noted above, DNA yields from DBS spots were generally more than sufficient for NGS library preparation (e.g., current library construction protocols use as little as 50 ng DNA input). However, protocols can be used for any low yield patient samples. WGA could also expand sample prep options for simpler and faster workflows in the future. Previous findings have suggested WGA of DBS isolated DNA in some cases could be successfully used for sequencing-based variant calls (Winkel et al. 2011, Hollegaard et al. 2013). As such, WGA from DBS isolated DNA was tested using the RepliG UltraFast kit (Qiagen). RepliG UltraFast utilizes phi29 based Multiple Displacement Amplification (MDA) technology to produce amplified material in 1.5 hours, as compared to overnight for the standard kits, and a minimal input of 10 ng DNA. For three DBS samples, WGA input totals of 20-30 ng gave post-amplification yields of 3-4.5 ug, representing a 100-230× amplification. A lowest DNA yield from a clinically-derived blood spot without WGA has been ˜100 ng. WGA can provide the ability to increase low yield sample to an amount sufficient for several NGS libraries as well as potential archiving.

In some cases, a concern with WGA is loss of specific regions or mutations due to biases in amplification. This can be of particular interest for cancer samples, where tumor markers are not present in all genomic copies (rare variants). However, there can also be somatic variants with high representation. For a WGA performance test, two of the DBS samples were taken and performed Exome sequencing from the same DNA pre- or post-WGA, as described above. For closest comparison, these were run side-by-side multiplexed within the same sequencing lane on the Illumina HiSeq2500. Results are summarized in FIG. 4B and Table 9, The WGA did have some consequences for % of reads mapping (˜10% lower), target read coverage (5-10% lower) and overall quality of reads (% SNV with quality >40 dropped from a high quality 96% to moderate quality of 93%). However, variant detection remained robust in two filtering regimens. First, WGA samples were compared by standard filtering for causal variant detection (Coverage >5, Protein Impact, Minor Allele Frequency (MAF)>5%, and classified as probably damaging). All variants found in the pre-WGA samples were also identified post-WGA. Further comparison with the hearing loss in silico filters described above found few differences in SNP calls, One sample had 3 SNPs in the pre-WGA sample only, all of which were in low coverage regions. Consistent with the lower on-target reads for WGA samples, increasing the minimal coverage threshold reduces the correlation. Similar results were obtained for samples run with and without WGA on an NBDx panel. In combination with the additional cost and workflow time, these results suggest WGA should not be a first choice of use. However, in cases of low yield that can require additional sample (or otherwise no results), WGA provides an option to obtain rapid preliminary results so physicians and families can start to consider appropriate action while awaiting confirmatory results (such as cessation of aminoglycoside administration in cases of mitochondrial mutations).

Handler and Cross-Contamination Assay

A potential source of contamination, beyond the bacterial contamination discussed above, can be from sample handling, both from the sample handler and cross-sample. Such contamination can be a potential concern for miscalls on variant status and diagnosis. In order to examine this aspect, an approach was arranged to look specifically for the presence of sample handler variants being detected in other isolated samples. Samples were prepared in parallel from the handler's own specimen as well as an unrelated individual. These were then subsequently taken in parallel through library prep, hybrid capture and Exome sequencing at YCGA. An independent sample from the non-handler was also isolated and Exome sequenced at another facility (Covance). This allowed distinction between contamination and variants truly shared between the handler and non-handler. Additionally, samples sequenced at YCGA prior to these, using DNA which again was not isolated at our facility, were used to control for variants commonly identified in samples processed at that facility. As outlined below, analysis of almost 600 variants did not find misidentification of non-handler mutations due to contamination from the handler's DNA. Following Exome sequencing 25,149 variants were identified in the handler specimen. A first pass filter was applied to remove many of the common variants within the general population (MAF 1-5%), sufficient coverage to avoid false representation within this sample (≧20 reads) and to identify variants with protein impact. This filter brought the number of variants in the handler down to 566 for further consideration. In the non-handler sample 24,955 total variants were identified and subjected to the same filtering. Of the 566 filtered variants in the handler sample, 49 were found to be in common with the non-handler sample. All 49 were also identified in the independent non-handler sample, indicating that the 49 variants are truly in common rather than due to contamination. Further, the ratios of alternate allele in heterozygotes were similar in the handler and nonhandler sample. Contaminating sequences would be expected to represent a lower fraction of reads in the contaminated sample. Bringing down the coverage level to ≧5 reads did not change the number of in common variants found. In total, none of the filtered variants identified in the handler sample were detected as contamination in the non-handler sample.

Preliminary Studies on CMV Enrichment and Detection

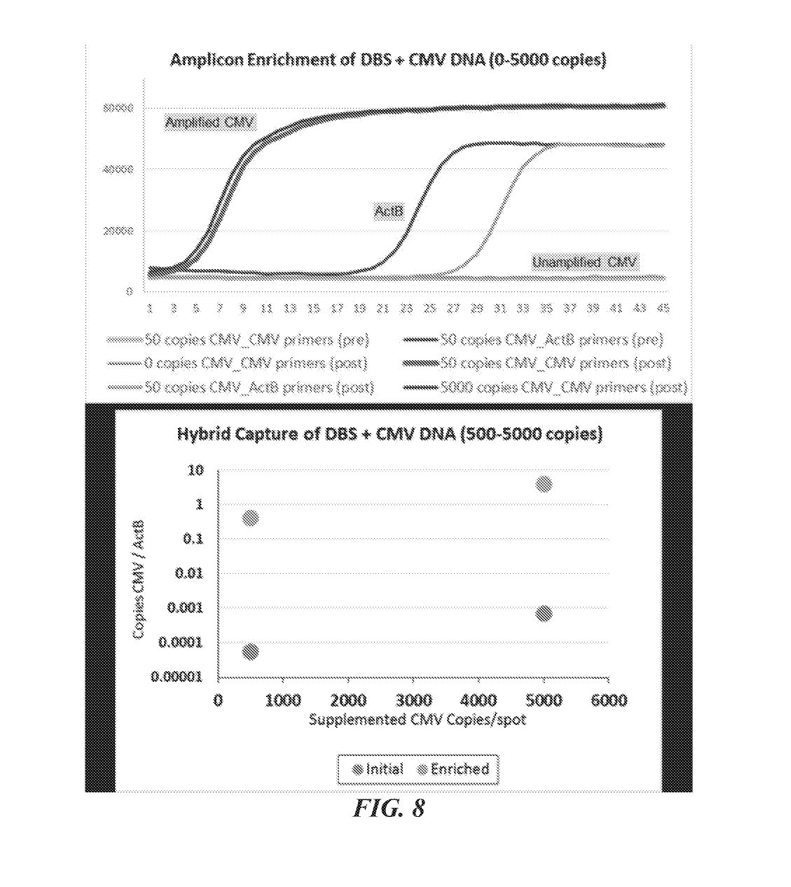

A truly comprehensive hearing loss panel can include detection of both genetic causes as well as non-genetic such as CMV infection. Detection by sequencing of CMV directly from blood could suffer from low coverage (even with hybrid capture) as the endogenous human genomic sequences can be more highly represented. Cunningham et al. (2010) performed NGS from cultured fibroblast cells with 10,000-44,000 virus genomes/cell and were able to perform identification with CMV representing 3% of sequence reads. However, as the authors comment, the viral loads of patient infections would represent far less of the sequencing reads. In 2009 de Vries et al. estimated detection of CMV from DBS by qPCR and by their analysis CMV infection associated with heating loss could represent as little as 500 copies CMV per 50 ul whole blood that typifies a DBS. There are 1000-fold more human cells than CMVs per spot. By extending this to include the CMV genome which is ˜10,000-fold smaller than the human genome, it was estimated 10 million fold difference in the amount of bases of DNA from human compared to CMV. Although, 190,000-fold enrichment has been possible with hybrid capture methodology (Burbano et al., 2010) this is not readily achievable by hybrid capture in a single round of enrichment, To increase detection, a strategy was taken to specifically enrich CMV followed by recombining with the workflow for human genomic captured regions. As proof of concept for CMV enrichment, DBS isolated human DNA was spiked with DNA from CMV to represent a range of viral loads (0-5,000 copies/spot) and performed either amplicon-based target enrichment in combination with a human targeted panel (e.g., using the SmartChip TE™ system from WaferGen) or hybrid capture of CMV only. Both were performed in the presence of human genomic DNA, Enrichment was then assessed by qPCR using the copy number change of CMV sequences relative to an unenriched human sequence (ActB). Even with as little as 50 copies of CMV per DBS spot, amplicon-based enrichment showed 9-orders of magnitude enrichment of CMV from increased CMV, and concomitant decrease in ActB, for the same total DNA (FIG. 8). Additionally, the strong co-amplification of CMV did not impact that of the other human genes present on the panel. However, this method in some cases would be suited to sequencing for CMV strain identification as the standard conditions amplify to saturation, which can result in loss of quantitative detection. Alternatively, hybrid capture was performed using three biotinylated 60-mer oligos to enrich the same region of CMV that is detected in qPCR. Capture was performed using hybridization/wash buffers from Nimblegen and with a protocol analogous to a standard Exome and panel captures, with saturating levels of capture probe. The hybrid capture gave a 3000-fold enrichment of the viral sequence along with an approximate ratio of 10-fold between the 500- and 5000-CMV copies. Thus, hybrid capture better maintained the relative viral load of the samples. While not as sensitive as amplicon-based enrichment (50 copies/spot vs. 500 copies/spot detected), hybrid capture improve upon the baseline levels of qPGR alone (our pre-enrichment qPCR and de Vries et al. 2009). Altogether, the studies show that creating a comprehensive hearing loss assay with a targeted genetic panel and CMV detection could be accomplished through a combination of either qPCR CMV quantitation, SmartChip amplification for sequencing identification of CMV strain(s), or an offline hybrid capture of CMV to be integrated with target panel captured material.

Establish Hearing Loss Targeted Panel Newborns (NewbornDex™/NBDx v1.0)

Hearing Loss Test Panel Design

The NBDx v1.0 gene panel has 84 genes for non-syndromic and syndromic hearing loss. 46 of these targeted genes have full gene coverage in order to increase variant detection ability beyond the coding exons present in an Exome panel and most currently existing hearing loss targeted panels. The list of non-syndromic hearing loss genes was developed in part based upon literature review, with a major contribution from OMIM as well as from two comprehensive publications (Resendes et al. 2001, Duman and Tekin 2013). In addition, a number of syndromic hearing loss genes were added to the list of non-syndromic genes. These genes were selected because the overt clinical symptoms, in some cases, cannot be obvious in the neonatal period. The panel also includes 126 Newborn Screening genes, 10 with full gene coverage, and 90 genes for hepatomegaly, hypotonia and failure to thrive. The additional coverage can play a role in determination of syndromic hearing loss potential. In total, the panel covers a 7 Mb target of highest probability for detection of hearing loss causal variants while maximizing coverage with a given sequencing capacity. The final tiled probe set covers 92% of the targeted bases. A comparison of hearing loss gene coverage in the NBDx panel was compared to other commercially available tests. As examples, the tiled probe design of NBDx v1.0 covers the full genes of GJB2 and GJB6. For comparison, the Inherited Disease panel from Illumina, one option for a newborn genetic panel, covers only 10 hearing loss genes, with exonsonly of GJB2 and does not include GJB6 or mitochondrial regions (MTRNR1).

Exome and Panel Analysis of Hearing Loss Samples

Panel Sequencing

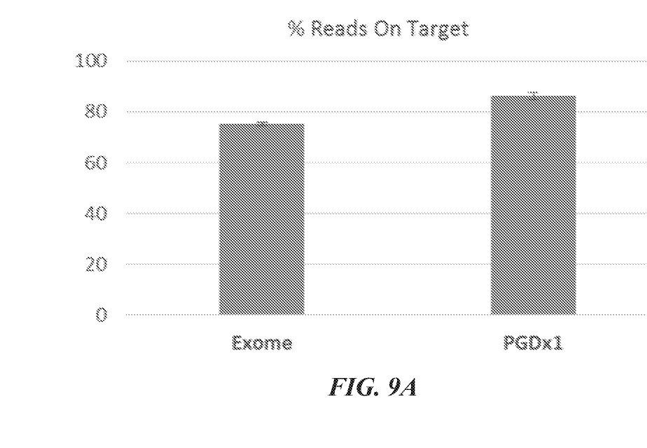

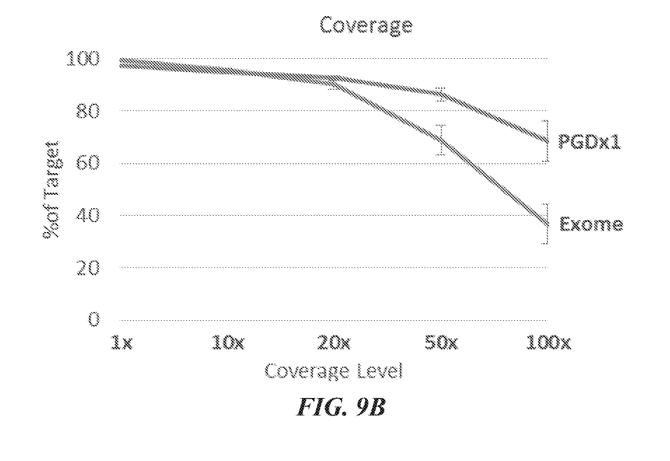

The NBDx v1.0 panel was used for multiplexed hybrid capture with 20 samples per single capture, and one sequencing lane of the Illumina HiSeq 2000/2500. Samples included those with known hearing loss mutations as well as various control samples from whole blood, DBS and archival DBS stored at room temperature for ˜10 years. By comparison, Exomes are handled as 4 samples/run. For direct performance comparison, 4 of the hearing loss samples were run with both the Exome and NBDx v1.0 capture. The NBDx v1.0 panel has percent reads on target similar or greater than the Exome (FIG. 9A). Additionally, with 5× more samples/run for NBDx v1.0 as compared to the Exome, smaller target allows for greater coverage depth with more samples run in unison (FIG. 9B). Three sets of NBDx v1.0 capturing and sequencing have been performed. Due to IRB restrictions a per-sample identification is not reported, but rather present here a summation of the mutations found. In total, 67 representative mutations in non-syndromic and syndromic hearing loss genes including missense, indels and splice site are listed here (Table 10).

TABLE 10
Representative hearing loss mutations identified by TNGS
with the NBDx v1.0 panel.

| | Trascript | Protein | # | |
|---|---|---|---|---|
| Gene | Variant | Variant | cases | Effect |
| BTD | c.1459T>C | p.Trp187Arg | 1 | Non-synonymous |
| BTD | c.1330G>C | p.Asp444His | 1 | Non-synonymous |
| BSND | c.3G>A | p.Met1Ile | 1 | Non-synonymous |
| CDH23 | c.1117G>A | p.Ala373Thr | 1 | Non-synonymous |
| CDH23 | c.3308A>G | p.Asn1103Ser | 1 | Non-synonymous |
| CDH23 | c.3632C>T | p.Pro1211Leu | 1 | Non-synonymous |
| CCDC50 | c.363A>T | p.Leu121Phe | 1 | Non-synonymous |
| CCDC50 | c.868C>T | p.Arg290Trp | 1 | Non-synonymous |
| DFNB59 | c.86A>G | p.Asp29Gly | 1 | Non-synonymous |
| DSPP | c.1298G>T | p.Gly433Val | 1 | Non-synonymous |
| EYA1 | c.58C>G | p.Pro20Ala | 1 | Non-synonymous |
| FLT4 | c.2860C>T | p.Pro954Ser | 1 | Non-synonymous |
| GJB2 | c.35_35delG | p.Ser12del | 11 | Frameshift Deletion |
| GJB2 | c.79G>A | p.Val27Ile | 3 | Non-synonymous |
| GJB2 | c.130T>C | p.trp44Arg | 1 | Non-synonymous |
| GJB2 | c.167_167delT | p.Ser56del | 1 | Frameshift Deletion |
| GJB2 | c.235_235delC | p.Ser79del | 1 | Frameshift Deletion |
| GJB2 | c.269T>C | p.Leu90Pro | 1 | Non-synonymous |
| GJB3 | c.219C>A | p.Asn73Lys | 1 | Non-synonymous |
| GJB3 | c.316C>T | p.Arg106Cys | 1 | Non-synonymous |
| GRHL2 | c.26A>G | p.Lys9Arg | 1 | Non-synonymous |
| GRHL2 | c.1243G>A | p.Val415Ile | 1 | Non-synonymous |
| ILDR1 | c.1193G>A | p.Arg398His | 1 | Non-synonymous |
| KCNJ10 | c.811C>17 | p.Arg271Cys | 5 | Frameshift Deletion |
| KCNQ4 | c.1427C>T | p.Pro476Leu | 1 | Non-synonymous |
| MARVELD2 | c.482C>T | p.Ser161Phe | 1 | Non-synonymous |
| MCOLN3 | c.535C>G | p.Pro179Ala | 1 | Non-synonymous |
| MCOLN3 | c.1223C>T | p.Ala480Val | 1 | Non-synonymous |
| MYH14 | c.1150G>T | p.Gly384Cys | 1 | Non-synonymous |
| MYO1A | c.454C>T | p.Arg152Cys | 1 | Non-synonymous |
| MYO1A | c.2710C>T | p.Arg904Cys | 1 | Non-synonymous |
| MYO1A | c.2021G>A | p.Gly674Asp | 1 | Non-synonymous |
| MYO1A | c.2390C>T | p.Ser797Phe | 1 | Non-synonymous |
| MYO3A | c.2497G>T | p.ala833Ser | 3 | Non-synonymous |
| MYO6 | c.3215T>C | p.Ile1072thr | 1 | Non-synonymous |
| MYO7A | c.905G>A | p.Arg302His | 1 | Non-synonymous |
| MYO15A | c.2225G>T | p.Arg742Leu | 1 | Non-synonymous |
| NKX2-5 | c.73C>T | p.Arg25Cys | 1 | Non-synonymous |
| OTOF | c.3629G>A | p.Arg1210Gln | 1 | Non-synonymous |
| OTOF | c.2273G>A | p.Arg758His | 1 | Non-synonymous |
| OTOF | c.1350C>G | p.Asp450Glu | 1 | Non-synonymous |
| PCDH15 | c.1781G>T | p.Arg594Leu | 1 | Non-synonymous |
| PDZD7 | c.572T>A | p.Val191Glu | 1 | Non-synonymous |
| PRPS1 | c.526C>T | p.Pro176Ser | 1 | Non-synonymous |
| SIX5 | c.1655C>T | p.thr552Met | 1 | Non-synonymous |
| STRC | c.4466A>C | p.Glu1489Ala | 1 | Non-synonymous |
| TECTA | c.5012C>T | p.Ser1671Leu | 1 | Non-synonymous |
| TJP2 | c.143T<>C | p.Val48Ala | 1 | Non-synonymous |
| TJP2 | c.2004G>A | p.Met668Ile | 6 | Non-synonymous |
| TJP2 | c.2128G>T | p.Val710Leu | 1 | Non-synonymous |
| TJP2 | c.3029C>T | p.Ser1010Phe | 2 | Non-synonymous |
| TMC1 | c.421C>T | p.Arg141trp | 1 | Non-synonymous |
| TMPRSS3 | c.1042G>A | p.Asp348Asn | 1 | Non-synonymous |
| TRIOBP | c.1979C>T | p.Ala660Val | 5 | Non-synonymous |
| TRIOBP | c.2450C>G | p.Thr817Ser | 3 | Non-synonymous |
| USH1C | c.496 + 1G>T | | 1 | Splice Site |
| USH2A | c.2137G>C | p.Gly713Arg | 1 | Non-synonymous |
| USH2A | c.10246T>G | p.Cys3416Gly | 3 | Non-synonymous |
| USH2A | c.13763C>A | p.Ser4588Tyr | 1 | Non-synonymous |
| USH2A | c.14074G>A | p.Gly4692Arg | 1 | Non-synonymous |
| WFS1 | c.353A>C | p.Asp118Ala | 1 | Non-synonymous |
| WFS1 | c.683G>A | p.Arg228His | 1 | Non-synonymous |
| WFS1 | c.1277G>A | p.Cys426Tyr | 1 | Non-synonymous |
| WFS1 | c.1597C>T | p.Pro533Ser | 1 | Non-synonymous |
| WFS1 | c.2051C>T | p.Ala684Val | 1 | Non-synonymous |
| WFS1 | c.2053C>T | p.Arg685Cys | 1 | Non-synonymous |

### Example 3

### Newborn Screening for Inherited Metabolic Disorders and Rare Genetic Syndromes Using NGS

Patients and Methods: The specimens were collected under informed consent as part of diagnostic and research protocols approved by the Lancaster General Hospital, PA and the Western Institutional Review Board, WA. DNA and biospecimens to validate a methodology was obtained from patients with known mutations in the Amish/Mennonite population in collaboration with the Clinic for Special Children (CSC) in Strasburg, Pa. The disease causing mutations were initially characterized by traditional Sanger DNA sequencing at CSC.

Patient DNA was enriched by hybrid-capture [Roche Nimblegen SeqCap EZ Human Exome Library v2.0 or SeqCap EZ Choice for the targeted panel], sequenced on the Illumina Hi-Seq 2000/2500 and analyzed using a custom analysis pipeline (see FIGS. 1A and 1B and General Methodology below for overview). Following sequencing, FASTQ files were generated for each sample and processed through an automated bioinformatic decision tree developed with two bioinformatics partners (Curoverse and Omicia) to generate variant files and to identify and categorize genotypic variations. Curoverse processed FASTQ files on the Arvados platform (arvados.org) for reference genome alignment (hg19/GRCH build 37) and variant calling using the BWA aligner (Li and Durbin, 2009) and GATK2 toolkit (McKenna et al. 2010; DePristo et al. 2011), Variant files were uploaded into a comprehensive genome interpretation software, Opal (Omicia, Emeryville, Calif.), to identify disease causing variants. Opal pre-classifies each variant in pathogenicity classes such as pathogenic, likely to be pathogenic, or benign such as suggested and published by the American College of Medical Genetics. In silico filters available within Opal were used for gene set selection and database comparison (Clinvar, OMIM, LSDB). Valiant pathogenicity was determined by either previous knowledge in the databases or molecular impact prediction algorithms (FIG. 7). The genes with mutations that had protein impact and low frequency (<5% in the general population) were readily identified. Parallel processing of 8-10 Exomes per 105 hours was demonstrated, and can be several hundred per week on a TNGS panel (APHL Meeting, Atlanta 2013). With one amplicon method, even shorter turnaround times can be achieved and therefore higher throughput per week. However, in some cases there are limits on up to 5000 amplicons of 700-bp each DNA input. These can be useful for designing complementary assays for gap-filling regions that are not targeted by hybrid-selection.

Establishment of the Genome-Scale Workflow and Sequencing Pipeline

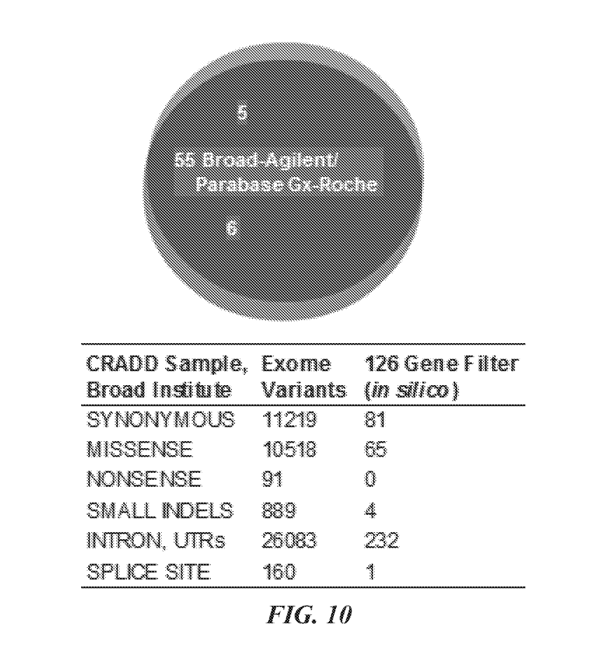

Approach for Experimental Design: In a clinical setting the incidental findings create an analysis and validation burden increasing time to answer and costs. This can be mitigated by application of an in silico gene filter to allow for automated variant analysis from larger sequencing sets, such as WES and gene panels containing >100 genes (e.g. NewbornDX™). An in silico gene filter that only calls variants in 126 genes relating to diseases either mandated by NBS programs, conditions that can be used to monitor in the newborn period was developed. The 126 NBS gene in silky filter was applied to Exome sequencing data on Amish/Mennonite patient samples obtained from the Clinic for Special Children (CSC), Strasbourg, Pa. The gene filter can be customized to include additional genes, such as for common symptoms seen in infants under care in the NICU or metabolic clinics difficult to distinguish with just symptomatology. The in silico panel data demonstrated at least two orders of magnitude reduction in incidental variants (Table 4 and FIG. 10), and therefore suggest the ease of making variant calls in targeted panels based on disease genes, clinical symptoms, or disease organs as filters. This allowed us to compare performance of samples across both Exome panel and the NBDx v1.0 targeted panel and also to compare the methods against low blood volumes or DBS samples. The full sample to interpretation can be accomplished for 8 parallel Exomes and minimum of 40 targeted panel samples in 105 hours.

Methodology for Hybrid Selection (DNA Capture)

Briefly, the following steps are involved for NGS: a) collection of various biological specimens such as dried blood spots, saliva, or whole blood, b) Genomic DNA isolation and, optionally, DNA amplification using whole genome DNA amplification strategies. Following DNA isolation (described later in Milestone c), the sample DNA can be fragmented and adapted into an NGS library by attachment of short sequences for sample identification and sequencing priming. The NGS library is denatured and incubated with a pool of tens of thousands of oligonucleotide probes for enrichment of DNA regions. NGS of the captured targets was performed with Illumina HiSeq 2000/2500 at YCGA and sequence aligned with Parabase Genomics collaborators at Curoverse.

Establishment and Evaluation of a LifeTime RAREDX™ in Silico Gene Filtering Panel

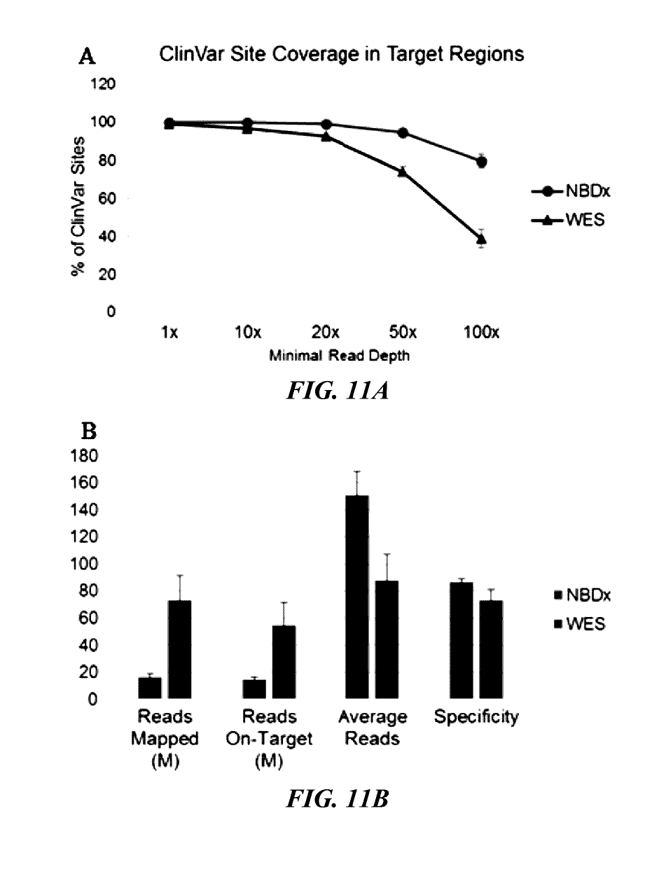

Propionic academia (PA) and Maple Syrup Urine Disease (MSUD), two metabolic diseases, which are routinely tested as part of newborn screening programs, are ideal for initial workflow validation of detection by targeted sequencing panels including Exome/LifeTime RAREDX™, and subsequently automated. From two independent sample types (blood and DNA) the same set of variants were recovered through a pipeline (Table 11), including nonphenotype related heterozygous pathogenic variants (carrier statuses), and demonstrated high concordance with results from the Broad Institute's Exome sequencing and variant calling (FIG. 10). After calibration with the two validation samples, 8 retrospective samples were processed and selected randomly from a validation set of 120 samples from CSC, on a Hi-Seq 2500 pipeline in rapid run mode which runs in 24 hours. The entire workflow from blood sample isolation through target-capture, sequencing on a HiSeq 2500 in rapid run mode, informatics and interpretation was parallel processed within 105 hours (FIG. 1B). These 8 samples were processed and interpreted in a blinded fashion as to the disorder and previous Sanger sequencing mutation identification, and results are summarized in Table 4. The data through a set of variant filters were analyzed and narrowed down. The filtering conditions used included: protein impacting, variant minor allele frequency (MAF) of <5%, evidence of pathogenicity, etc. Additionally two in silico gene filters were used, one reflecting 552 hereditary disorder gene panel (Saunders et al. 2012 and Illumina) and a 126 NBS gene filter. Using the 126 gene in silico filter the correct disorder and mutation, as previously validated by Sanger sequencing, was quickly identified by TNGS in all 8 samples. The IL7R and MTHFR mutations were not detected by the 552 in silico gene filter as they are not included in that panel. One patient with PKU was suspected to be a compound heterozygote for PAH [(782 G>A/284-286delTCA) OMIM#261600]. This patient also had a heterozygous mutation in MCCC2 (OMIM#609014) common in the Amish population. A similar situation was found in the patient with 11-β-Hydroxylase Deficiency as the patient was found to be a carrier for the 646 G>A mutation responsible for Adenosine Deaminase Deficiency. This mutation is also known to segregate in the Amish population. All other samples were found to be homozygous for the common mutations known to occur in these populations (Table 4). The bioinformatic analysis on Opal 3.0 made use of annotation optimization on two calls. The performance of the data indicated that on average 87% of the target was covered at 20× or more, and 73% of the captured sequencing reads were in WES target regions (FIGS. 11A and 11B).

TABLE 11
Exome sequencing from sample types (DNA, whole blood, DBS)

| | | | | PI + PD | | | | | |
|---|---|---|---|---|---|---|---|---|---|
| | | | | Hom. | | | | | |
| | | | Protein | Reads | | | | | |
| | | | impact | >5 | | | | | |
| Sample | | | (PI) | MAF | | | Transcript | Protein | |
| Type | ID | Disease | Variants | <5% | Gene | Reads | Variant | Variant | Zygosity |
| DNA | 28480 | Maple Syrup | 10,217 | 19 | BCKDHA | 35 | c.1312 T>A | p.Tyr438Asn | Hom. |
| | | Urine Disease | | | | | | | |
| DNA | 17235 | Mental | 10,329 | 21 | CRADO | 15 | c.382G>C | p.Gly128Arg | Hom. |
| | | Retardation | | | | | | | |
| DNA | 28839 | Propionic | 10,451 | 15 | PCCB | 5 | c.1606A>G | p.Asn536Asp | Hom. |
| | | Acidemia | | | | | | | |
| Whole | 28839 | Propionic | 14350 | 11 | PCCB | 18 | c.1606A>G | p.Asn536Asp | Hom. |
| Blood | | Acidemia | | | | | | | |
| DBS | 28839 | Propionic | 10,635 | 11 | PCCB | 49 | c.1606A>G | p. Asn536Asp | Hom. |
| | | Acidemia | | | | | | | |
| Whole | DA | Hyperglycinuria | 12,039 | 16 | SLC6A20 | 70 | c.596C>T | p.Thr199Met | Hom. |
| Blood | | | | | | | | | |
| DBS | DA | Hyperglycinuria | 12,056 | 13 | SLC6A20 | 88 | c.596C>t | p.Thr199Met | Hom. |

Case Examples from the Clinic and Neonatal Intensive Care Unit

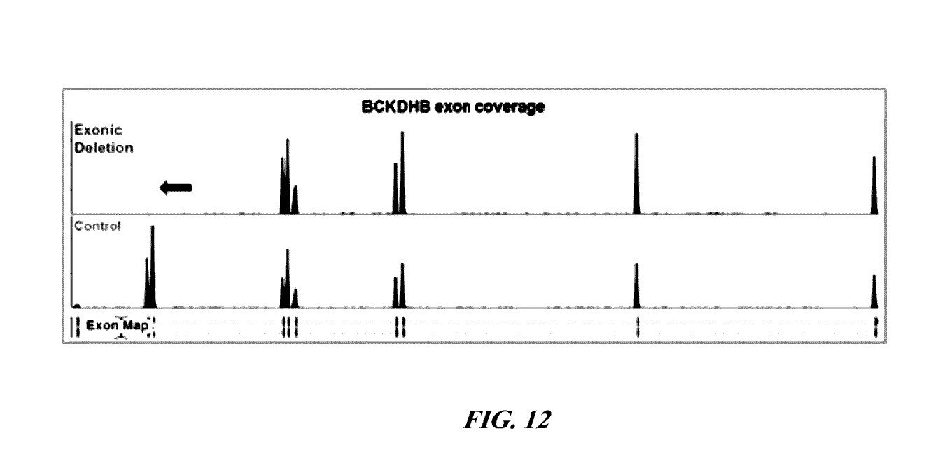

7 cases with Lifetime RAREDX™ highlighted the utility of an approach in the clinic. These include cases arriving at metabolic clinics and NICU, some already in crisis, spanning five disorders. Four cases had known or suspected deleterious mutations related to clinical phenotype quickly identified for Argininemia, Cystic Fibrosis (CF), Glutaric Aciduria (GA-1), and VLCAD deficiency. The specific mutations included non-synonymous, splice site, stop gained and deletion as either homozygous or compound heterozygous. Some of the mutations were novel and the known mutations were not canonical and would not have been detected by standard genotyping assays, Additionally, a suspected diagnosis of CF was confirmed as negative. In a case of Maple Syrup Urine Disease (MSUD) initial analysis gave no mutations in the four genes related to this disorder (BCKDHA, BCKDHB, DBT and DLD). Upon further examination coverage normalization against a control sample was able to be utilized to detect a large homozygous deletion spanning across exons 1-3 in BCKDHB (FIG. 12). In some cases, this type of analysis is not performed with WES and as such the mutation would have otherwise been missed. For both the CF and MSUD cases the NewbornDx™ panel would have added even more strength as the panel spans the entirety of both these genes.

The Lifetime RAREDX™ panel is also helpful in the newborn period as many Rare Genetic Diseases are not part of NBS or are encountered infrequently in the NICU and can require confirmation of clinical symptoms. A single test for Rare Disease Diagnosis is very useful in these cases. It was highlighted here a real world case of Multiple pterygium syndrome of the Escobar variant type (EV MPS; MIM:265000) from a NICU setting to demonstrate how post-natal testing subsequent to observations from prenatal ultrasound or initial examination can have clinical utility. According to the clinical information submitted by the hospital, a prenatal ultrasound noted multiple congenital abnormalities and amniocentesis showed a 46 XY karyotype with an apparently balanced chromosomal (8;16) translocation. Postnatal examination revealed clinical features consistent with the Escobar variant. Following Exome sequencing and focused interpretation the clinical report noted: heterozygous c.117dupC (p.N40fs) and c.401_402del (p.P134fs) mutations in the CHRNG gene. These were confirmed by Sanger sequencing of parental DNA that showed the two CHRNG gene variations to be in trans configuration (compound heterozygous) in this patient. Defects in CHRNG can be the cause of EV MPS, an autosomal recessive disorder characterized by excessive webbing (pterygia), congenital contractures (arthrogryposis), scoliosis, and variable other features. The finding of the compound heterozygous deleterious mutation in the CHRNG gene is consistent with the described clinical phenotype for this newborn.

DNA Isolation from DBS for Targeted Next-Generation Sequencing

The approach was to examine DNA isolation from newborn biospecimens for NGS including minimally and non-invasive sample collection sources such as DBS, saliva and small volume blood (25-50 ul). Utilization of these sample sources can allow us to better serve newborns by avoiding use of several milliliters of blood that is typical from NGS providers, a difficult request in some cases to meet for healthy babies and especially untenable for infants in the NICU setting. The DBS method can have particular advantages of being minimally-invasive and can be routinely performed. Also collection materials and techniques are standardized across US hospitals and other settings worldwide, and would be readily available from new patients as well as archives. NGS library preparation using enzymatic incorporation of barcoded universal adapters by ligation or transposition can make use of double stranded DNA (dSDNA), Several groups have isolated DNA from DBS for PCR based assays or amplicon sequencing, but these assays do not always require dsDNA (Lane and Noble 2010, Saavedra-Matiz et al. 2013). Isolation of dsDNA from DBS or 25 ul of whole blood for genome scale or TNGS has not been demonstrated or validated for clinical use, except for research protocols in methylation assays (Bevan 2012 and Aberg 2013). A robust reproducible clinical grade protocol that recovered sufficient dsDNA for TNGS library construction from DBS, 25 ul of whole blood and saliva, was developed. The dsDNA yield, and high MW DNA and purity, contaminating bacterial DNA, and enzymatic inhibition are shown in Table 8 and data performance in FIG. 4B. Subsequent isolations from a retrospective DBS set of over 20 separate patients gave variable yields (180 ng-4.4 ug, median of 435 ng). Such variability has been seen previously for both whole blood and DBS biospecimens (Suzanne Cordovado personal communication, Lane and Noble 2010, Abraham et al. 2012), yet in each case gave at least 150 ng which was sufficient for both QC and subsequent NGS analysis. Whole Genome Amplification (WGA) as a mitigation strategy was also tested for cases that fall below certain levels.

Performance of DNA Isolated from DBS with the LifeTime RAREDX™ Panel

The findings from DNA isolation trials with DBS demonstrated sufficient DNA yield, purity and quality to go forward into the targeted next-generation sequencing workflow. As an initial validation that DNA isolated from DBS did not produce subsequent biases in hybrid capture or NGS, two samples previously were run using the Lifetime RAREDX™ panel from whole blood again with DNA isolated from DBS. The results from DBS were indistinguishable from whole blood for the number of variants found and had high SNP concordance (Table 11), For each sample pair the same mutation was identified: PCCB c.1606 C>A (p.Asn536Asp) for sample 28839 and SLC6A20 c.596 C>T (p.Thr199Met) for sample DA. Further comparison of DNA from eight samples each of DBS and whole blood, plus two from saliva, were indistinguishable for the % reads on-target and the % target covered at various sequencing depths, indicating both a high rate and evenness of capture (FIG. 4B). Variants for these DBS samples also matched the expected calls. Similar comparisons with the NBDx panel further demonstrate matched performance between the biospecimen types.

Establishment of Newborn Specific Targeted Panel (NBDx) for the NewbornDx™ Test

NewbornDx™ Test Panel Design

The NBDx gene panel for TNGS was designed to selectively target genes relevant to diseases in the newborn period and includes the 126 NBS genes (described previously as in silico gene filter) whose exons are covered by capture probes across 1.4 Mb. Ten genes in this panel (CFTR, PAH, BCKDHA, BCKDHB, GCDH, PCCA, PCGB, BTD, CTNS and MTHFR) have intronic coverage to determine variations or deletion information similar to WGS for these genes. Additional genes related to common NICU symptomatology such as hepatomegaly, hypotonia and failure to thrive are included, with more conditions in the NICU under consideration. The performance of this panel from DBS and blood in initial tests has been compared against the Exome from 10 ml of blood and has shown equivalent results for the targeted regions.

Performance of the NewbornDx Test Panel (NBDx v1.0)

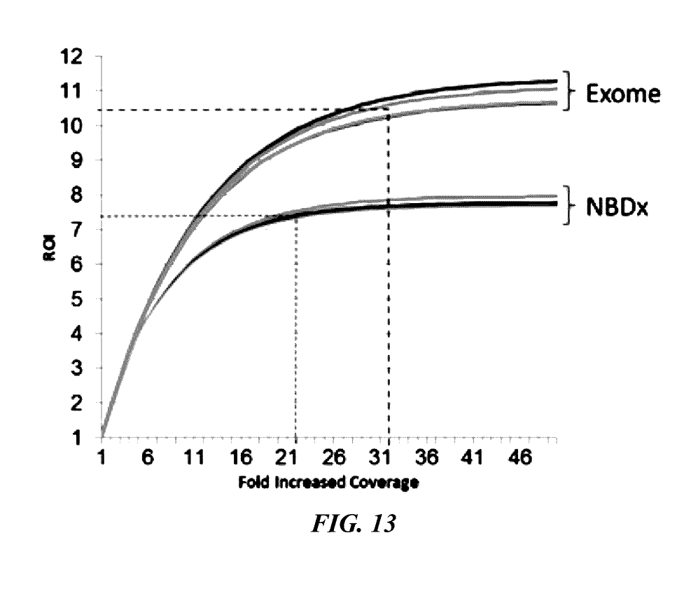

The NBDx panel was compared for hybrid capture performance against WES. NBDx captures were processed at 20 samples per lane of the Hi Seq2500 (rapid mode run), as compared to 4 samples for WES (FIGS. 11A and 11B). The average reads on-target was 2-fold higher for NBDx compared to WES (151× vs. 88×) due to focused sequencing combined with a higher on-target specificity relative to WES (87% vs 73%). The increased average sequencing depth in NBDx ensured fewer targeted regions would fall below stringent variant calling thresholds (Ajay et al. 2011, Meynert et al. 2013). This was demonstrated in coverage of ˜6215 ClinVar sites common to both WES and NBDx tiled regions, a measure that can be monitored for coverage in regions of clinical relevance in every sample (FIGS. 11A and 11B). At 10× coverage, NBDx achieved close to 99.8% coverage and at 1× 99.99% coverage (analytical sensitivity). However, while NBDx maintains 80% of the ClinVar sites covered at least 100×, WES significantly dropped to 39%. This result is also consistent with higher library complexity in WES compared to NBDx (FIG. 13).

Read depth can be a good predictor of variant sensitivity, and it was used to identify regions which are under-covered for the purpose of variant detection (FIGS. 5A and 5B). Sensitivity plots for CCM (FIG. 5A) and PAH (FIG. 5B) across chromosomal positions were generated for WES and NBDx as previously described by Meynert et al. 2013, Compared to NBDx, low sensitivity can be more likely in WES as there can be lower coverage due to GC bias or lack of probe coverage in intronic regions.

To assess uniformity, or relative abundance of different targeted regions, base distribution coverage was compared. Good uniformity was obtained on NBDx data sets, but WES skewed towards low coverage, likely reducing confidence on heterozygous calls (FIG. 6A). To assess reproducibility, pair-wise comparisons of coverage depth were performed at variant positions across independent sample preparation and sequencing. The analysis suggested that DBS, <1 ml whole blood and saliva provided a similar proportion of calls with a high agreement (Pearson Correlation Coefficient=0.9) between replicates (FIG. 6B).

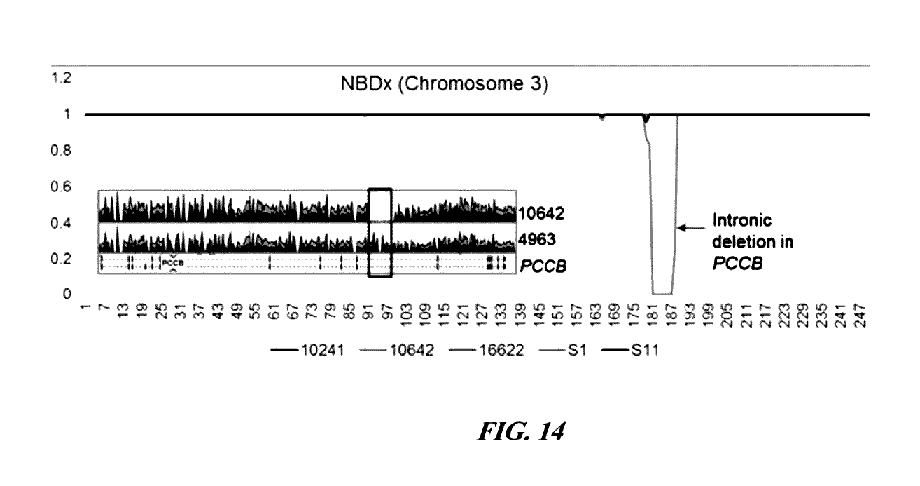

Another aspect of reproducibility measured is tiled region coverage between runs. The portion of the targeted region was sequenced with sufficient coverage to achieve 95% sensitivity for heterozygous calls (>13 reads). The maximum value per region was designated 1. An overlay of tiled regions in NBS genes on chromosome 3 is shown for 5 samples in FIG. 14. As unrelated samples are often run in sets of 4-20 in TNGS, highly variable regions such as homozygous deletions can be easily detected by comparing across samples. Using this concept and a simple statistic (Z-scoring) deletion spanning exons that correlated to phenotype was discovered. Additionally, in this dataset, completely embedded intronic deletions were detected in PCCB between Exons 10 & 11 (FIG. 14). Contiguous regions of genes can be covered by gene panels and makes them analogous to the whole genome sequencing approach. For example, FIG. 14 demonstrated that the main difference in PCCB analysis between a whole genome approach and gene panel approach of tiling or targeting entire gene is the method. For newborns, it may not be necessary to sequence every base of the human genome, but only focus on certain bases and/or genes for newborn disorders.

Capture and Sequencing Across Multiple Characterized Specimens Including GA-1

In collaboration with the Clinic for Special Children in Strasburg, Pa., specimens (DNA and blood spot) were obtained from Amish and Mennonite patients with different fully characterized mutations causing a variety of inborn errors of metabolism and genetic syndromes, including patients with PKU and GA-1. Performance of the NBDx gene panel was measured on 36 of the clinical samples from metabolic diseases (Table 4 and Table 6). These samples were also processed and interpreted in a blinded fashion as to the disorder and mutation present and were previously characterized by Sanger sequencing for causative mutations in 18 separate disease-related genes. Eight samples from this set were common with the WES analysis performed earlier and are described above. Eleven samples in the set had 19 different mutations spanning across the GCDH (glutaric acidemia Type I, GA-1) gene (arrows in FIG. 4A). As outlined in Table 4 above, the mutation(s) in each of the initial 8 cases were found using the LifeTime RAREDX™ and NBDx panels, including cases of heterozygous PAH mutations. By using the more targeted panel, the initial number of protein impact variants requiring consideration drops 40-fold from 14,000 for WES to 350 for NBDx (Table 4).

To assess the overall accuracy of the NGS genotype calls the a priori Sanger sequenced data was compared to call performance on NGS data. The variations ranged across a variety of mutation types including nonsynonymous variations, indels, stop gained and intronic/splice site variations (Table 1 and Table 6). 27 out of 36 cases were able to be predicted blindly after annotation correction (Sensitivity 75%; 95% CI: 57.79-87.85%), suggesting difficulty of calling in some cases without disease specific clinical phenotype, A reanalysis with clinical summaries confirmed an additional 7 cases, while 2 CYP21A2 cases were excluded (CSC ID 21901 and 27244) as the gene was omitted on the NBDx gene panel due to high homology with the CYP21A1 pseudogene. Thus, with clinical phenotype information correct calls were obtained on 32 out of 34 cases (Sensitivity of 94.12%; 95% CI: 80.29-99.11%). Separately, a second capture analysis was performed using the 552 gene hereditary panel (Illumina) that claims coverage of CYP21A2, and this approach failed to make the correct call likely due to misalignment or inability to distinguish reads from pseudogenes using TNGS. The two additional samples were carrier status-only (CSC ID 23275 and 30221).

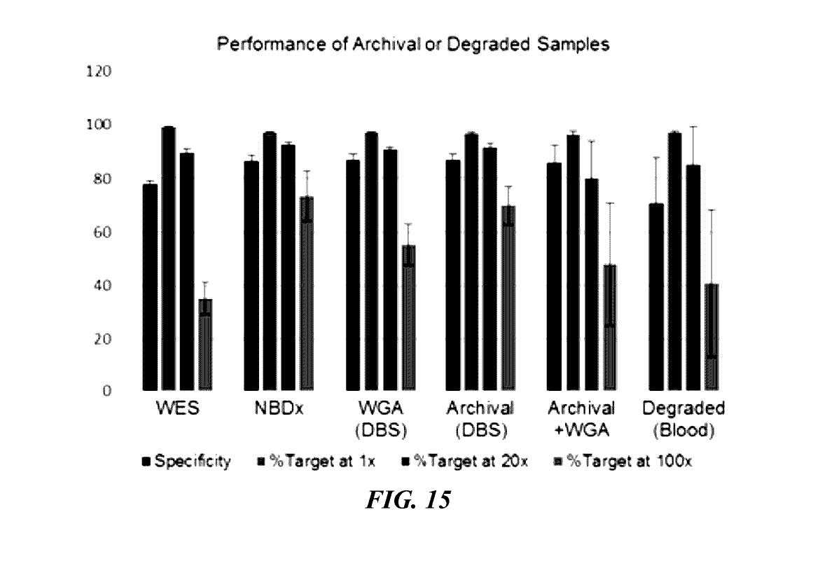

An additional 35 samples, including 17 mutations spanning across the PAH gene (Phenylketonuria, PKU), were run with NBDx panel as part of expanding a mutation database and further exploring technological capabilities. These included samples from 10 ml whole blood with moderate levels of degradation and archival DBS stored up to 10 years at room temperature. Varying levels of degradation (from moderate to severe) were seen in ˜30% of the samples from whole blood, either due to initial DNA isolation or sample storage. Similar variability in DNA isolated from DBS was not observed within a few months or stored frozen up to several years. However, the majority of archival DBS stored for several years at room temperature had lower DNA yields and varying levels of degradation. These samples were subjected to additional washes during DNA isolation and often subjected to subsequent WGA in preparation for NGS. Thus, these were not appropriate for direct comparison of capture performance between the NBDx and Exome panels, as can be seen from On-Target and Coverage metrics (FIG. 15). Despite the challenges, causative mutations could still be correctly identified from these samples, and as such they are useful for research-grade database development.

Comparison with Amplicon Enrichment

Performance of NBDx was also compared with an amplicon panel run on the WaferGen system, which utilizes a microfluidic chip to simultaneously perform up to 5000 individual PCR reactions. This approach was tested as a means to rapid target enrichment while avoiding biases and coverage variability of massively multiplexed PCR reactions. This technology worked, with the % On-Target and % Target covered up to 30× similar to hybrid capture panels. However, the DNA input to support the singleplex PCRs (350 ng per sample was used; 700 ng is recommended) can be 7-14× higher than other NGS library protocols (50-100 ng) and not consistent with typical DNA yields. Post-chip processing can involve subsequent NGS library production. This could be avoided through primer modifications, whole genome amplification of limiting DNA, but would limit amplicon size and decrease total target region coverage from the already smaller range of 1.5-2 Mb for a full chip.

Allele Dilution and Detection

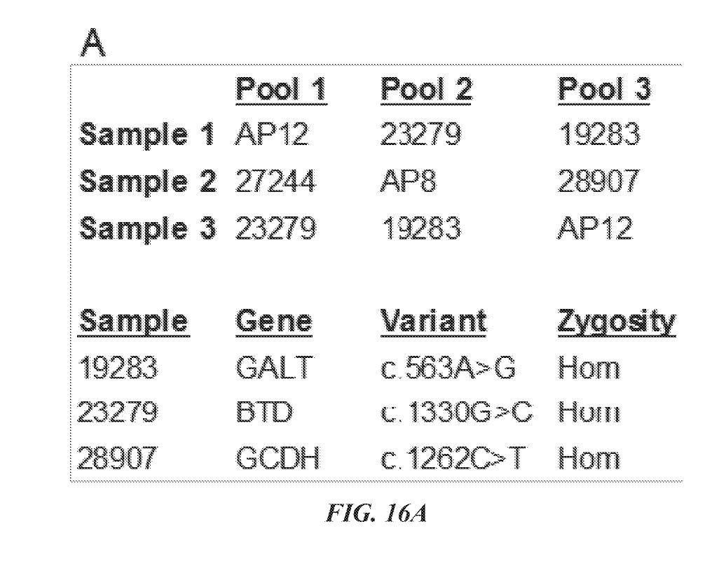

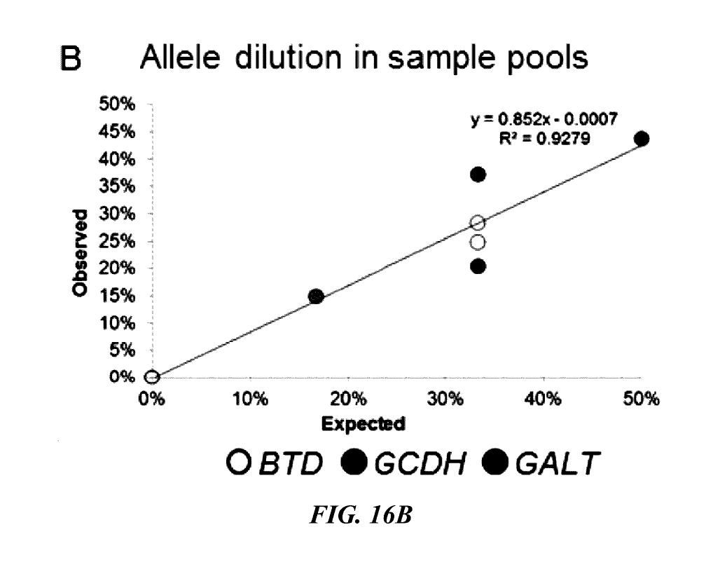

Rare homozygous variants (<5% MAF) at autosomal sites were followed to estimate analytical specificity, sequencing errors and DNA hybridization related allele bias. Six individual sample DNA were placed in three pools such that each pool had three unique patient samples and at least two was common to at least two other pools (FIG. 16A). As three of the Amish/Mennonite patients (CSC) had homozygous mutations in GCDH, GALT and BTD; the mixing experiment allowed us to observe expected vs. observed allele proportion for the homozygous variants across a range of pools and responded as expected (FIG. 16B). Lack of coverage or very low coverage in untargeted genes (e.g. HLA genes that were not covered or CYP21A2) demonstrated high degree of assay specificity when sequences were not targeted.

Pooling and Detection

Sample specific barcoding can involve independent processing and each cost $200-300. This $200 across 100 samples can be significant (i.e. $20,000). A pooled sample set can reduce cost if constructed in an ordered fashion and would be able to provide information on ultra-rare variants (e.g., at less than 1% MAFs in the population). DNA Sudoku strategy (Ehrlich 2009) can be used to reduce cost. Sudoku strategy works for sqrt N, where N is number of samples. So higher N can have a better cost advantage. For example, the pools can be in sets of 3 with overlaps and circular. In some cases, these samples are not barcoded individually but are at the pool level and have one member in common. However, the methods disclosed herein can avoid complexity and dependency on a large number of samples to start the process in Sudoku strategy. Unlike the open loop in Sudoku, the methods disclosed herein can use closed loops. If samples are mixed, cost can be reduced because unions of pool1,2, pool2,3 and pool3,1 can be used to pull out rare variants. Here 6 samples can be processed for the price of 3 as both barcoding and sequencing costs are reduced by half. The pools can be expanded to sets of four or more per pool.

Pooling Applications

A) Molecular autopsy: the methods disclosed herein can be used to find variants and/or cause during autopsy for coroner's office at lower cost; B) Screening technology: the methods disclosed herein can be used in supplemental and/or second tier newborn screening (NBS); C) Identifying drug target screening: the methods disclosed herein can be used to identify drug target. In some cases, identifying drug target screening may not be a definitive diagnosis at lower cost. D) Database building: the methods disclosed herein can be used in finding causal rare variants at lower cost and/or also separating the non-causal heterozygous rare mutations. E) Trio analysis: the methods disclosed herein can be used in analyzing de novo mutations for two trios, wherein each trio pool has one baby and two non-sanguinous or unrelated parents at lower cost or only has the parents in this mode to reduce cost by half (e.g., similar to NBS example); F) Specificity-dose response studies and signal predictions: In some cases, some homozygous calls at 200 read coverage can drop to heterozygous calls, but some may not change (common in population) or disappear (not very sensitive). G) Control Sequencing Errors: introducing contamination and sequencing errors can skew these ratios. In the absence of contamination or allele hybridization bias a clear dose response should be evident. A NSS internal control not seen in 1000 Genomes project (chimp or Neanderthal specific NSS variants) (Burbano et al 2010) can be spiked, for example in non-target portion of the genome, to see bias in real-time rather than offline measurements as can be done in NGS. Alternately, a well characterized control genome (e.g. NA12878 from HapMap) can be run along with test samples through library production and included with independent barcode indexes at a low percentage, such as 5-10%, in the multiplex capture library pool. Such pooling can allow direct measurement of contamination, sequencing errors and bias through the entire library and sequencing workflow without overwhelming sample throughput sequencing capacity. Sensitivity measurement: the methods disclosed herein can be used in measuring sensitivity because only ⅓ DNA is used and also because of the library complexity.

Pooling Experiment

The homozygous non-synonymous mutations in Amish/Mennonite can also be used to estimate contamination and/or capture sequencing errors or bias in autosomal sites using the fact that at every position the Amish/Mennonite individual was sequenced the genotype should be either homozygous common to Amish or Mennonite (monomorphic), homozygous to either Amish or Mennonite samples or a true variation. Six individual samples were mixed in three pools such that each pool had unique samples and at least two patients in each pool were common to at least two other pools. Therefore, without sequencing error or contamination, it was expected to see for each sample specific NSS variant in the pools either only homozygous calls (suggesting ancestral monomorphic allele), heterozygous calls in proportion to the dilution (if no interference), or an allele frequency of intermediate type due to interference of a similar common NSS variant. Measurements can also be independently made to monomorphic SNPs reported in the population. Additional alignment informatic control can be used for non-human genomes and variants.

### Example 4

### DNA Recovery Using Pressure Cycling

In the experiments additional benefits of pressure cycling on the activity of several enzymes were confirmed including proteinase K for DNA isolation, trypsin, Lys-C and chymotrypsin for proteomic analyses and PNGase F for protein deglycosylation. Namely, tissue or coagulated blood digestion by Proteinase K can be accelerated under pressure, resulting in faster isolation of intact unsheared genomic DNA. High pressure can alter protein conformation and hydrophobic interactions, acting on the compressible constituents of the sample resulting in destabilization of secondary structures, but not in the disruption of covalent bonds. Therefore, protein unfolding that occurs under high pressure can allow better access of proteases to the cellular proteins, but without the risk of damage to the DNA.

In experiments genomic DNA was extracted from duplicate rat liver and heart muscle samples with or without pressure-accelerated digestion. The pressure-treated tissues were subjected to pressure cycling consisting of 1 minute at 20,000 or 35,000 psi followed by 5 seconds at atmospheric pressure for 60-130 cycles. Control samples were digested for the same time and at the same temperature, but were held at atmospheric pressure (14.7 psi). When pressure cycling was performed at 20,000 psi at 55° C., complete lysis of rat heart muscle tissues was Observed after as few as 60 cycles, while visible pieces of undigested tissue remained in all control samples. Recovery of DNA was quantified using the QUBIT™ fluorimeter (Invitrogen). Results (Table 12) demonstrate that pressure cycling enhances Proteinase K activity, as indicated by both dissolution of tissue and by increased DNA recovery.

TABLE 12
PTC enhances proteinase K activity.

| | Time in | | | Avg recovery: μg | % of |
|---|---|---|---|---|---|
| Tissue | Minutes | Temp | Pressure | DNA per mg tissue | control* |
| Liver | 130 | Ambient | 35 kpsi | 0.66 (n = 2) | 228% |
| | | Ambient | Ambient | 0.29 (n = 2) | |
| | 90 | Ambient | 35 kpsi | 1.09 (n = 3) | 279% |
| | | Ambient | Ambient | 0.39 (n = 3) | |
| | 100 | Ambient | 20 kpsi | 1.47 (n = 5) | 155% |
| | | Ambient | Ambient | 0.95 (n = 3) | |
| Heart | 60 | Ambient | 20 kpsi | 0.60 (n = 2) | 155% |
| Muscle | | Ambient | Ambient | 0.39 (n = 2) | |
| | 120 | Ambient | 20 kpsi | 1.03 (n = 2) | 154% |
| | | Ambient | Ambient | 0.67 (n = 2) | |
| | 60 | Ambient | 20 kpsi | 3.95 (n = 3) | 153% |
| | | Ambient | Ambient | 2.59 (n = 3) | |

Pressure Cycling Technology (PCT) can be used to extract DNA from dried blood stains for forensic applications. Fresh whole human blood was used to prepare the bloodstained cloth, Samples were subjected to PCT in Tris-KCl buffer pH 8.0 for 5-10 cycles at 4° C. Control samples were incubated in the same buffer for 5 minutes at atmospheric pressure, DNA was amplified by PCR directly from the extracts without further purification or clean-up using primers specific for human mitochondrial DNA. The effect of pressure cycling on DNA yield from dried blood on cotton swabs (equivalent to 0.1 μl of blood per swab) was tested by comparing swabs that were pretreated with pressure for 1 hour, to controls that were treated without pressure. DNA was then extracted from the swabs using the Maxwell 16 platform, and quantified with the Plexor HY kit (Promnega). The pressure-pretreated samples exhibited an average 30% higher DNA yield compared to controls.

Other applications of pressure cycling can be used on enzymatic digestion for proteomic applications. PCT can accelerate trypsin digestion without sacrificing specificity. In addition, there is a detergent-free sample preparation technique from Pressure Biosciences, Inc. (PBI) which can allow for the concurrent isolation and fractionation of protein, nucleic acids and lipids from cells and tissues. This method can utilize a synergistic combination of cell disruption by PCT and a reagent system (ProteoSolve-SB kit) that dissolves and partitions distinct classes of molecules into separate fractions.

### Example 5

### Exemplary Gene Panels

The methods and systems disclosed herein can be used by sequencing the sample using gene panels or combination of gene panels. A few exemplary gene panels are listed herein.

TABLE 13
NBDxV1.1 Gene Panel

| | | | | | |
|---|---|---|---|---|---|
| AARS2 | AASS | ABAT | ABCA12 | ABCA3 | ABCC2 |
| ABCC8 | ABCD1 | ABCD4 | ACAD8 | ACAD9 | ACADL |
| ACADM | ACADS | ACADSB | ACADVL | ACAT1 | ACOX1 |
| ACSF3 | ACTA1 | ACTG1 | ADA | ADAMTS13 | ADK |
| AGA | AGL | AGXT | AHCY | AKR1D1 | AKT2 |
| ALAS2 | ALDH3A2 | ALDH5A1 | ALDH7A1 | ALDOA | ALDOB |
| ALK | ALMS1 | ALOX12B | ALOXE3 | ALPL | AMT |
| ANK1 | AP2S1 | APOC2 | AQP3 | ARG1 | ARL6 |
| ARSA | ARSB | ARX | ASIP | ASL | ASPA |
| ASPM | ASS1 | ATP2B2 | ATP6V1B1 | ATP7A | ATP7B |
| ATP8B1 | ATR | ATRX | AUH | BCKDHA | BCKDHB |
| BCS1L | BRAF | BRCA2 | BSND | BTD | C7orf10 |
| CA5A | CABP2 | CACNA1C | CACNA1D | CASK | CASR |
| CATSPER2 | CBS | CCDC50 | CCS | CD3D | CD3E |
| CDAN1 | CDH23 | CDK5RAP2 | CDKL5 | CDKN1C | CEACAM16 |
| CENPJ | CEP152 | CEP290 | CERS3 | CFTR | CHD7 |
| CHM | CIB2 | CLDN14 | CLPP | CLRN1 | COA5 |
| COCH | COL11A1 | COL11A2 | COL17A1 | COL1A1 | COL1A2 |
| COL2A1 | COL7A1 | COMP | COMT | COX10 | COX15 |
| CPS1 | CPT1A | CPT2 | CRTAP | CRYL1 | CRYM |
| CSTB | CTNS | CTRC | CTSD | CYP11B1 | CYP11B2 |
| CYP17A1 | CYP21A2 | CYP4F22 | D2HGDH | DBT | DCLRE1C |
| DDC | DECR1 | DEFB1 | DFNA5 | DFNB31 | DFNB59 |
| DHCR7 | DIABLO | DIAPH1 | DICER1 | DLAT | DLD |
| DMPK | DNA2 | DNAH11 | DNAH5 | DNAI1 | DNAJC19 |
| DSPP | DSTYK | DUOX2 | DUOXA2 | EDN3 | EDNRB |
| EGR2 | EIF2AK3 | ELANE | EPB42 | EPM2A | ESPN |
| ESRRB | ETFA | ETFB | ETFDH | ETHE1 | EVC |
| EVC2 | EYA1 | EYA4 | F10 | F11 | F13A1 |
| F2 | F5 | F8 | F9 | FAH | FANCA |
| FANCB | FANCC | FANCD2 | FBN1 | FBP1 | FGA |
| FGB | FGF3 | FGFR2 | FGFR3 | FGG | FKTN |
| FOXE1 | FOXG1 | FOXI1 | FOXRED1 | FRAS1 | FUCA1 |
| G6PD | GAA | GALC | GALE | GALK1 | GALNS |
| GALT | GAMT | GATA1 | GATA3 | GATM | GBA |
| GBE1 | GCDH | GCH1 | GCK | GCSH | GH1 |
| GIPC3 | GJB2 | GJB3 | GJB6 | GK | GLA |
| GLB1 | GLDC | GLUD1 | GLYCTK | GNA11 | GNAS |
| GNE | GNMT | GNPTAB | GP1BA | GP1BB | GP9 |
| GPC3 | GPHN | GPR98 | GPSM2 | GRHL2 | GRXCR1 |
| GSS | GUSB | GYS2 | H19 | HADH | HADHA |
| HADHB | HARS | HBA2 | HBB | HESX1 | HEXA |
| HEXB | HGD | HGF | HIBCH | HLCS | HMGCL |
| HMGCS2 | HNF1A | HNF1B | HNF4A | HPD | HPRT1 |
| HRAS | HSD17B10 | HSD17B4 | HSD3B2 | IDS | IDUA |
| IER3IP1 | IGF1 | IGF1R | IL2RA | IL2RG | IL7R |
| ILDR1 | INS | INSR | ITGA2B | ITGA6 | ITGB3 |
| ITGB4 | IVD | JAG1 | JAK3 | KCNE1 | KCNJ10 |
| KCNJ11 | KCNQ1 | KCNQ1OT1 | KCNQ2 | KCNQ3 | KCNQ4 |
| KDM6A | KLF1 | KMT2D | KRAS | KRT14 | KRT5 |
| LAMA2 | LAMA3 | LAMB3 | LAMC2 | LCK | LEPRE1 |
| LHFPL5 | LHX3 | LHX4 | LIAS | LIG4 | LIPA |
| LIPN | LMBRD1 | LOXHD1 | LRPPRC | LRTOMT | MAN2B1 |
| MAP2K1 | MARVELD2 | MAT1A | MCCC1 | MCCC2 | MCM4 |
| MCOLN3 | MCPH1 | MECP2 | MEF2C | MIR182 | MTR183 |
| MIR96 | MITF | MKKS | MLYCD | MMAA | MMAB |
| MMACHC | MMADHC | MOCS1 | MOCS2 | MPC1 | MPI |
| MPZ | MSRB3 | MT-RNR1 | MT-TS1 | MTHFR | MTR |
| MTRR | MUT | MVK | MYCN | MYH14 | MYH9 |
| MYO15A | MYO1A | MYO1C | MYO1F | MYO3A | MYO6 |
| MYO7A | NAA10 | NAGS | NDN | NDUFA11 | NDUFA2 |
| NDUFA9 | NDUFAF5 | NDUFS2 | NDUFS4 | NDUFV2 | NEU1 |
| NF1 | NFU1 | NHEJ1 | NHLRC1 | NIPAL4 | NIPBL |
| NKX2-1 | NKX2-5 | NOTCH2 | NPC1 | NPC2 | NR0B1 |
| NRAS | NSD1 | OAT | OGDH | OPA3 | OPLAH |
| OPRM1 | OTC | OTOA | OTOF | OTOG | OTOGL |
| OXCT1 | PAH | PAX2 | PAX3 | PAX8 | PC |
| PCBD1 | PCCA | PCCB | PCDH15 | PCK1 | PCNT |
| PDHA1 | PDHB | PDHX | PDP1 | PDX1 | PDZD7 |
| PEPD | PET100 | PHGDH | PHOX2B | PKD1 | PKD2 |
| PKHD1 | PKLR | PLEC | PLOD1 | PMM2 | PMP22 |
| PNP | PNPLA1 | PNPO | PNPT1 | POLG | POMC |
| POMT1 | POMT2 | POU1F1 | POU3F4 | POU4F3 | PPM1K |
| PRC1 | PRKAG2 | PRODH | PROP1 | PROS1 | PRPS1 |
| PRSS1 | PSAP | PSAT1 | PSEN1 | PSPH | PTF1A |
| PTPN11 | PTPRC | PTPRQ | PTS | QDPR | RAB18 |
| RAB3GAP1 | RAB3GAP2 | RAF1 | RAG1 | RAG2 | RB1 |
| RBBP8 | RBM8A | RDX | RET | RMRP | RPS19 |
| RPS6KA3 | SALL1 | SALL4 | SBDS | SCN1A | SDHA |
| SDHAF1 | SERAC1 | SERPINA1 | SERPINB6 | SERPINC1 | SERPING1 |
| SFTPB | SFTPC | SFTPD | SHOC2 | SIX1 | SIX5 |
| SLC16A1 | SLC17A3 | SLC17A8 | SLC22A5 | SLC25A1 | SLC25A13 |
| SLC25A15 | SLC25A19 | SLC25A20 | SLC26A2 | SLC26A4 | SLC26A5 |
| SLC2A1 | SLC37A4 | SLC46A1 | SLC4A1 | SLC52A1 | SLC5A5 |
| SLC6A1 | SLC6A8 | SLC7A7 | SLC9A6 | SLCO1B1 | SLCO1B3 |
| SMN1 | SMN2 | SMPD1 | SMPX | SNAI2 | SNRPN |
| SOS1 | SOX10 | SOX2 | SOX3 | SOX9 | SPINK1 |
| SPR | SPRED1 | SPTA1 | SPTAN1 | SPTB | ST3GAL5 |
| STAR | STIL | STRC | STXBP1 | SUCLA2 | SUCLG1 |
| SUMF1 | SUOX | TAT | TAZ | TBX19 | TBX5 |
| TCF4 | TCN2 | TECTA | TG | TGM1 | THRA |
| TIMM8A | TJP2 | TMC1 | TMEM11 | TMIE | TMPRSS3 |
| TPO | TPRN | TRAP1 | TRHR | TRIOBP | TRIP11 |
| TRMU | TSC1 | TSC2 | TSHB | TSHR | TSPEAR |
| TUBB3 | UBE3A | UCP2 | UGT1A1 | UMPS | UPB1 |
| UQCC2 | UQCC3 | UQCRC2 | UROS | USH1C | USH1G |
| USH2A | VWF | WAS | WDR62 | WFS1 | WNK1 |
| WT1 | YY1 | ZAP70 | ZEB2 | | |

TABLE 14
Hypotonia Gene Panel

| Gene | Disorder |
|---|---|
| AARS2 | Combined [O] Phosphorylation Deficiency 8 |
| AASS | Hyperlysinemia (Saccharopinuria) |
| ABCC8 | Hyperinsulinemic Hypoglycemia 1 |
| ABCC9 | Cantu Syndrome |
| ABCD4 | Methylmalonic/Homocystinuria CblJ |
| ACADM | Non-Ketotic Hyperglycinemia |
| ACADS | Non-Ketotic Hyperglycinemia |
| ACOX1 | Pseudoneonatal Adrenoleukodystrophy |
| ACTA1 | Nemaline Myopathy 3 |
| ACTB | Baraitser-Winter Syndrome 1 |
| ADAT3 | Mental Retardation AR 36 |
| ADCK3 | Coenzyme Q10 Deficiency 4 |
| ADK | Hypermethioninemia |
| ADNP | Mental Retardation Syn AD 28 |
| ADSL | Progressive Neonatal Encephalopathy |
| AGK | Mitochondrial DNA Deletion Syn 10 (Sengers Syndrome) |
| AGL | Glycogen Storage Disease Type 3 A/B |
| AHCY | Hypermethioninemia |
| AHI1 | Joubert Syndrome 3 |
| AIFM1 | Combined [O] Phosphorylation Deficiency 6 |
| ALDH5A1 | 4-OH-Butyric Aciduria |
| ALDH7A1 | Pyridoxine Dependent Epilepsy |
| ALDH18A1 | Cutis Laxa 3A; Hyperammonemia |
| ALG1 | Cong. Disorder of Glycosylation 1K |
| ALG2 | Cong. Disorder of Glycosylation 1I |
| ALG3 | Cong. Disorder of Glycosylation 1D |
| ALG6 | Cong. Disorder of Glycosylation 1C |
| ALG8 | Cong. Disorder of Glycosylation 1H |
| ALG9 | Cong. Disorder of Glycosylation 2, 1L |
| ALG11 | Cong. Disorder of Glycosylation 1P |
| ALG12 | Cong. Disorder of Glycosylation 1G |
| ALG13 | Cong. Disorder of Glycosylation 1S |
| ALPL | Hypophosphatasia 1 |
| AMER1 | Osteopathia Striata Congenita |
| AMPD1 | Myoadenylate Deaminase Deficiency |
| AMT | Non-Ketotic Hyperglycinemia 2 |
| | (Glycine Encephalopathy) |
| AP4B1 | Spastic Paraplegia 47 |
| AP4E1 | Spastic Paraplegia 51 |
| AP4M1 | Spastic Paraplegia 50 |
| AP1S1 | MEDNIK Syndrome |
| APOPT1 | Mitochondrial Complex IV Deficiency |
| ARHGAP31 | Adams-Oliver Syndrome 1 |
| ARID1B | Coffin-Siris Syndrome |
| ARL13B | Joubert syndrome 8 |
| ARSA | Metachromatic Leukodystrophy |
| ARX | Epileptic Encephalopathy 1 |
| ASL | Argininosuccnic Aciduria |
| ASNS | Asparagine Synthetase Deficiency |
| ASPA | Canavan Disease (Acetylaspartic Aciduria) |
| ATCAY | Cerebellar Ataxia Cayman Type |
| ATIC | AICA Ribosiduria |
| ATP5A1 | Combined Oxidative Phosphorylase Def 22 |
| ATP6VOA2 | Cutis Laxa 2A |
| ATP7A | Menkes Disease |
| ATPAF2 | Mitochondrial Complex V |
| B3GALNT2 | Muscular Dystrophy- Dystroglycanopathy A11 |
| B3GALT6 | Spondyloepimetaphyseal Dysplasia 1 |
| B3GAT3 | Multiple Joint Dislocation Syndrome |
| B4GALT1 | Congital Disorder of Glycosylation 2D |
| BCKDHA | Maple Syrup Urine Disease 1a |
| BCKDHB | Maple Syrup Urine Disease 1b |
| BCS1L | Leigh Syndrome; GRACILE Syndrome |
| BIN1 | Centronuclear Myopathy 3 |
| BMP1 | Osteogenesis Imperfecta 8 |
| BMP4 | Microphthalmia Syndrome 6 |
| BMPER | Diaphanospondylodysostosis |
| BRAF | Cardiofaciocutaneous Syndrome |
| BRP44L | Mitochondrial Pyruvate Carrier Def |
| BSND | Bartter Syndrome 4A |
| BTD | Late Onset Multiple Carboxylase Def |
| BUB1B | Mosaic Varigated Aneuploidy Syndrome 1 |
| C2orf25 | Methylmalonic Aciduria CblD |
| C5orf42 | Joubert Syndrome 18 |
| C10orf2 | Mitochondrial DNA Depletion Syn 7 |
| C12orf57 | Temamy Syndrome |
| C12orf65 | Combined Oxidative Phosphorylase Def 7 |
| CAMTA1 | Cerebellar Ataxia |
| CANT1 | Desbuquois Dysplasia |
| CASK | FG Syndrome 4 |
| CASR | Hyperparathyroidism |
| CC2D2A | COACH Syndrome; Joubert Syndrome 9 |
| CCDC78 | Centronuclear Myopathy 4 |
| CDKL5 | Epileptic Encephalopathy 2 |
| CEP41 | Joubert Syndrome 15 |
| CEP57 | Mosaic Varigated Aneuploidy Syn 2 |
| CEP290 | Joubert Syndrome 5; Meckel Syn 4 |
| CFL2 | Nemaline Myopathy 7 |
| CHAT | Congenital Myasthenic Syn 1A2 |
| CHKB | Congenital Muscular Dystrophy 1E |
| CHRNA1 | Congenital Myasthenic Syn 2 |
| CHRNB1 | Congenital Myasthenic Syn 2B |
| CHRND | Congenital Myasthenic Syn 2 |
| CHRNE | Congenital Myasthenic Syn 2E |
| CHST14 | Ehlers-Danlos Syndrome 1 |
| CNTN1 | Compton North Myopathy |
| COA5 | Mitochondrial Complex IV Deficiency |
| COG1 | Congenital Disorder of Glycosylation 2G |
| COG4 | Congenital Disorder of Glycosylation 2J |
| COG5 | Congenital Disorder of Glycosylation 2i |
| COG6 | Congenital Disorder of Glycosylation 2l |
| COG7 | Congenital Disorder of Glycosylation 2e |
| COG8 | Congenital Disorder of Glycosylation I2h |
| COL1A1 | Ehlers-Danlos Syndrome 1C, 7A |
| COL1A2 | Ehlers-Danlos Syndrome 7A2, 7B, 11 |
| COL2A1 | Spondyloepiphyseal Dysplasia Congenita |
| COL5A1 | Ehlers-Danlos 1,2 |
| COL5A2 | Ehlers-Danlos 1B, 2 |
| COL6A1 | Ullrich Cong Muscular Dystrophy 3 |
| COL6A2 | Ullrich Cong Muscular Dystrophy 1 |
| COL6A3 | Ullrich Cong Muscular Dystrophy 2 |
| COL18A1 | Mitochondrial Complex IV Deficiency |
| | (Knobloch Syndrome) |
| COLQ | Congenital Myasthenic Syndrome 1C |
| COQ2 | Coenzyme Q10 Deficiency 1 |
| COG9 | Coenzyme Q10 Deficiency 5 |
| COX6B1 | Mitochondrial Complex IV Deficiency |
| COX10 | Mitochondrial Complex IV Deficiency |
| | (Leigh Syndrome) |
| COX14 | Mitochondrial Complex IV Deficiency |
| COX15 | Leigh Syndrome |
| COX20 | Mitochondrial Complex IV Deficiency |
| CPT1A | Non-Ketotic Hypoglycinemia |
| CPT2 | Non-Ketotic Hypoglycinemia |
| CREBBP | Rubenstein-Taybi syndrome |
| CSPP1 | Joubert syndrome 22 |
| CTCF | Mental Retardation AD 21 |
| CTNNB1 | Mental Retardation AD Syndrome 19 |
| CWF19L1 | Spinocerebellar Ataxia 17 |
| D2HGDH | D-2-Hydroxyglutaric Aciduria |
| DBH | Neurotransmitter Defect |
| DCHS1 | Van Maldergren Syndrome 1 |
| DDC | Neurotransmitter Defect |
| DDOST | Congenital Disorder of Glycosylation 1R |
| DDR2 | Spondylometaepiphyseal Dysplasia 5 |
| DGKD | Diacylglycerol Kinase Deficicency |
| DGUOK | Mitochondrial DNA Depletion Syn 3 |
| DHCR7 | Smith-Lemli-Opitz Syndrome |
| DHFR | Megaloblastic Anemia |
| DIS3L2 | Perlman Syndrome |
| DLAT | Lactic Acidemia (Pyruvate Dehydrogenase E2) |
| DLD | Maple Syrup Urine Disease Type 3 |
| DMPK | Centronuclear Myopathy |
| DMWD | Dystrophia Myotonica |
| DNM2 | Charcot-Marie-Tooth Syndrome 2M, B |
| DOCK6 | Adams-Oliver Syndrome 2 |
| DOLK | Congenital Disorder of Glycosylation 1M |
| DPAGT1 | Congenital Disorder of Glycosylation 1G |
| DPM1 | Congenital Disorder of Glycosylation 1E |
| DPM2 | Congenital Disorder of Glycosylation 1U |
| DPYD | Thymine-Uraciluria |
| DST | Sensory and Autonomic Neuropathy 6 |
| DYSF | Limb-Girdle Muscular Dystrophy 2B |
| EARS2 | Combined Oxidative Phosphorylase Def 12 |
| EBP | Chondrodysplasia Punctata 2 |
| | (Conradi-Hünermann Syndrome) |
| EFEMP2 | Cutis Laxa 1B |
| EFNB1 | Craniofrontonasal Dysplasia |
| EGR2 | Dejerine-Sottas Disease |
| ELAC2 | Combined Oxidative Phosphorylase Def 17 |
| EMX2 | Schizencephaly |
| EPG5 | Vici Syndrome |
| ERCC6 | Cerebrooculofacioskeletal Syndrome 1 |
| ERCC6L2 | Bone Marrow Failure Syndrome |
| ETFA | Glutaric Acidemia Type 2A |
| | (Multiple Acyl-CoA Dehydrogenase) |
| ETFB | Glutaric Acidemia Type 2B |
| | (Multiple Acyl-CoA Dehydrogenase) |
| ETFDH | Glutaric Acidemia Type 2C |
| EXOSC3 | Pontocerebellar Hypoplasia 1B |
| EZH2 | Weaver Syndrome |
| FAH | Tyrosinemia 1 - Hepatorenal |
| FAM111A | Gracile Bone Dysplasia |
| FAM126A | Hypomyelinating Leukodystrophy 5 |
| FAR1 | Rhizomelic Chondrodysplasia Punctata 4 |
| FARS2 | Combined Oxidative Phosphorylase Def 14 |
| FASTKD2 | Mitochondrial Complex IV Deficiency |
| FAT4 | Van Maldergren Syndrome 2 |
| FBP1 | Fructose-1,6-Biphosphatase Def |
| FBXL4 | Mitochondrial DNA Depletion Syndrome 13 |
| FH | Fumarate Hydrotase Deficiency |
| FIG4 | Yunis-Varon Syndrome |
| FGFR1 | Hartsfield syndrome |
| FKBP14 | Ehlers-Danlos Syndrome 6C |
| FKRP | Muscular Dystrophy Dystroglycanopathy A10 |
| FKTN | Muscular Dystrophy Dystroglycanopathy A4 |
| | (Walker Warburg Syndrome) |
| FLNA | FG Syndrome 2 |
| FOXG1 | Congenital Rett-like Syndrome 1 |
| FOXRED1 | Mitichondrial Complex I Def (Leigh Syn) |
| GALE | Galactosemia 3 |
| GALT | Galactosemia 1 |
| GAMT | Creatine Deficiency Syndrome 2 |
| | (Muscle Hypotonia Encephalopathy) |
| GBE1 | Glycogen Storage Disease Type 4 |
| | (Andersen Disease) |
| GCDH | Glutaric Aciduria 1 |
| GCH1 | Hyperphenylalaninemia |
| | (Biopterin Cofactor Defect B) |
| GCK | MODY 2 (Hyperinsulinism) |
| GCSH | Glycine Encephalopathy |
| | (Non-Ketotic Hyperglycinemia) |
| GFER | Combined Mitochondrial Complex Def |
| GFM1 | Combined Oxidative Phosphorylase Def 1 |
| GJC2 | Hypomylinating Leukodystrophy 2 |
| GLB1 | GM1-Gangliosidosis 1, 2, 3 (Mucopolysaccharidosis 4B) |
| GLDC | Glycine Encephalopathy (Non-Ketotic Hyperglycinemia) |
| GLUL | Congenital Glutamine Deficiency |
| GLYCTK | D-Glyceric Aciduria |
| GMPPB | Muscular Dystrophy Dystroglycanopathy A14 |
| GNPAT | Rhizomelic Chondrodysplasia Punctata 2 |
| | (Costello Syndrome) |
| GNPTAB | Mucolipidosis 3A |
| GPC3 | Simpson-Golabi-Behmel Syndrome 1 |
| GPHN | Molybdenum Cofactor Deficiency C |
| GRIN2B | Epileptic Encephalopathy 27 |
| GRM1 | Spinocerebellar Ataxia 13 |
| HADH | Hyperinsulinemic Hypoglycemia 4 |
| | (Non-Ketotic Hypoglycemia) |
| HADHA | Trifunctional Protein Deficiency α |
| HADHB | Trifunctional Protein Deficiency β |
| HEXA | Tay-Sachs Disease |
| HEXB | Sandhoff Disease |
| HIBCH | 3-OH Isobutyric Aciduria |
| HLCS | Multiple CoA Carboxylase Deficiency (Biotin Responsive) |
| HPRT1 | Lesch Nyhan Disease |
| HRAS | Costello Syndrome |
| HSD17B4 | D-Bifunctional Protein Deficiency |
| HSD17B10 | 2-Methyl-3-OH-Butyric Aciduria |
| HSPD1 | Hypomyelinating Leukodystrophy 4 |
| IFIH1 | Aicardi-Goutieres Syndrome 7 |
| IFT122 | Cranioectodermal Dysplasia 1 |
| IKBKAP | Familial Dysautonomia |
| IMPDH1 | Leber Congenital Anaurosis 11 |
| INPP5E | Joubert Syndrome 1 |
| INPPL1 | Opismodysplasia |
| INS | MODY Type 10 |
| ISPD | Muscular Dystrophy Dystroglycanopathy A7 |
| ITGA7 | Congenital Muscular Dystrophy |
| ITPR1 | Spinocerebellar Ataxia 29 |
| KANK1 | Cerebral Palsy 2 |
| KAT6B | SBBYSS Syndrome; Ohdo Syndrome |
| KCNJ11 | Hyperinsulinic Hypoglycemia 1, 2 |
| KCNK9 | Birk-Barel Syndrome |
| KCNQ2 | Epileptic Encephalopathy 7 |
| KCTD1 | Scalp Ear Nipple Syndrome |
| KDM6A | Kabuki Syndrome 3 |
| KIAA0196 | Spastic Paraplegia 8 |
| KIAA1279 | Goldberg-Shprintzen Syndrome |
| KIF7 | Joubert Syndrome 12 |
| KIF1A | Spastic Paraplegia 30 |
| KIF11 | Microcephaly-Lymphedema-MR Syn 2 |
| KIF22 | Spondyloepimetaphyseal Dysplasia 2 |
| KPTN | Mental Retardation AR 41 |
| LAMA2 | Congenital Muscular Dystrophy 1A |
| LAMB2 | Pierson Syndrome |
| LARGE | Muscular Dystrophy Dystroglycanopathy A6 |
| LIAS | Lipoic Acid Synthetase Deficiency |
| LIFR | Syuve-Wiedemann Syndrome |
| LMBRD1 | Methylmalonic/Homocystinuria CblF |
| LMNA | Congenital Muscular Dystrophy |
| LMOD3 | Nemaline Myopathy |
| LRP5 | Osteoporosis-Pseudoglioma Syndrome |
| LRPPRC | Leigh Syndrome - French Canadian |
| LYRM4 | Combined Oxidative Phosphorylase Def 19 |
| MAGEL2 | Prader-Willi Syndrome |
| MANBA | β-Mannosidosis |
| MAP2K1 | Cardiofaciocutaneous Syndrome 3 |
| MAP2K2 | Craniofaciocutaneous Syndrome 4 |
| MCCC1 | 3-Methylcrotonylglycinuria 1 |
| MCOLN1 | Mucolipidosis IV |
| MECP2 | Neonatal Encephalopathy; Rett Syndrome |
| MED12 | Lujan-Fryns Syndrome |
| MEGF10 | Myopathy-Areflexia-RDS-Dysphagia Syn |
| MGAT2 | Congenital Disorder of Glycosylation 2A |
| MLYCD | Malonic Aciduria |
| MMACHC | Methylmalonic/Homocystinuria, cblC |
| MMAB | Methylmaloic Aciduria CblB Def |
| MOGS | Congenital Disorder of Glycosylation 2B |
| MPDU1 | Congenital Disorder on Glycosylation 1F |
| MPI | Congenital Disorder of Glycosylation 1B |
| MPV17 | Mitochondria] DNA Depletion Syn 6 |
| MPZ | Cong Hypomyelinating Neuropathy |
| MRPL3 | Combined Oxidative Phosphorylase Def 9 |
| MRPL44 | Combined Oxidative Phosphorylase Def 16 |
| MRPS16 | Combined Oxidative Phosphorylase Def 2 |
| MRPS22 | Combined Oxidative Phosphorylase Def 5 |
| MTFMT | Combined Oxidative Phosphorylase Def 15 |
| MTHFR | Homocystinuria |
| MTM1 | Myotubular Myopathy 1 |
| MTO1 | Combined Oxidative Phosphorylase Def 10 |
| MTR | Methylmalonic/Homocystinuria CblG |
| MUSK | Congenital Myastheric Syndrome 1D |
| MUT | Methylmalonic Aciduria Type 0 |
| MVK | Mevalonic Aciduria |
| NAA10 | N-Terminal Acyltransferase Deficiency |
| NADK2 | 2,4-Dienoyl-CoA Reductase Def |
| NAGA | Schindler Disease 1, 3 |
| NALCN | Neuroaxonal Degeneration |
| NDN | Prader-Willi Syndrome |
| NDUFA1 | Mitochondrial Complex I Def |
| NDUFA2 | Mitochondrial Complex I Def (Leigh Syn) |
| NDUFA8 | Mitochondrial Complex I Def |
| NDUFA9 | Mitochondrial Complex I Def (Leigh Syn) |
| NDUFA10 | Leigh Syndrome |
| NDUFA11 | Mitochondrial Complex I |
| NDUFA19 | Mitochondrial Complex I Def (Leigh Syn) |
| NDUFAF1 | Mitochondrial Complex I Def |
| NDUFAF2 | Mitochondrial Complex I Def (Leigh Syn) 3 |
| NDUFAF3 | Mitochondrial Complex I Def 6 |
| NDUFAF4 | Mitochondrial Complex I Def |
| NDUFAF5 | Mitochondrial Complex I Def |
| NDUFAF6 | Mitochondrial Complex I Def (Leigh Syn) |
| NDUFB3 | Mitochondrial Complex I Def |
| NDUFB9 | Mitochondrial Complex I Def |
| NDUFS1 | Mitochondrial Complex I Def |
| NDUFS2 | Mitochondrial Complex I Def |
| NDUFS3 | Mitochondrial Complex I Def (Leigh Syn) |
| NDUFS4 | Mitochondrial Complex I Def 1 (Leigh Syndrome) |
| NDUFS6 | Mitochondrial Complex I Def 2 |
| NDUFS7 | Leigh Syndrome |
| NDUFS8 | Mitochondrial Complex I Def (Leigh Syn) |
| NDUFV1 | Mitochondrial Complex I Def (Leigh Syndrome) |
| NDUFV2 | Mitochondrial Complex I Def |
| NEB | Nemaline Myopathy 2 |
| NEU1 | Sialidosis (Mucolipidosis) 1, 2 |
| NFIX | Marshall-Smith Syndrome |
| NGLY1 | Congenital Defect in Glycosylation Iv |
| NKX2-1 | Congenital Hypothyroidism (Goiterous) |
| NPC1 | Niemann-Pick Type C1, D |
| NPC2 | Niemann-Pick Type C2 |
| NPHP1 | Joubert Syndrome 4 |
| NRAS | Noonan Syndrome 6 |
| NRXN1 | Pitt-Hopkins Syndrome 2 |
| NSD1 | Sotos Syndrome 1 (Beckwith-Wiedemann Syndrome) |
| NSDHL | CK Syndrome |
| NUBPL | Mitochondrial Complex I Deficiency |
| OCLN | Band Like Calcification |
| OFD1 | Simpson-Golabi-Behmel Syndrome 2 Joubert Syndrome 10 |
| OGDH | α-Ketoglutaric Aciduria |
| OCRL | Lowe Syndrome |
| OPHN1 | MR Cerebellar Hypoplasia Syndrome 60 |
| ORAI1 | Immunodeficiency 9 |
| OTX2 | Micropthalmia Syndrome 5 |
| PC | Lactic Acidemia |
| PCCA | Propionic Aciduria |
| PCCB | Propionic Aciduria |
| PDE6D | Joubert Syndrome 22 |
| PDHA1 | Lactic Acidemia (Leighs Syndrome) |
| PDHB | Lactic Acidemia |
| PDHX | Leighs Syndrome; Lactic Acidemia |
| PDP1 | Lactic Acidemia |
| PDSS1 | Coenzyme Q10 Deficiency 2 |
| PDSS2 | Coenzyme Q10 Deficiency 3 |
| PDX1 | MODY Type 4 (Lactic Acidemia) |
| PET100 | Mitochondrial Complex IV Deficiency |
| PEX1 | Peroxisome Biogenesis Disorder 1A, 1B |
| | Neonatal Adrenal Leukodystrophy |
| | Zellweger Syndrome |
| PEX2 | Peroxisome Biogenesis |
| | Disorder 5A, 5B Zellweger Syndrome |
| PEX3 | Peroxisome Biogenesis Disorder 10A |
| | Zellweger Syndrome 6 |
| PEX5 | Peroxisome Biogenesis Disorder 6A, 6B |
| | Zelwegers Syndrome; Infantile Refsum |
| | Neonatal Adrenal Leukodystrophy 2 |
| PEX6 | Peroxisome Biogenesis Disorder 2A, 2B |
| | Zellweger Syndrome Neonatal Adrenal |
| | Leukodystrophy |
| PEX7 | Peroxisome Biogenesis Disorder 9B Rhizomelic |
| | Chondrodysplasia 1 |
| PEX10 | Peroxisome Biogenesis Disorder 6A, 6B |
| | Zellweger Syndrome Neonatal Adrenal |
| | Leukodystrophy |
| PEX11B | Peroxisome Biogenesis Disorder 14B |
| PEX12 | Peroxisome Biogenesis Disorder 3A, 3B |
| | Zellweger Syndrome Neonatal Adrenal |
| | Leukodystrophy Infantile Refsums |
| PEX13 | Peroxisome Biogenesis Disorder 11A, 11B |
| | Zellweger Syndrome Neonatal Adrenal |
| | Leukodystrophy |
| PEX14 | Peroxisome Biogenesis Disorder 13A |
| | Zellweger Syndrome |
| PEX16 | Peroxisome Biogenesis Disorder 8A, 8B |
| | Zellweger Syndrome |
| PEX19 | Peroxisome Biogenesis Disorder 12A |
| | Zellweger Syndrome 5 |
| PEX26 | Peroxisome Biogenesis Disorder 7A, 7B |
| | Zellweger Syndrome |
| PGAP1 | Mental Retardation AD Syn 42 |
| PGM3 | Congenital Disorder of Glycosylation 2M |
| | (Immunodeficiency 23; Hyper IgE Syn) |
| PIEZO2 | Marden-Walker Syndrome |
| PIGA | Multiple Cong Anomalies-Hypotonia-Seizure Syn 2 |
| PIGL | CHIME Syndrome |
| PIGN | Multiple Cong Anomalies-Hypotonia-Seizure Syn 1 |
| PIGO | Hypophosphatasia Mental Retardation Syn 2 |
| PIGT | Multiple Cong Anomalies-Hypotonia-Seizure Syn 3 |
| PIK3CA | Megalencephaly-Capillary Syndrome |
| PLA2G6 | Neuronaxonal Dystrophy 1 |
| PLG | Dysplasminogenemic Thrombosis |
| PLOD1 | Ehlers-Danlos Type VI |
| PLP1 | Pelizaeus-Merzbacher Disease 1 |
| PMM2 | Congenital Disorder of Glycosylation 1A |
| PMP22 | Dejerine-Sottas Disease 1 |
| PNPO | Epileptic Encephalopathy |
| PNPT1 | Combined Oxidative Phosphorylase Def 13 |
| POLG | Mitochondrial DNA Depletion Syn 4A, 4B |
| POLR3B | Hypomyelinating Leukodystrophy 8 |
| POMGNT1 | Muscular Dystrophy Dystroglycanopathy A3 |
| POMGNT2 | Muscular Dystrophy Dystroglycanopathy A8 |
| POMK | Muscular Dystrophy Dystroglycanopathy A12 |
| POMT1 | Muscular Dystrophy Dystroglycanopathy A2 |
| POMT2 | Muscular Dystrophy Dystroglycanopathy A6 |
| POU1F1 | Congenital Hypothyroidism |
| | (Combined Pituitary Hormone Deficiency) |
| PRKAG2 | Glycogen Storage Disease (Heart) |
| PRODH | Hyperprolinemia Type 1 |
| PRPS1 | Charcot-Marie-Tooth 5 |
| PRX | Dejerine-Sottas Syndrome |
| PSAP | Metachromatic Leukodystrophy |
| | (Combined Saposin Deficiency) |
| PTDSS1 | Lenz-Mejewski Hyperotitic Syndrome |
| PTEN | Bannayan-Riley-Ruvalcaba Syndrome |
| PTS | Hyperphenylalaninemia (Biopterin Cofactor Defect A) |
| PURA | Mental Retardation AD Syndrome 31 |
| | (Chromosome 5q31.3 Microdeletion) |
| PXDN | Corneal Opacification |
| QDPR | Hyperphenylalaninemia (Biopterin Cofactor Defect C) |
| RAB3GAP1 | Warburg Micro Syndrome 1 |
| RAB3GAP2 | Warburg Micro Syndrome 2 |
| RAB18 | Warburg Micro Syndrome 3 |
| RAPSN | Congenital Myosthenic Syndrome |
| RBM10 | TARP Syndrome |
| RELN | Lissencephaly 2 (Norman Roberts Syn) |
| RFT1 | Congenital Disorder of Glycosylation 1N |
| RMND1 | Combined Oxidative Phosphorylase Def 11 |
| RPGRIP1L | COACH Syndrome 2; Joubert Syn 7 |
| RRM2B | Mitochondrial DNA Depletion Syn 8A, 8B |
| RYR1 | Minicore Myopathy |
| SBDS | Shwachman-Bodian-Diamond Syndrome |
| SC5DL | Lathosterolosis |
| SCO2 | Cardioencephalomyopathy 1 |
| SDHA | Leigh Syndrome |
| SDHAF1 | Mitochondrial Complex II Deficiency |
| SEPN1 | Congenital Myopathy Rigid Spine 1 |
| SERAC1 | 3-Methyl-Glutaconic Aciduria |
| SFXN4 | Combined Oxidative Phosphorylase Def 18 |
| SHH | Schizencephaly |
| SIL1 | Marinesco-Sjogren Syndrome |
| SIX3 | Schizencephaly |
| SKI | Shprintzen-Goldberg Syndrome |
| SLC6A3 | Parkinsonism Dystonia - Infantile |
| | (Attention Defecit Disorder) |
| SLC6A8 | Creatine Deficiency 1 |
| SLC7A7 | Lysinuric Protein Intolerance |
| SLC12A6 | Agenesis of Corpus Callosum |
| SLC16A2 | Allan-Herndon-Dudley Syndrome |
| SLC17A5 | Sialic Storage Disease, Sallas Disease |
| SLC22A5 | Primary Systemic Carnitine Deficiency |
| SLC25A1 | Combined D-2, L-2 OH Glutaric Aciduria |
| SLC25A15 | HHH Syndrome |
| SLC25A19 | Amish Microcephaly |
| SLC25A20 | Carnitine Translocase Deficiency |
| SLC25A22 | Epileptic Encephalopathy 3 |
| SLC33A1 | Cataracts, Hearing Loss Neorogeneration Syn |
| SLC35A2 | Congenital Disorder of Glycosylation 2M |
| SLC35C1 | Congenital Disorder of Glycosylation 2C |
| SLC46A1 | Congenital Folate Malabsorption Syndrome |
| SMARCB1 | Mental Retardation AD Syndrome 15 |
| SMN1 | Spinal Muscular Atrophy |
| SMPD1 | Nieman-Pick Disease A, B |
| SNIP1 | Craniofacial Dysmorphism |
| SNRPN | Prader-Willi Syndrome |
| SOX2 | Micropthalmia Syndrome 3 |
| SOX9 | Campomelic Dysplasia |
| SOX10 | Wardenburg Syndrome |
| SOX17 | Vesicoureteric Reflux CALUT Syn 3 |
| SPEG | Centronuclear Myopathy 5 |
| SPR | Hyperphenylalaninemia (Biopterin Cofactor Defect) |
| | (DOPA Responsive Dystonia) |
| SPTBN2 | Spinocerebellar Ataxia 5, 14 |
| SPTLC1 | Sensory & Autonomic Neuropathy 1A |
| SRD5A3 | Congenital Disorder of Glycosylation Iq |
| SSR4 | Congenital Disorder of Glycosylation Iy |
| STAMBP | Microcephaly-Capillary Malformation Syn |
| STIM1 | Immunodeficiency 10 |
| STRA6 | Microphthalmia Syndrome 9 |
| STT3A | Congenital Disorder of Glycosylation 1A |
| STT3B | Congenital Disorder of Glycosylation 1Y |
| STXBP1 | Epileptic Encephalopathy 4 |
| SUCLA2 | Mitochondrial DNA Depletion Syn 5 |
| SUCLG1 | Mitochondrial DNA Depletion Syn 9 |
| | (Methylmalonic Aciduria) |
| SUMF1 | Multiple Sulfatase Deficiency |
| SURF1 | Leighs Syndrome; Cox Deficiency |
| SYNE1 | Emery-Dreifuss Muscular Dystrophy 4 |
| SYNGAP1 | Mental Retardation AD Syn 5 |
| TACO1 | Mitochondrial Complex IV Deficiency |
| | (Leighs Syndrome) |
| TARS2 | Combined Oxidative Phosphorylase Def 21 |
| TBC1D20 | Warburg Micro Syndrome 3 |
| TBC1D24 | DOOR Syndrome |
| TCN2 | Methylmalonic/Homocystinuria |
| TCTN3 | Joubert Syndrome 18 |
| TECT1 | Joubert Syndrome 13 |
| TH | Segawa Syndrome |
| TGFB3 | Rienhoff Syndrome |
| TMCO1 | Craniofacial Dysmorphism |
| TMEM5 | Muscular Dystrophy Dystroglycanopathy A10 |
| TMEM67 | COACH Syndrome; Joubert Syndrome 6 |
| TMEM70 | Mitochondrial Complex V Deficiency 2 |
| TMEM138 | Joubert Syndrome 16 |
| TMEM165 | Congenital Disorder of Glycosylation 2K |
| TMEM216 | Joubert Syndrome 2 |
| TMEM231 | Joubert Syndrome 20 |
| TMEM237 | Joubert Syndrome 14 |
| TNXB | Ehlers-Danlos Syndrome |
| TPI1 | Hemolytic Anemia |
| TPM2 | Nemaline Myopathy 4 |
| TPM3 | Nemaline Myopathy 1 Mental Retardation AR Syn 13 |
| TREX1 | Aicardi-Goutieres Syndrome 1 |
| TRMU | Transient Liver Failure |
| TRNT1 | Sideroblastic Anemia |
| TSFM | Combined [O] Phosphorylation Defect 3 |
| TSHB | Congenital Hypothyroidism (Non-Goiterous 4) |
| TTN | Early Myopathy with Cardiopathy |
| TUBA1A | Lissencephaly 3 |
| TUBA8 | Polymicrogyria |
| TUBB2B | Polymicrogyria |
| TUBB3 | Cortical Dysplasia 1 |
| TUBB4A | Hypomyelinating Leukodystrophy 6 |
| TUBGCP6 | Microcephaly Chorioretinopathy 1 |
| TUFM | Combined Oxidative Phosphorylase Def 4 |
| UBA1 | Spinal Muscular Atrophy |
| UBE3B | Blepharophimosis-Ptosis Syndrome |
| UBR1 | Johanson-Blizzard Syndrome |
| UPB1 | N-Carbamyl-β-Aminoaciduria |
| UQCC2 | Mitochodrial Complex III Deficiency 7 |
| USP9X | Mental Retardation Syndrome 99 |
| VARS2 | Combined Oxidative Phosphorylase Def |
| VLDLR | Cerebellar Hypoplasia-MR Syndrome 1 |
| VMA21 | Myopathy with Autophagia |
| VPS13B | Cohen Syndrome |
| VPS33B | Arthrogryposis-Renal-Cholestasis Syn 1 |
| VPS53 | Pontocerabellar Hypoplasia 2E |
| WDR19 | Cranioectodermal Dysplasia 4 |
| WNK1 | Pseudohypoaldosteronism 2C |
| ZBTB20 | Primrose Syndrome |
| ZC4H2 | Wieacker-Wolf Syndrome |
| ZEB2 | Mowat-Wilson Syndrome |
| ZNF423 | Joubert Syndrome 19 |

TABLE 15
Inborn Error NICU Mutations Panel

| GENE | DISORDER |
|---|---|
| AASS | Hyperlysinuria (Saccharopinuria) |
| ABAT | GABA-Transacylase Deficiency |
| ABCB4 | Progressive Intrahepatic Cholestasis 3 |
| ABCB11 | Progressive Intrahepatic Cholestasis 2 |
| ABCC2 | Dubin-Johnson Syndrome |
| ABCC8 | Hyperinsulinemia |
| ABCD1 | X-Linked Adrenoleukodystrophy |
| ABCD3 | Zellweger Syndrome 2 |
| ABCG8 | Sitosterolemia |
| ACAA1 | Pseudo-Zellweger Syndrome |
| ACAD8 | Isobutyryl-CoA Dehydrogenase |
| ACAD9 | Dicarboxylic Aciduria |
| ACADL | Non-Ketotic Hypoglyemia |
| ACADM | Non-Ketotic Hypoglycemia |
| ACADS | Non-Ketotic Hypoglycemia |
| ACADSB | 2-Methylbutyryl Glycinuria |
| ACADVL | Non-Ketotic Hypoglycemia |
| ACAT1 | α-Methylacetoacetic Aciduria 1 |
| | (Ketoacidosis) Pseudo-Neonatal |
| | Adrenoleukodystrophy |
| | (Peroxisomal Biogenesis Disorder) |
| ACSF3 | Malonic-Methylmalonic Aciduria |
| ADA | Severe Combined Immunodeficiency |
| ADSL | Psychomotor Retardation |
| AGA | Aspartylglucosaminuria |
| AGL | Glycogen Storage Disease Type 3 |
| AGPS | Rhizomelic Chondrodysplasia Punctata 3 |
| AGXT | Primary Hyperoxaluria Type 1 |
| | (Glycolic Aciduria) |
| AHCY | Hypermethioninemia |
| AIRE-1 | Autoimmune Polyglandular Disease 1 |
| AKAP9 | Long QT Syndrome 2 |
| AKR1D1 | Congenital Bile Acid Synthesis Defect 2 |
| AKT2 | Hypoinsulinemia |
| ALAD | Acute Hepatic Porphyria |
| ALAS2 | X-Linked Erythropoietic Protoporphyria |
| | (X-Linked Sideroblastic Anemia) |
| ALDH3A2 | Sjögren-Larsson Syndrome |
| ALDH4A1 | Hyperprolinemia Type 2 |
| ALDH5A1 | 4-Hydroxybutyric Aciduria |
| ALDOA | Glycogen Storage Disease Type 12 |
| ALDOB | Hereditary Fructose Intolerance |
| ALG3 | Cong. Disorder of Glycosylation 1D |
| ALG6 | Cong. Disorder of Glycosylation 1C |
| ALG9 | Cong. Disorder of Glycosylation 2 |
| AMACR | Congenital Bile Acid Synthesis Defect 4 |
| | (Peroxisomal Biogenesis Disorder) |
| AMPD1 | Myopathy |
| AMT | Non-Ketotic Hyperglycinemia |
| ANK2 | Long QT Syndrome 4 |
| APOC2 | Hyperlipoproteinemia Type 1B |
| APOE | Dysbetalipoproteinemia (Hyperlipoproteinemia 3) |
| APRT | 2,8-Dihydroxyadenine Urolithiasis |
| ARG1 | Argininemia |
| ARSA | Metachromatic Leukodystrophy |
| ARSB | Maroteaux-Lamy Syndrome |
| | (Mucopolysaccharidosis Type VI) |
| ARSE | Chondrodysplasia Punctata 1 |
| ASL | Argininosuccinic Aciduria |
| ASPA | Canavan Disease (Acetylaspartic Aciduria) |
| ASS1 | CitrullinemiaType 1 |
| ATM | Ataxia Telangiectasia (Louis Barr Syndrome) |
| ATP7A | Menkes Disease |
| ATP7B | Wilson Disease |
| ATP8B1 | Progressive Intrahepatic Cholestasis 1 |
| | (Byler Disease) |
| AUH | 3-Methylglutaconic Aciduria Type 1 |
| BCAT2 | Branched Chain Aminotransferase |
| BCKDHA | Maple Syrup Urine Disease Type 1A |
| BCKDHB | Maple Syrup Urine Disease Type 1B |
| BCS1L | Mitochondrial Complex III Deficiency |
| BRAF | Leopard Syndrome 3 (Noonan Syndrome 7) |
| BTD | Late Onset Multiple Carboxylase |
| C7orf10 | Glutaric Aciduria Type 3 |
| C12orf62 | Mitochondrial Complex IV Deficiency |
| C20orf7 | Mitochondrial Complex I Def |
| CA5A | Hyperammonemia |
| CACNA1C | Long QT Syndrome 8 |
| | (Brugada Syndrome; Timothy Syndrome) |
| CAT | Acatalasemia |
| CAV3 | Long QT Syndrome 9 |
| | (Limb Girdle Muscular Dystrophy 1C) |
| CBS | Homocystinuria |
| CD40L | Immunodeficiency with Hyper IgM |
| CDKN1C | Beckwith-Wiedemann Syndrome |
| CLN3 | Neuronal Ceroid Lipofuscinosis 3 |
| | (Spielmeyer-Vogt-Batten Disease) |
| CLN5 | Neuronal Ceroid Lipofuscinosis 5 |
| | (Late Infantile - Finnish Type) |
| CLN6 | Neuronal Ceroid Lipofuscinosis 4A, 6 |
| | (Late Infantile) (Adult - Kufs Disease) |
| CLN8 | Neuronal Ceroid Lipofuscinosis 8 |
| | (Northern Epilepsy Variant) |
| CNDP1 | Carnosinemia |
| CFTR | Cystic Fibrosis |
| COA5 | Mitochondrial Complex IV Deficiency |
| COX6B1 | Mitochondrial Complex IV Deficiency |
| COX10 | Mitochondrial Complex IV Deficiency |
| COX15 | Leigh Syndrome |
| COX20 | Mitochondrial Complex IV Deficiency |
| CP | Aceruloplasminemia |
| CPOX | Coproporphyria |
| CPS1 | Hyperammonemia |
| CPT1A | Non-Ketotic Hypoglycemia |
| CPT2 | Non-Ketotic Hypoglycemia; Myopathy |
| CTH | Cystathionninuria (Benign) |
| CTNS | Cystinosis |
| CTSA | Galactosialidosis (Goldberg Syndrome) |
| CTSD | Neuronal Ceroid Lipofuscinosis 10 |
| | (Cathepsin D Deficiency) |
| CYP11B1 | Congenital Adrenal Hyperplasia 4 |
| CYP11B2 | Aldosteronism |
| CYP17A1 | Congenital Adrenal Hyperplasia 5 |
| CYP21A2 | Congenital Adrenal Hyperplasia 3 |
| CYP27A1 | Cerebrotendinous Xanthomatosis |
| D2HGDH | D-2-Hydroxyglutaric Aciduria |
| DBH | Neurotransmitter Defect |
| DBT | Maple Syrup Urine Disease Type 2 |
| DCXR | Pentosuria |
| DDC | Neurotransmitter Defect |
| DECR1 | Non-Ketotic Hypoglycemia |
| DGUOK | Mitochondrial Deoxyguanosine Kinase |
| DHCRT | Smith-Lemli-Opitz Syndrome |
| DLAT | Lactic Acidemia |
| DLD | Maple Syrup Urine Disease Type 3 |
| DNAJC5 | Neuronal Ceroid Lipofuscinosis 4B |
| | (Parry Type) |
| DNAJC19 | 3-Methyl-Glutaconic Aciduria 5 |
| DOLK | Congenital Disorder of Glycosylation 1M |
| DPYD | Thymine-Uraciluria (5-Fluorouracil Toxicity) |
| DPYS | Dihydropyrimidinuria |
| DUOX2 | Congenital Hypothyroidism (Dysmorphogenesis 6) |
| DUOXA2 | Congenital Hypothyroidism (Dysmorphogenesis 5) |
| EBP | Chondrodysplasia Punctata 2 |
| | (Conradi-Hünermann Syndrome) |
| EIF2AK3 | Wolcott-Rollison Syn (Early Onset IDDM) |
| ENO3 | Glycogen Storage Disease Type 13 |
| EPAS1 | Erythrocytosis/Polycythemia 4 |
| ETFA | Glutaric Acidemia Type 2A |
| ETFB | Glutaric Acidemia Type 2B |
| ETFDH | Glutaric Acidemia Type 2C |
| ETHE1 | Ethylmalonic Encephalopathy 1 |
| FAH | Hepatorenal Tyrosinemia Type 1 |
| FASTKD2 | Mitochondrial Encephalomyopathy |
| FBP1 | Fructose-1,6-Bisphosphatase Deficiency |
| FBXL4 | Mitochondrial DNA Depletion Syndrome 13 |
| FECH | Erythropoietic Protoporphyria |
| FOXE1 | Congenital Hypothyroidism |
| | (Bamforth-Lazarus Syndrome) |
| FOXRED1 | Mitichondrial Complex I Def (Leigh Syn) |
| FTCD | Formiminoglutamic Aciduria |
| FUCA1 | Fucosidosis 1 |
| FUCA2 | Fucosidosis 2 |
| G6PC | Von Gierke Disease (Glycogen Storage Disease Type 1A) |
| G6PD | Non-Spherocytic Hemolytic Anemia |
| GAA | Pompe Disease (Glycogen Storage Disease Type 2) |
| GALC | Krabbe Disease (Globoid Cell Leukodystrophy) |
| GALE | Galactosemia |
| GALK1 | Galactosemia |
| GALNS | Morquio A Syndrome (Mucopolysaccharidosis Type IVA) |
| GALT | Galactosemia |
| GAMT | Muscular Hypotonia; Encephalopathy |
| GBA | Gaucher Disease |
| GBE1 | Andersen Disease (Glycogen Storage Disease Type 4) |
| GCDH | Glutaric Aciduria Type 1 |
| GCH1 | Hyperphenylalaninemia (Biopterin Cofactor Defect B) |
| GCK | MODY 2 (Hyperinsulinism) |
| GCKR | Fasting Plasma Level 5 |
| GCLC | Hemolytic Anemia |
| GCSH | Non-Ketotic Hyperglycinemia |
| GGT1 | Glutathionuria |
| GH1 | Pituitary Dwarfism 1, 2 |
| GHRHR | Dwarfism |
| GK | Hyperglycerolemia/Glyceroluria |
| GLA | Fabry Disease |
| GLB1 | Morquio B Syndrome |
| | (Mucopolysaccharidosis IVB) |
| | (GM1-Gangliosidosis) |
| GLDC | Non-Ketotic Hypergylcinemia |
| GLUD1 | Hyperinsulinism/Hyperammonemia Syn |
| GLYCTK | D-Glyceric Aciduria |
| GM2A | GM2-Gangliosidosis |
| GNA11 | Hypoclacemia 2 |
| GNAS | Pseudohypoparathyroidism 1 |
| GNPAT | Rhizomelic Chondrodysplasia Punctata 2 |
| GNPTAB | Mucolipidosis IIIA |
| GNPTG | Mucolipidosis IIIC |
| GNS | Sanfilippo Disease D (Mucopolysaccharidosis IIID) |
| GPHN | Molybdenum Cofactor Defect Type C |
| GRHPR | Primary Hyperoxaluria Type 2 (Glyceric Aciduria) |
| GSS | 5-Oxoprolinuria (Pyroglutamic Aciduria) |
| GUSB | Sly Disease (Mucopolysaccharidosis VII) |
| GYG1 | Glycogen Storage Disease Type 15 |
| GYS2 | Glycogen Storage Disease Type 0 |
| H6PD | Cortisone Reductase Deficiency 2 |
| H19 | Bechwith-Wiedemann Syndrome |
| HADH | Non-Ketotic Hypoglycemia |
| HADHA | Trifunctional protein - α Subunit |
| HADHB | Trifunctional protein - β Subunit |
| HAMP | Hereditary Hemochromatosis 2B Juvenile |
| HBB | Sickle Cell Disease |
| HEXA | Tay Sachs Disease (GM2-Gangliosidosis) |
| HEXB | Sandhoff Disease |
| HESX1 | Pituitary Hormone Deficiency 5 |
| HFE | Hereditary Hemochromatosis 1 |
| | (Porphyria Varigata) |
| HGD | Alkaptonuria |
| HGSNAT | Sanfilippo Disease C |
| | (Mucopolysaccharidosis IIIC) |
| HIBCH | 3-Hydroxyisobutyric Aciduria |
| HJV | Hereditary Hemochromatosis 2A Juvenile |
| HLCS | Multiple CoA Carboxylase Deficiency |
| | (Biotin Responsive) |
| HMBS | Acute Intermittent Porphyria |
| HMGCL | 3-Hydroxy-3-Methylglutaric Aciduria Ketoacidosis |
| HMGCS2 | Ketoacidosis |
| HNF1A | MODY Type 3 |
| HNF4A | MODY Type 1 |
| HPD | Hereditary Tyrosinemia Type 3 (Hawkinsinuria) |
| HPRT1 | Lesch-Nyhan Disease |
| HSD3B2 | Congenital Adrenal Hyperplasia II |
| HSD3B7 | Progressive Intrahepatic Cholestasis 4 |
| HSD17B4 | Pseudo Neonatal Adrenoleukodystrophy |
| HSD17B10 | 2-Methyl-3-Hydroxy Butyric Aciduria |
| IDS | Hunter Syndrome (Mucopolysaccharidosis Type II) |
| IDUA | Hurler/Scheie Syndrome |
| | (Mucopolysaccharidosis Type I) |
| IGF1 | Insulin-like Growth Factor 1 Deficiency |
| IGF1R | Resistance to IGF1 |
| IKBKG | Hypohidrotic Ectodermal Dysplasia |
| INPPL1 | Opsismodysplasia |
| INS | MODY Type 10 |
| INSR | Hyperinsulinism 5 (Leprechaunism) |
| IVD | Isovaleric Acidemia |
| JAG1 | Alagille Syndrome |
| | (Cholestasis; Peripheral Pulmonary Stenosis) |
| KCNE1 | Long QT Syndrome 5 |
| | (Jervell-Lange-Nielsen Syndrome 2) |
| KCNE2 | Long QT Syndrome 6 |
| KCNH2 | Long QT Syndrome 2 |
| KCNJ2 | Long QT Syndrome 7 |
| KCNJ5 | Long QT Syndrome 7 |
| KCNJ11 | Hyperinsulinism 2 |
| KCNQ1 | Long QT Syndrome 1 |
| | (Jervell-Lange-Nielsen Syndrome 1) |
| KHK | Fructosuria (Benign) |
| L2HGDH | L-2-Hydroxyglutaric Aciduria |
| LCAT | Fish Eye Disease |
| LDHA | Glycogen Storage Disease 11 |
| LDLRAP1 | Hypercholesterolemia |
| LEP | Severe Early Onset Obesity |
| LHX3 | Congenital Hypothyroidism |
| | (Combined Pituitary Deficiency 3) |
| LHX4 | Pituitary Hormone Deficiency 4 |
| LIAS | Lipoic Acid Synthase Deficiency |
| LIPA | Wolman Disease |
| LIPC | High Density Lipoprotein Cholesterolemia |
| LMBRD1 | Methylmalonic/Homocystinuria, cblF Type |
| LMF1 | Hypertriglyceridemia |
| LPL | Hyperlipoproteinemia 1 |
| LRPPRC | Leigh Syndrome (French-Canadian) |
| MAOA | Neurotransmitter Defect (Brunner Syndrome) |
| MAN2B1 | α-Mannosidosis |
| MANBA | β-Mannosidosis |
| MAT1A | Hypermethioninemia |
| MC2R | Glucocorticol Deficiency |
| MCCC1 | 3-Methylcrotonylglycinuria 1 |
| MCCC2 | 3-Methylcrotonylglycinuria 2 |
| MCOLN1 | Mucolipidosis IV |
| MCM4 | Glucocorticol Deficiency |
| MEN1 | Multiple Endocrin Neoplasma 1 |
| MFSD8 | Neuronal Ceroid Lipofuscinosis 7 |
| MGME1 | Mitochondrial DNA Depletion Syndrome 11 |
| MLYCD | Malonic Aciduria |
| MMAA | Methylmalonic Aciduria, cblA Type |
| MMAB | Methylmalonic Aciduria, cblB Type |
| MMACHC | Methylmalonic/Homocystinuria, cblC |
| MMADHC | Methylmalonic/Homocystinuria, cblD |
| MOCS1 | Molybdenum Cofactor Defect Type A |
| MOCS2 | Molybdenum Cofactor Defect Type B |
| MPC1 | Mitochondrial Pyruvate Carrier 1 Defic |
| MPI | Congenital Disorder of Glycosylation 1B |
| MPV17 | Mitochondrial DNA Depletion Syn 6 |
| MRAP | Glucocorticoid Deficiency 2 |
| MTHFR | Homocystinuria |
| MTO1 | Oxidative Phosphorylation Deficiency 10 |
| MTR | Methylmalonic/Homocystinuria, cblG |
| MTRR | Homocystinuria (Megaloblastic Anemia) |
| MUT | Methylmalonic Aciduria, Mut Type |
| MVK | Mevalonic Aciduria |
| NAGLU | Sanfillipo B Disease |
| | (Mucopolysaccharidosis Type IIIB) |
| NAGS | Hyperammonemia |
| NDUFA1 | Mitochondrial Complex I |
| NDUFA2 | Mitochondrial Complex I Def (Leigh Syn) |
| NDUFA9 | Mitochondrial Complex I Def (Leigh Syn) |
| NDUFA10 | Leigh Syndrome |
| NDUFA11 | Mitochondrial Complex I |
| NDUFA12 | Mitochondrial Complex I Def (Leigh Syn) |
| NDUFAF1 | Mitochondrial Complex I Def |
| NDUFAF2 | Mitochondrial Complex I Def (Leigh Syn) |
| NDUFAF3 | Mitochondrial Complex I Def |
| NDUFAF4 | Mitochondrial Complex I Def |
| NDUFAF6 | Mitochondrial Complex I Def (Leigh Syn) |
| NDUFB3 | Mitochondrial Complex I Def |
| NDUFB9 | Mitochondrial Complex I Def |
| NDUFS1 | Mitochondrial Complex I Def |
| NDUFS2 | Mitochondrial Complex I Def |
| NDUFS3 | Mitochondrial Complex I Def (Leigh Syn) |
| NDUFS4 | Leigh Syndrome |
| NDUFS6 | Mitochondrial Complex I Def |
| NDUFS7 | Leigh Syndrome |
| NDUFS8 | Mitochondrial Complex I Def (Leigh Syn) |
| NDUFV1 | Mitochondrial Complex I Def |
| NDUFV2 | Mitochondrial Complex I Def |
| NEU1 | Sialidosis (Mucolipidosis 1) |
| NF1 | Neurofibromatosis Type 1 |
| NF2 | Neurofibromatosis Type 2 |
| NFKB2 | Immunodeificiency 10 |
| NFU1 | Multiple Mitochondrial Dysfunction Syn 1 |
| NKX2-1 | Congenital Hypothyroidism (Goiterous) |
| NKX2-5 | Congenital Hypothyroidism (Non-Goiterous 5) |
| NNT | Glucocorticoid Deficiency 4 |
| NP | Cell Mediated Immunodeficiency |
| NPC1 | Niemann-Pick Type C1 |
| NPC2 | Niemann-Pick Type C2 |
| NROB1 | Congenital Adrenal Hyperplasia |
| | (Addison Disease) |
| NSD1 | Beckwith-Wiedemann Syndrome |
| NT5C3 | Hemolytic Anemia |
| NUBPL | Mitochondrial Complex I Deficiency |
| OAT | Hyperornithinemia |
| | (Gyrate Atrophy of Choroid & Retina) |
| OGDH | α-Ketoglutaric Aciduria |
| OPA3 | 3-Methylglutaconic Aciduria Type 3 |
| | (Costeff Optic Atrophy Syndrome) |
| OPLAH | 5-Oxoprolinuria (Pyroglutamic Aciduria) |
| OTC | Hyperammonemia |
| OXCT1 | Ketoacidosis |
| PAH | Phenylketonuria |
| PAX8 | Congenital Hypothyroidism |
| | (Thyroid Dysgenesis) |
| PC | Lactic Acidemia |
| PCBD1 | Hyperphenylalaninemia |
| | (Biopterin Cofactor Defect D) |
| | (Primopterinuria) |
| PCCA | Propionic Aciduria Type 1 |
| PCCB | Propionic Aciduria Type 2 |
| PCK1 | Phosphoenolpyruvate Carboxykinase 1 |
| PCK2 | Mitochondrial PEPCK Deficiency |
| PCSK1 | Susceptibility to Obesity |
| PDHA1 | Lactic Acidemia |
| PDHB | Lactic Acidemia |
| PDHX | Lactic Acidemia |
| PDP1 | Lactic Acidemia |
| PDX1 | MODY Type 4 (Lactic Acidemia) |
| PEPD | Imidodipeptiduria |
| PET100 | Mitochondrial Complex IV Deficiency |
| PEX1 | Infantile Refsums Neonatal Adrenal |
| | Leukodystrophy Zellweger Syndrome 1 |
| PEX3 | Zellweger Syndrome |
| PEX5 | Neonatal Adrenal Leukodystrophy |
| | Zellweger Syndrome Infantile Refsums |
| PEX6 | Zellweger Syndrome Neonatal Adrenal Leukodystrophy |
| PEX7 | Rhizomelic Chondrodysplasia Punctata 1 |
| PEX10 | Zellweger Syndrome Neonatal Adrenal Leukodystrophy |
| PEX12 | Zellweger Syndrome Neonatal Adrenal Leukodystrophy |
| | Infantile Refsums |
| PEX13 | Zellweger Syndrome Neonatal Adrenal Leukodystrophy |
| PEX14 | Zellweger Syndrome |
| PEX16 | Zellweger Syndrome |
| PEX19 | Zellweger Syndrome |
| PEX26 | Zellweger Syndrome |
| PFKM | Tauri Disease (Glycogen Storage Disease VII) |
| PGM1 | Glycogen Storage Disease 14 |
| PGM2 | Glycogen Storage Disease 10 |
| PHEX | Hypophosphatemia Vit-D Resistant Rickets - Type 1 |
| PHKA1 | Glycogen Storage Disease 9D |
| PHKA2 | Glycogen Storage Disease 9A |
| PHKB | Glycogen Storage Disease 9B (8B) |
| PHKG2 | Glycogen Storage Disease 9C |
| PHYH | Refsum Disease; Phytanic Aciduria |
| PKLR | Hemolytic Anemia |
| PLOD1 | Ehlers-Danlos Type VI |
| PMM2 | Congenital Disorder of Glycosylation 1A |
| PNPO | Neonatal Epileptic Encephalopathy |
| POLG | Mitochondrial DNA Depletion Syn 4A, 4B |
| POMC | Early Onset of Obesity |
| POU1F1 | Congenital Hypothyroidism |
| | (Combined Pituitary Hormone Deficiency) |
| PPOX | Porphyria Varigata |
| PPT1 | Neuronal Ceroid Lipofuscinosis 1 |
| | (Santavuori-Haltia Disease) |
| PRKAG2 | Glycogen Storage Disease (Heart) |
| PRODH | Hyperprolinemia Type 1 (Benign) |
| PROP1 | Congenital Hypothyroidism |
| | (Combined Pituitary Hormone Deficiency) |
| PRPSAP1 | Phosphoribosyl Pyrophosphate Synthetase 1 |
| PRPSAP2 | Phosphoribosyl Pyrophosphate Synthetase 2 |
| PRPS1 | Arts Syndrome; Charcot-Marie-Tooth 5 |
| PRPS2 | Phosphoribosyl Pyrophosphate Synthetase 2 |
| PSAP | Metachromatic Leukodystrophy |
| PTPN11 | Leopard Syndrome 1 (Noonan Syndrome 1) |
| PTS | Hyperphenylalaninemia (Biopterin Cofactor Defect A) |
| PXMP3 | Infantile Refsums Zellweger Syndrome 3 |
| PYGL | Hers Disease (Glycogen Storage Disease Type 6) |
| PYGM | McArdle Syndrome (Glycogen Storage Disease Type 5) |
| QDPR | Hyperphenylalaninemia (Biopterin Cofactor Defect C) |
| RAF1 | Leopard Syndrome 2 (Noonan Syndrome 5) |
| RRM2B | Mitochondrial DNA Depletion Syn 8A, 8B |
| SARDH | Sarcosinemia (Benign) |
| SCN4B | Long QT Syndrome 10 |
| SCN5A | Long QT Syndrome 3 (Brugada Syndrome) |
| SDHA | Leigh Syndrome |
| SERAC1 | 3-Methyl-Glutaconic Aciduria |
| SERPINA1 | Emphysema/Infantile Cirrhosis |
| SGSH | Sanfillipo Syndrome A |
| | (Mucopolysaccharidosis Type IIIA) |
| SH2D1A | Lymphoproliferative Syndrome |
| (SAP) | (Duncan's Syndrome) |
| SLC2A1 | Dystonia 8, 18 |
| SLC2A2 | Fanconi-Bickel Syndrome |
| SLC2A7 | Glycogen Storage Disease 1D |
| SLC3A1 | Cystinuria-Lysinuria Type 1 |
| SLC5A5 | Congenital Hypothyroidism (Dysmorphogenesis 1) |
| SLC6A19 | Hartnup Disorder |
| SLC7A7 | Lysinuric Protein Intolerance |
| SLC7A9 | Cystinuria-Lysinuria Type 3 |
| SLC16A1 | Hyperinsulinism 7 |
| SLC17A3 | Glycogen Storage Disease Type 1C |
| SLC19A2 | Thiamine-Responsive Megaloblastic Anemia |
| SLC22A5 | Primary Systemic Carnitine Deficiency |
| SLC25A4 | Mitochondrial DNA Depletion Syn 12 |
| SLC25A13 | Citrullinemia Type 2 |
| SLC25A15 | HHH Syndrome |
| SLC25A20 | Non-Ketotic Hypoglycemia |
| SLC37A4 | Glycogen Storage Disease Type 1B |
| SLC52A1 | Riboflavin Deficiency |
| SMN1 | Spinal Muscular Atrophy |
| SMPD1 | Niemann-Pick Disease A/B |
| SNTA1 | Long QT Syndrome 12 |
| SOX3 | Panhypopituitarism |
| SPR | Hyperphenylalaninemia (Biopterin Cofactor Defect) |
| ST3GAL5 | Infantile Epilepsy Syndrome |
| STAR | Lipoid Adrenal Hyperplasia |
| SUCLA2 | Mitochondrial DNA Depletion Syn 5 |
| SUCLG1 | Mitochondrial DNA Depletion Syn 9 |
| | (Methylmalonic Aciduria) |
| SUMF1 | Multiple Sulfatase Deficiency |
| SUOX | Sulfocystinuria |
| SURF1 | Leigh Syndrome |
| TACO1 | Leigh Syndrome |
| TAT | Oculocutaneous Tyrosinemia Type 2 |
| TAZ | 3-Methylglutaconic Aciduria Type 2 (Barth Syndrome) |
| TBX19 | Adrenocorticotropic Hormone Deficiency |
| TCN2 | Methylmalonic Aciduria/Homocystinuria |
| | (Megaloblastic Anemia) |
| TG | Congenital Hypothyroidism |
| | (Dyshormonogenesis 3) |
| TH | Neurotransmitter Defect, Segawa |
| | (DOPA Responsive Dystonia) |
| THRB | Thyroid Hormone Resistance (Refetoff Syndrome) |
| TK2 | Mitochondrial DNA Depletion Syndrome 2 |
| TPO | Congenital Hypothyroidism (Dyshormonogenesis 2A) |
| TPP1 | Neuronal Ceroid Lipofuscinosis 2 |
| | (Jansky-Bielschowsky Disease) |
| TRHR | Congenital Hypothyroidism |
| TSHB | Congenital Hypothyroidism (Non-Goiterous 4) |
| TSHR | Congenital Hypothyroidism (Non-Goiterous) |
| TYMP | Mitochondrial DNA Depletion Syndrome 1 |
| | (MNGIE Syndrome) |
| TYR | Oculocutaneous Albinism |
| UGT1A1 | Unconjugated Hyperbilirubinemia |
| | Crigler-Najjar Syndrome Type 1, 2 Gilbert Syndrome |
| UMPS | Orotic Aciduria |
| UPB1 | N-Carbamyl-β-Aminoaciduria |
| UQCRB | Mitochondrial Complex III Deficiency 3 |
| UQCRC2 | Mitochondrial Complex III Deficiency 5 |
| UROD | Hepatoerythropoietic Porphyria |
| | (Porphyria Cutanea Tarda 1) |
| UROS | Congenital Erythropoietic Porphyria |
| WASP | Wiskott-Aldrich Syndrome (Immunodeficiency 2) |
| WNK4 | Pseudohypoaldosteronism 2B |
| XDH | Xanthinuria Type 1 |

TABLE 16
Molecular Genetic Autopsy Gene Panel (Cardiac Abnormalities)

| Gene | Disorder |
|---|---|
| ABCC6 | Arterial Calcification of Infacy 2 |
| ABCC9 | Cardiomyopathy, Dilated 1O (Atrial Fib 12) |
| ACE | Lt Ventricular Hypertropic Cardiomyopathy |
| ACTA2 | Aortic Aneurism 6 (Moyamoya 5) |
| ACTC1 | Cardiomyopathy, Dilated 1R (Hyper 11) |
| ACTN2 | Cardiomyopathy, Dilated 1AA |
| ADRB1 | β-1-Adrenoreceptor Deficiency |
| ADRB2 | β-2-Adrenoreceptor Deficiency |
| ADRB3 | β-3-Adrenoreceptor Deficiency |
| AKAP9 | Long QT Syndrome 11 |
| AKAP10 | Cardiac Conduction Defect |
| ANK2 | Long QT Syndrome 4 |
| ANKRD1 | Cardiac Ankrin Repeat Protein |
| APOE | Hypolipoproteinemia 3 |
| ARFGEF2 | Periventricular Hetertopia |
| BAG3 | Cardiomyopathy, Dilated 1HH |
| BMPR2 | Pulmonary Hypertension 1 |
| BRCC3 | Moyamoya Angiopathy |
| CACNA1B | Calcium Channel α-1B |
| CACNA1C | Long QT Syndrome 8 (TS1) (BS 3) |
| CACNA1D | Sinoatrial Node Dysfunction |
| CACNA2D1 | Brugada Syndrome 9 |
| CACNB2 | Brugada Syndrome 4 |
| CALM1 | Ventricular Tachycardia 4 |
| CALR3 | Cardiomyopathy, Hypertrophic 19 |
| CAMK2D | Cardiomyopathy, Dilated |
| CASQ2 | Ventricular Tachycardia 2 |
| CAV3 | Cardiomyopathy, Hypertrophic (LQT 9) |
| CFC1 | Double Outlet Right Ventricle |
| CITED2 | Ventral Septal Defect 2 (Atrial Septal 8) |
| COL4A1 | Angiopathy with Aneurisms |
| CRELD1 | Atrial Ventral Septal Defect 2 |
| CRYAB | Cardiomyopathy, Dilated 1II |
| CSRP3 | Cardiomyopathy, Dilated 1M (Hyper 12) |
| CTF1 | Cardiomyopathy, Hypertrophic |
| CTNNA3 | Right Ventricular Dysplasia 13 |
| DES | Cardiomyopathy, Dilated 1F (1I) |
| DPP6 | Ventricular Fibrillation 2 |
| DSC2 | Right Ventricular Dysplasia 11 |
| DSG2 | Cardiomyopathy, Dilated 1BB |
| DSP | Cardiomyopathy, Dilated (Epidermolysis Bullosa) |
| DTNA | Left Ventricular Noncompaction 1 |
| ELN | Supravalvular Aortic Stenosis |
| ENPP1 | Arterial Calcification of Infacy |
| EYA4 | Cardiomyopathy, Dilated 1J |
| FADD | Cardiovascular Malformation |
| FHL2 | Cardiomyopathy, Dilated 1H |
| FKTN | Cardiomyopathy, Dilated 1X |
| FOXF1 | Alveolar Capillary Dysplasia |
| GATA4 | Ventral Septal Defect 1 (Atrial Septal Def 2) |
| GATA6 | Atrial Septal Defect 5 |
| GATAD1 | Cardiomyopathy, Dilated 2B |
| GDF1 | Teratology of Fallot (Right Atrial Isomerism) |
| GJA1 | Atrterioventricular Septal Defect 3 |
| GJA5 | Atrial Fibrillation 11 |
| GNAI2 | Ventricular Tachycardia |
| GPD1L | Brugada Syndrome 2 |
| HCN1 | Voltage Gated K Channel 1 |
| HCN4 | Sinusel Brachycardia (Brugada |
| | Syndrome) |
| HEXIM1 | Cardiac Lineage Protein 1 |
| HOXD13 | VATER Association |
| ILK | Cardiac Hypertrophy |
| JPH2 | Cardiomyopathy, Hypertrophic 17 |
| JUP | Arrhythmogenic Rt Ventricular Dysplasia 11 |
| KCNA4 | Potassium Channel Cardiac Defect |
| KCNA5 | Atrial Fibrillation 7 |
| KCND3 | Brugada Syndrome 10 |
| KCNE1 | Long QT Syndrome 5 (JLN Syndrome 2) |
| KCNE1L | Voltage Gated K Channel ISV-Related 1 |
| KCNE2 | Long QT Syndrome 6 (Atrial Fibrillation 5) |
| KCNE3 | Long QT Sybdrome 10 (Brugada Syndrome 6) |
| KCNE4 | Voltage Gated K Channel ISV-Related 4 |
| KCNH2 | Long QT Syndrome 2 (SQT Syn1)(Brugada 8) |
| KCNJ2 | Long QT Syndrome 7 (ATS1)(CPVT3)(SQT3) |
| JCNJ3 | K Channel Inwardly Rectifying 3 |
| KCNJ5 | Long QT Syndrome 13 |
| KCNJ8 | Brugada Syndrome 8 |
| KCNJ11 | K Channel Inwardly Rectifying 11 |
| KCNJ12 | Cardiodysrhythmic Periodic? |
| KCNQ1 | Long QT Syndrome 1 (SQTS 2)(JNLS 1) |
| LAMA4 | Cardiomyopathy, Dilated 1JJ |
| LDB3 | Cardiomyopathy, Dilated 1C |
| LDLR | Hypercholesterolemia |
| LMNA | Cardiomyopathy, Dilated 1A (Emry-Dryfus 2, 3) |
| LRP6 | Coronary Artery Disease 2 |
| MEF2A | Coronary Artery Disease 1 |
| MIB1 | Left Ventricular Noncompaction 7 |
| MOG1 | Brugada Syndrome 11 |
| MOV10L1 | Cardiac Helicase |
| MYBPC3 | Cardiomyopathy, Dilated 1MM (Hyper CM 4) |
| MYH6 | Cardiomyopathy, Dilated 1EE |
| | (Hyper CM 6) |
| MYH7 | Cardiomyopathy, Dilated 1S (Hyper CM1) |
| MYH7B | Myosin Heavy Chain 14 |
| MYH11 | Thoracic Aortic Aneurism 4 |
| MYL2 | Cardiomyopathy, Hypertrophic 10 |
| MYL3 | Cardiomyopathy, Hypertrophic 8 |
| MYLK | Aortic Aneurism 7 |
| MYLK2 | Cardiomyopathy Hypertrophic Midventricular |
| MYLK3 | Myosin Light Chain Kinase Deficiency |
| MYOM1 | Cardiomyopathy, Hypertrophic 14 |
| MYOZ2 | Cardiomyopathy, Hypertrophic 16 |
| MYPN | Cardiomyopathy, Dilated 1KK (Hyper CM 22) |
| NEBL | Cardiomyopathy |
| NEXN | Cardiomyopathy, Dilated 1CC (Hyper CM 20) |
| NKX2-5 | Atrial Septal Defect 7 (Ventral Septal Def 3) |
| NKX2-6 | Persistent Truncus Arteriosus |
| NOTCH1 | Aortic Valva Disease 1 |
| NPPA | Atrial Fibrillation 6 |
| NUP155 | Atrial Fibrillation 10, 15 |
| PCSK9 | Hypercholesterolemia A3 |
| PDLIM3 | Dilated Cardiomyopathy |
| PKP2 | Arrhthymogenic Right Ventricular Dysplasia 9 |
| PLN | Cardiomyopathy, Dilated 1P (Hyper 18) |
| PRDM16 | Cardiomyopathy, Dilated 1LL |
| PRKAG2 | Cardiomyopathy, Hypertrophic 6 (Gyl Stor Dis) |
| PSEN1 | Cardiomyopathy, Dilated 1U |
| PSEN2 | Cardiomyopathy, Dilated 1V |
| RBM20 | Cardiomyopathy, Dilated 1DD |
| RYR2 | Arrhythmogenic Right Ventricular Dysplasia 2 |
| SCN1B | Brugada Syndrome 5 |
| SCN2B | Atrial Fibrillation 14 |
| SCN3B | Brugada Syndrome 7 (Atrial Fibrillation 12) |
| SCN4B | Long QT Syndrome 10 |
| SCN5A | Cardiomyopathy, Dilated 1E (BS 1)(LQTS3) |
| SDHA | Cardiomyopathy, Dilated 1GG |
| SGCD | Cardiomyopathy, Dilated 1L (LGMD 2F) |
| SLC2A10 | Arterial Tortuosity Syndrome |
| SMAD6 | Aortic Valve Disease 2 |
| SNTA1 | Long QT Syndrome 12 |
| TAB2 | Congenital Heart Defects 2 |
| TBX20 | Atrial Septal Defect 4 |
| TCAP | Cardiomyopathy, Dilated 1N (LGMD 2F) |
| TGFB3 | Arrhythmogenic Right Ventricular Dysplasia 1 |
| TIN | Cardiomyopathy, Dilated 1G |
| TMPO | Cardiomyopathy, Dilated 1T |
| TNNC1 | Cardiomyopathy, Dilated 1Z (Hyper CM 13) |
| TNNI3 | Cardiomyopathy, Dilated 1FF (2A) (Hyper CM 7) |
| TNNT2 | Cardiomyopathy, Dilated 1D (Hyper CM 2) |
| TPM1 | Cardiomyopathy, Dilated 1Y (Hyper CM 3) |
| TRDN | Ventricular Tachycardia 5 |
| TRIM63 | Cardiomyopathy, Heterotrophic 15 |
| TRPM4 | Progressive Heart Block 1B |
| TTN | Cardiomyopathy, Hypertrophic 9 (Dilated CM 1G) |
| VCL | Cardiomyopathy, Dilated 1W (Hyper CM 15) |
| ZFPM2 | Teratology of Fallot |
| ZIC3 | Congenital Heart Defects 1 (Visceral Heterotaxy) |

TABLE 17
Molecular Genetic Autopsy Gene Panel
(Inborn Errors with Cardiac Symptoms)

| Gene | Disorder |
|---|---|
| AARS2 | Combined Oxidative Phosphorylation Defic 8 |
| ABCA1 | High Density Lipoprotein Deficiency |
| ADK | Adenosine Kinase Deficiency |
| ACAD8 | Isobutyryl-CoA Dehydrogenase Deficiency 8 |
| ACAD9 | Mitochondrial Complex I Deficiency 9 |
| ACADL | Long-Chain Acyl-CoA Dehydrogenase Def |
| ACADVL | Very Long Chain Acyl-CoA Dehydrogenase Def |
| AGK | Mitochondrial DNA Depletion Syn 10 |
| AGL | Glycogen Storage Disease 3A, 3B, 3C |
| AGXT | Hyperoxaluria 1 (Glycolic Aciduria) |
| ALG12 | Congenital Disorder of Glycosylation 1G |
| APOA1 | Amyloidosis 3 |
| APOA2 | Systemic Amyloidosis |
| APOB | Hypocholesterolemia |
| ATP5E | Mitochondrial Complex V Deficiency 3 |
| ATP7B | Wilson's Disease |
| ATPAF2 | Mitochondrial Complex V Deficiency 1 |
| BOLA3 | Multiple Mitochondrial Dysfunction Syn 2 |
| C10orf2 | Mitochondrial DNA Depletion Syndrome 7 |
| CBS | Homocystinuria (Cystathioninuria β Synthase) |
| COA5 | Mitochondrial Complex IV Deficiency |
| COG7 | Congenital Disorder of Glycosylation 2E |
| COQ9 | Coenzyme Q10 Deficiency 5 |
| COX10 | Mitochondrial Complex IV Deficiency |
| COX15 | Cytochrome C Oxidase Deficiency 2 |
| CPT1A | Carnitine Palmotyltransferase 1 Deficiency |
| CPT2 | Carnitine Palmotyltransferase 2 Deficiency |
| CTSA | β-Galactosidase Deficiency |
| D2HGDH | D-2-Hydroxy-Glutaric Aciduria |
| DOLK | Congenital Disorder of Glycosylation 1M |
| DNAJC19 | 3-Methyl-Glutaconic Aciduria 5 |
| DPM3 | Congenital Disorder of Glycosylation 1O |
| DSC1 | Desmocollin Protein |
| EARS2 | Combined Oxidative Phosphorylation Defect 12 |
| ELAC2 | Combined Oxidative Phosphorylation Defect 17 |
| EPHX2 | Hypercholesterolemia |
| ETFA | Glutaric Aciduria 2A |
| ETFB | Glutaric Aciduria 2B (Early Onset) |
| ETFDH | Glutaric Aciduria 2C |
| FOXRED1 | Mitochondrial Complex I Deficiency (Infantile) |
| GAA | Glycogen Storage Disease II (Pompe) |
| GBA | Gaucher Disease 1, 2, 3 |
| GBE1 | Glycogen Storage Disease IV |
| GHR | Hypercholesterolemia |
| GLA | Fabry Disease |
| GLB1 | GM1 Gangliosidosis 1, 2, 3 (MPS 4B) |
| GLUL | Glutamate Ammonia Ligase Deficiency |
| GYG1 | Glycogen Storage Disease 15 |
| GNPTAB | Mucolipidosis 2, 3A (I Cell Disease) |
| GSBS | Hypercholesterolemia |
| GSN | Amyloidosis (Finnish) |
| GUSB | Mucopolysaccharidosis 7 (Sly Disease) |
| GYS1 | Glycogen Storage Disease 0 (Muscle) |
| HADH | 3-Hydroxy-Acyl-CoA Dehydrogenase Def |
| HADHA | 3-OH-Long-Chain Acyl-CoA Dehydrogenase |
| HADHB | Mitochondrial Trifunctional Protein Deficiency |
| HFE | Hemochromatosis |
| HFE2 | Hemochromatosis 2A (Juvenile) |
| HGD | Alkaptonuria (Homogentisic Oxidase Def |
| HMBS | Acute Intermittent Prophyria |
| HTR4 | Serotonin Receptor 4 |
| IDH2 | D-2-Hydroxy-Glutaric Aciduria 2 |
| IDUA | Mucopolysaccharidosis 1h (Hurler |
| | Syn) |
| ITIH4 | Hypercholesterolemia |
| LAMP2 | Glycogen Storage Dis 2 (Danon Disease) |
| LIAS | Lipoic Acid Synthetase Deficiency |
| LPA | Congenital Apolipoproteinemia |
| LPL | Combined Hyperlipidemia 1 |
| MGME1 | Mitochondrial DNA Depletion Syn 11 |
| MLYCD | Malonic Aciduria (Malonyl-CoA Decarboxylase) |
| MMACHC | Methylmalonic/Homocystinuria CblC |
| MRPL3 | Combined Oxidative Phosphorylation Def 9 |
| MRPL44 | Combined Oxidative Phosphorylation Def 16 |
| MRPS22 | Combined Oxidative Phosphorylation Def 5 |
| MTO1 | Combined Oxidative Phosphorylation Def 10 |
| MUT | Methylmalonic Aciduria |
| NDUFA1 | Mitochondrial Complex I Deficiency |
| NDUFA11 | Mitochondrial Complex I Deficiency |
| NDUFAF1 | Mitochondrial Complex I Deficiency |
| NDUFAF2 | Mitochondrial Complex I Deficiency 3 |
| NDUFAF3 | Mitochondrial Complex I Deficiency 6 |
| NDUFAF4 | Mitochondrial Complex I Deficiency |
| NDUFB3 | Mitochondrial Complex I Deficiency |
| NDUFB9 | Mitochondrial Complex I Deficiency |
| NDUFS1 | Mitochondrial Complex I Deficiency |
| NDUFS2 | Mitochondrial Complex I Deficiency |
| NDUFS3 | Mitochondrial Complex I Deficiency |
| NDUFS4 | Mitochondrial Complex I Deficiency 1 |
| NDUFS6 | Mitochondrial Complex I Deficiency 2 |
| NDUFV1 | Mitochondrial Complex I Deficiency |
| NDUFV2 | Mitochondrial Complex I Deficiency |
| NOS1AP | Nitric Oxide Synthase 1 |
| NUBPL | Mitochondrial Complex I Deficiency |
| PCCA | Propionic Acidemia A |
| PCCB | Propionic Acidemia B |
| PDSS1 | Coenzyme Q10 Deficiency 2 |
| PFKFB2 | 6-Posphofructo-1-kinase Deficiency |
| PNPLA2 | Neutral Lipid Storage Disease |
| PMM2 | Congenital Disorder of Glycosylation 1A |
| SCO2 | Cytochrome C Oxidase Deficiency |
| SCNN1A | Pseudohypoaldosteronism 1 |
| SCNN1B | Pseudohypoaldosteronism 1 |
| SCNN1G | Pseudohypoaldosteronism 1 |
| SDHAF1 | Mitochondrial Complex II Deficiency |
| SLC22A5 | Primary Systemic Carnitine Def (Startle Syn 1) |
| SLC25A3 | Mitochondrial Phosphate Carrier Deficiency |
| SLC25A4 | Mitochondrial DNA Depletion Syn 12 |
| SLC25A20 | Carnitine Acylcarnitine Translocase Deficiency |
| SMN1 | Spinal Muscular Atrophy |
| TAZ | Cardiomyopathy, Dilated 3A (3-Me-Glutaconic Acid) |
| TMEM70 | Mitochondrial Complex V Deficiency 2 |
| TPI1 | Triosephosphate Isomerase Deficiency 1 |
| TSFM | Combined Oxidative Phosphorylation Def 3 |
| TSPYL1 | SIDS with Dysgenesis of Testes Syndrome |
| TXNRD2 | Thioredoxin Reductase 2 |

TABLE 18
Molecular Genetic Autopsy Gene Panel
(Syndromes with Cardiac Symptoms)

| Gene | Disorder |
|---|---|
| ACTA1 | Nemaline Myopathy Disease 3 |
| ACVR2B | Visceral Heterotaxy 4 |
| ACVRL1 | Hemorrhagic Telangiectasia 2 |
| ADAMTSL2 | Geleophysic Dysplasia 1 |
| ALMS1 | Alstrom Syndrome |
| ANKS6 | Nephronophthisis 16 |
| ARHGAP31 | Adams-Oliver Syndrome 1 |
| B3GALT6 | Ehlers-Danlos Syndrome 2 |
| B3GALTL | Peters-Plus Syndrome |
| B3GAT3 | Larson-like Syndrome |
| BRAF | Noonan Syndrome 7 |
| BSCL2 | Congenital Generalized Lipodystrophy 2 |
| CACNA1S | Thyrotoxic Periodic Paralysis 1 |
| CAPN3 | Limb Girdle Muscular Dystrophy 2A |
| CAV1 | Congenital Generealized Lipodystrophty 3 |
| CBL | Noonan-Like Syndrome |
| CCBE1 | Lymphangiectasia-Lymphoma Syndrome |
| CCDC11 | Visceral Heterotoxy 6 |
| CCDC114 | Ciliary Dyskenesia 20 |
| CD96 | C Syndrome |
| CDKN1C | Beckwith-Wiedemann Syndrome |
| CHD7 | CHARGE Syndrome |
| CHKB | Congenital Muscular Dystrophy 1E |
| CHST14 | Ehlers-Danlos Syndrome 1 |
| CLIC2 | Mental Retardation Syndrome 32 |
| CNBP | Myotonic Dystrophy 2 |
| COL1A2 | Ehlers-Danlos Syndrome 6, 7A, 11 |
| COL3A1 | Ehlers-Danlos Syndrome 3, 4 |
| COL5A2 | Ehleers-Danlos Syndrome 1B, 2 |
| COL7A1 | Epidermolysis Bullosa |
| CREBBP | Rubenstein-Taybi Syndrome |
| CRTAP | Osteogenesis Imperfecta 7 |
| DHCR7 | Smith-Lemly-Opitz Syndrome |
| DMD | Duchenne/Becker Muscular Dystrophy |
| DMPK | Myotonic Dystrophy 1 |
| DNM1L | Encephalopathy (Dynamin-Lite Protein) |
| DSE | Ehlers-Danlos Syndrome 2 |
| ECE1 | Hirschsprungs with Cardiac Defects |
| EFEMP2 | Cutis Laxa 1B |
| EFTUD2 | Mandibulofacial Dysostosis |
| EMD | Emry-Dryfus Muscular Dystrophy 1 (AF 13) |
| ENG | Rendu-Osler-Weber Disease 1 |
| EOGT | Adams-Oliver Syndrome 4 |
| EPG5 | Vici Syndrome |
| ERCC8 | Cockayne Syndrome A |
| ESCO2 | Roberts Syndrome |
| EVC | Ellis van Crevald Syndrome 1 |
| EVC2 | Ellis van Crevald Syndrome 2 |
| F5 | Thrombophilia (Factor 5 Leiden) |
| FANCA | Fanconi Anemia A |
| FBLN5 | Cutis Laxa 1A, 2 |
| FBN1 | Geleophysic Dysplasia 2 (Weil-Marchesani S) |
| FBN2 | Arthrogryposis 9 |
| FGFR1 | Hypogonadotropic Hypogonadism |
| FGFR2 | Saethre-Chotzen Syndrome |
| FHL1 | Emry-Dryfus Muscular Dystrophy 6 |
| FIG4 | Charcot-Marie-Tooth Syndrome 1 |
| FKRP | Congenital Muscular Dystrophy A5, C |
| FLNA | Otopalatodigital Syndrome 1, 2 |
| FLNC | Distal Myopathy 4 |
| FOXC1 | Axenfeld-Rieger Syndrome 3 |
| FOXC2 | Lymphadema-Distichiasis Syndrome |
| FXN | Friedreich Ataxia |
| GMPPB | Muscular Dystrophy Congenital A14, B14, C14 |
| GPC3 | Simpson-Golabi-Behmel Syndrome |
| H19 | Beckwith-Wiedemann Syndrome |
| HCCS | Microphthalmia |
| HOXA1 | Bosley-Salih-Alorainy Syndrome |
| HRAS | Costello Syndrome |
| ITPKC | Kawasaki Syndrome |
| JAG1 | Alagille Syndrome |
| KCNQ2 | Epileptic Encephalopathy 7 |
| KDM6A | Kabuki Syndrome 2, 3 |
| KIAA0196 | Ritscher-Schinzel Sydrome |
| KRAS | Noonan Syndrome 3 |
| MAP2K1 | Cardio-Facio-Cutaneous Syndrome 3 |
| MAP2K2 | Cardio-Facio-Cutaneous Syndrome 4 |
| MECP2 | Rett Syndrome (MR Syn 28, 31) |
| MEGF8 | Carpenter Syndrome 2 |
| MID1 | Opitz-GBBB Syndrome 1 |
| MKKS | McKusick-Kaufman Syn (Bardet-Biedl Syn 6) |
| MYCN | Feingold Syndrome 1 |
| MYO61 | Deafness with Hypertrophic Cardiomyopathy |
| MYOT | Limb-Girdle Muscular Dystrophy 1A |
| NIPBL | Cornelia de Lange 1 |
| NODAL | Visceral Heterotaxy 5 |
| NOTCH2 | Hajdu-Cheny Syndrone |
| NPHP3 | Meckel Syndrome 1 |
| NRAS | Noonan Syndrome 6 |
| NSD1 | Beckwith-Wiedemann Syndrome (Sotos Syn 1) |
| NSDHL | CHILD Syndrome (CK Syndrome) |
| OFD1 | Oral-Facial-Digital Syndrome 1 (Jobert Syn 10) |
| PHYH | Refsun Disease |
| PIGA | Multiple Congenital Anomalies 2 |
| PIGL | CHIME Syndrome |
| PIGN | Multiple Congenital Anomalies 1 |
| PLEC | Limb Girdle Muscular Dystrophy 2Q |
| POLG | Progressive Ophthalmoplegia 1 |
| POLG2 | Progressive Ophthalmoplegia 4 |
| POMT1 | Limb Girdle Muscular Dystrophy 2K |
| POMT2 | Limb Girdle Muscular Dystrophy 2N |
| PRKAR1A | Intracardiac Myxoma (Carney Complex 1) |
| PROC | Thrombophilia (Protein C Deficiency) |
| PRRX1 | Agnathia-Otocephaly Complex |
| PTEN | Cowden Syndrome |
| PTPN11 | Noonan Syndrome 1 (Leopard Syndrome) |
| PTRF | Congenital Lipodystrophy 4 (LQTS) |
| PUF60 | Verheij Syndrome |
| RAB23 | Carpenter Syndrome (Acrocephalosyndactyly 2) |
| RAF1 | Noonan Syndrome 5 (Leopard Syndrome 2) |
| RAI1 | Smith-Magenis Syndrome |
| RARB | Microphthalmia Syndrome 12 |
| RBM8A | Thrombocytopenia |
| RBM10 | TARP Syndrome |
| RIT1 | Noonan Syndrome 8 |
| ROR2 | Robinow Syndrome (Brachydactyly B1) |
| RPL5 | Blackfan-Diamond Syndrome 6 |
| RPL11 | Blackfan-Diamond Syndrome 7 |
| RPL15 | Blackfan-Diamond Syndrome 12 |
| RPL26 | Blackfan-Diamond Syndrome 11 |
| RPL35A | Blackfan-Diamond Syndrome 5 |
| RPS7 | Blackfan-Diamond Syndrome 8 |
| RPS10 | Blackfan-Diamond Syndrome 9 |
| RPS17 | Blackfan-Diamond Syndrome 4 |
| RPS19 | Blackfan-Diamond Syndrome 1 |
| RPS24 | Blackfan-Diamond Syndrome 3 |
| RPS26 | Blackfan-Diamond Syndrome 10 |
| RPSA | Congenital Aplasia |
| RYR1 | Mlignant Hyperthermia 1 |
| SALL1 | Townes-Brocks Syndrome |
| SLMAP | Sarcolema Associated Protein |
| SEMA3E | CHARGE Syndrome 2 |
| SEPN1 | Rigid Spine Muscular Dystrophy 1 |
| SETBP1 | Schinzel-Giedion Syndrome |
| SGCA | Limb Girdle Muscular Dystrophy 2D |
| SGCB | Limb Girdle Muscular Dystrophy 2E |
| SGCG | Limb Girdle Muscular Dystrophy 2C |
| SH3PXD2B | Frank-Ter Haar Syndrome |
| SHOC2 | Noonan-Like Syndrome |
| SKI | Shprintzen-Goldberg Syndrome |
| SKIV2L | Trichohepatoenteric Syndrome 2 |
| SLC19A2 | Thiamine Responsive Megaloblastic |
| | Anemia |
| SMAD3 | Loeys-Dietz Syndrome 3 |
| SMAD4 | Myhre Syndrome |
| SOS1 | Noonan Syndrome 4 |
| SOX2 | Microphthalmia Syndrome 3 |
| SPRED1 | Legires Syndrome |
| STAMBP | Microcephaly-Capillary Malformation Syn |
| STK4 | T Cell Immunodeficiency Cardiac Manifestations |
| STRA6 | Microphthalmia Syndrome 8, 9 |
| SYNE1 | Emry-Dryfus Muscular Dystrophy 4 |
| SYNE2 | Emry-Dryfus Muscular Dystrophy 5 |
| TBC1D24 | Early Infantile Epileptic Encephalopathy 16 |
| TBX1 | DiGeorge Syndrome |
| TBX3 | Ulnar-Mammary Syndrome |
| TBX5 | Holt-Oram Syndrome 1 |
| TERT | Dyskeratosis Congenita 2, 3, 4 |
| TGFBR1 | Loeys-Dietz Syndrome 1A |
| TGFBR2 | Loeys-Dietz Syndrome 1B, 2B |
| TGFBR3 | Loeys-Dietz Syndrome 3 |
| TMEM43 | Emry-Dryfus Muscular Dystrophy 7 |
| TPM3 | Congenital Nemaline Myopathy 1 |
| TSC1 | Tuberous Sclerosis 1 |
| TTC37 | Trichohepatoenteric Syndrome 1 |
| TTR | Amyloidosis 7 |
| TWIST1 | Saethre-Chotzen Syndrome |
| UBR1 | Johanson-Blizzard Syndrome |
| WT1 | Meacham Syndrome, Frasier Syndrome |
| XK | McLeod Syndrome/Granulomatous Disease |
| YARS2 | Myopathic Sideroblastic Anemia |
| ZEB2 | Mowat-Wilson Syndrome |
| ZNF469 | Brittle Cornea Syndrome |

TABLE 19
Molecular Genetic Autopsy Gene Panel (Mitochondrial
Genes with Cardiac Symptoms)

| Gene | Disorder |
|---|---|
| MT-ATP6 | Cardiomyopathy, Hypertrophic 10 |
| MT-ATP8 | Cardiomyopathy, Hypertrophic 8 |
| MT-ND1 | Cardiomyopathy, Hypertrophic 5 (MELAS) |
| MT-ND4 | MELAS |
| MT-ND5 | MELAS; MERRF; Leigh's Syndrome 2 |
| MT-ND6 | Leber Optic Neuropathy; Leigh's Syndrome |
| MT-TD | Mitochondrial tRNA - ASP |
| MT-TG | Mitochondrial tRNA - GLY |
| MT-TH | Mitochondrial tRNA - HIS (MERFF) |
| MT-TI | Mitochondrial tRNA - ILE (MELAS) |
| MT-TK | Mitochondrial tRNA - LYS (MERFF) |
| MT-TL1 | Mitochondrial tRNA - LEU1 (MERFF; MELAS) |
| MT-TL2 | Mitochondrial tRNA - LEU2 (Dilated CM) |
| MT-TM | Mitochondrial tRNA - MET |
| MT-TQ | Mitochondrial tRNA - GLU |
| MT-TS1 | Mitochondrial tRNA - SER1 (MERFF; MELAS) |
| MT-TS2 | Mitochondrial tRNA - SER2 (MERFF) |

Some exemplary disorders and genes can be used for a low-cost primary newborn screen. ALDOB—Hereditary Fructose Intolerance (frequency 1/20,000). Fructose intolerance can become apparent in infancy at the time of weaning, when fructose or sucrose is added to the diet. Clinical features can include recurrent vomiting, abdominal pain, and hypoglycemia that may be fatal. Long-term exposure to fructose can result in liver failure, renal tubulopathy, and growth retardation. Treatment can involve the restriction of fructose in the patient's diet. ATP7A—Menke Disease (frequency 1/40,000). Menke disease is an X-linked recessive disorder characterized by generalized copper deficiency. The clinical features can result from the dysfunction of several copper-dependent enzymes. Treated from early infancy with parenteral copper-histidine can result in normal or near-normal intellectual development. ATP7B—Wilson Disease frequency 1/33,000). Wilson disease is an autosomal recessive disorder characterized by dramatic build-up of intracellular hepatic copper with subsequent hepatic and neurologic abnormalities. Treatment can be with a chelating agent such as penicillamine or triethylene tetramine. Orthotropic liver transplantation can also been used. CTNS—Cystinosis frequency 1/100,000-1,200,000). Cystinosis can been classified as a lysosomal storage disorder on the basis of cytology and other evidence pointing to the intralysosomal localization of stored cystine. Cystinosis can differ from the other lysosomal diseases inasmuch as acid hydrolysis, the principal enzyme function of lysosomes, is not known to play a role in the metabolic disposition of cystine. Children with cystinosis treated early and adequately with cysteamine can have renal function that increases during the first 5 years of life and then declines at a normal rate. Patients with poorer compliance and those who are treated at an older age can do less well. DHCR7—Smith Lemli Opitz Syndrome (frequency 1/20,000-1/30,000). Smith-Lemli-Opitz syndrome is an autosomal recessive multiple congenital malformation and mental retardation syndrome due to a deficiency of 7-dehydrocholesterol reductase. Treatment with dietary cholesterol can supply cholesterol to the tissues and also reduce the toxic levels of 7-dehydrocholesterol. The impact on the families of some SLOS children and adults can be profound when their cholesterol deficiency syndrome was treated. In some cases, growth improves, older children learn to walk, and adults speak for the first time in years. How much better the children feel can be important. NDN and SNRPN—Prader Willi Syndrome (frequency is 1/25,000) Prader-Willi syndrome can be characterized by diminished fetal activity, obesity, muscular hypotonia, mental retardation, short stature, hypogonadotropic hypogonadism, and small hands and feet. It can be considered to be an autosomal dominant disorder and can be caused by a micro deletion or disruption of a gene or several genes on the proximal long arm of the paternal chromosome 15 or maternal uniparental disomy 15, because the gene(s) on the maternal chromosome(s) 15 can be inactive through imprinting. Growth hormone treatment can accelerate growth, decrease percent body fat, and/or increase fat oxidation, but does not significantly increase resting energy expenditure. Improvements in respiratory muscle strength, physical strength, and agility have also been observed, suggesting that growth hormone treatment may have value in reducing disability in children with PWS. SERPINA 1—Alpha-1 Anti-Trypsin Deficiency (frequency in European populations 1/2,500). Alpha-1-antitrypsin deficiency is an autosomal recessive disorder. The most common manifestation is emphysema, which becomes evident by the third to fourth decade. A less common manifestation of the deficiency is liver disease, which occurs in children and adults, and may result in cirrhosis and liver failure. The autophagy-enhancing drug carbamazepine can decrease the hepatic load of mutant alpha-1-antitrypsin Z protein. A combination of zinc finger nucleases and piggyBac technology in human induced pluripotent stem cells can achieve biallelic correction of a point mutation (glu342 to lys) in the alpha-1-antitrypsin gene. GAA—Glycogen Storage Disease II (Pompe Disease, frequency is 1/40,000). Glycogen storage disease an autosomal recessive disorder, is the prototypic lysosomal storage disease. In the classic infantile form (Pompe disease), cardiomyopathy and muscular hypotonia can be the cardinal features. It can be due to a deficiency of alpha-1,4-glucosidase, a lysosomal enzyme involved in the degradation of glycogen within cellular vacuoles. Enzyme replacement therapy with alglucosidase-alfa can be effective, particularly in infants. GALAC—Krabbe Disease (Globoid Cell Leukodystrophy, frequency is 1/100,000). Krabbe disease, due to galactosylceramidase deficiency, is an autosomal recessive lysosomal disorder affecting the white matter of the central and peripheral nervous systems. Patients can present within the first 6 months of life with extreme irritability, spasticity, and developmental delay. Treatment can involve allogeneic hematopoietic stem cell transplantation. GBA—Gaucher Disease. Gaucher Disease is an autosomal recessive lysosomal storage disorder due to a deficiency of acid beta-glucocerebrosidase, also known as beta-glucosidase, a lysosomal enzyme that catalyzes the breakdown of the glycolipid glucosylceramide to ceramide and glucose. There can be intracellular accumulation of glucosylceramide within cells of mononuclear phagocyte origin. It can be categorized phenotypically into 3 main subtypes: nonneuronopathic type I, acute neuronopathic type II, and subacute neuronopathic type III. Type I is the most common form and lacks primary central nervous system involvement. Types II and III have central nervous system involvement and neurologic manifestations. All 3 forms can be caused by mutations in the GBA gene. There can be 2 additional phenotypes which may be distinguished: a perinatal lethal form, which is a severe form of type II, and type IIIC, which also can have cardiovascular calcifications. The primary form of therapy can involve enzyme replacement involving the use of modified glucocerebrosidase (Alglucerase or Ceredase). The Frequency in the Ashkenazi Jewish population is 1/2,500 and 1/300,000 on the general European population. IDS—Hunter Syndrome (Mucopolysaccharidosis II, frequency is 1/100,000 male births). Mucopolysaccharidosis II is an X-linked recessive disorder caused by deficiency of the lysosomal enzyme iduronate sulfatase, leading to progressive accumulation of glycosaminoglucans in nearly all cell types, tissues, and organs. Patients with MPS II can excrete excessive amounts of chondroitin sulfate B (dermatan sulfate) and heparitin sulfate (heparan sulfate) in the urine. Treatment with intravenous enzyme replacement therapy may halt or possibly improve brain MRI abnormalities in patients with MPS. IDUA—Hurler/Schie Syndrome (Mucopolysaccharidosis I, frequency is 1/100,000 newborns). Deficiency of alpha-L-iduronidase can result in a wide range of phenotypic involvement with 3 major recognized clinical entities: Hurler (MPS IH), Scheie (MPS IS), and Hurler-Scheie (MPS IH/S) syndromes. Hurler and Scheie syndromes represent phenotypes at the severe and mild ends of the MPS I clinical spectrum, respectively, and the Hurler-Scheie syndrome is intermediate in phenotypic expression. Treatment can involve bone marrow transplantation and enzyme replacement therapy. SLC7A7—Lysinuric Protein Intolerance (frequency is 1/60,000). Lysinuric protein intolerance can be caused by defective cationic amino acid (CAA) transport at the basolateral membrane of epithelial cells in kidney and intestine. Metabolic derangement can be characterized by increased renal excretion of CAA, reduced CAA absorption from intestine, and orotic aciduria. Treatment can include protein-restricted diet and supplementation with oral citrulline therapy which results in a substantial increase in protein tolerance, striking acceleration of linear growth, as well as increase in bone mass.

It should be understood from the foregoing that, while particular implementations have been illustrated and described, various modifications can be made thereto and are contemplated herein. An embodiment of one aspect of the disclosure can be combined with or modified by an embodiment of another aspect of the disclosure. It is not intended that the invention(s) be limited by the specific examples provided within the specification. While the invention(s) has (or have) been described with reference to the aforementioned specification, the descriptions and illustrations of embodiments of the invention(s) herein are not meant to be construed in a limiting sense. Furthermore, it shall be understood that all aspects of the invention(s) are not limited to the specific depictions, configurations or relative proportions set forth herein which depend upon a variety of conditions and variables. Various modifications in form and detail of the embodiments of the invention(s) will be apparent to a person skilled in the art. It is therefore contemplated that the invention(s) shall also cover any such modifications, variations and equivalents.

What is claimed is:

1. A method of detecting a genetic condition in a subject, comprising:
(a) providing a sample previously obtained from the subject, wherein the sample comprises a dried blood spot (DBS) sample, a cord blood sample, single blood drop, saliva, or oral swab;
(b) sequencing the sample to generate a sequencing product, wherein the sequencing product is determined by a sequencing method selected from a group consisting of next-generation sequencing (NGS), targeted next-generation sequencing (TNGS) and whole-exome sequencing (WES); and
(c) analyzing the sequencing product to determine a presence of, absence of or predisposition to the genetic condition.

2-3. (canceled)

4. The method of claim 1, wherein the subject is a fetus, a newborn, an infant, a child, an adolescent, a teenager or an adult.

5-7. (canceled)

8. The method of claim 1, wherein the sample is a dried blood spot (DBS) sample.

9. The method of claim 1, wherein the sample contains less than 50 μL of blood.

10. The method of claim 1, wherein providing a sample comprises isolating more than 7 pg of DNA from the sample.

11. The method of claim 1, wherein providing a sample comprises isolating less than 1 μg of DNA from the sample.

12. (canceled)

13. The method of claim 11, wherein more than 80% of the isolated DNA is double stranded DNA.

14-19. (canceled)

20. The method of claim 1, wherein the genetic condition is caused by a genetic alteration and wherein the genetic alteration is selected from a group consisting of the following: nucleotide substitution, insertion, deletion, frameshift, nonframeshift, intronic, promoter, known pathogenic, likely pathogenic, splice site, gene conversion, modifier, regulatory, enhancer, silencer, synergistic, short tandem repeat, genomic copy number variation, causal variant, genetic mutation, and epigenetic mutation.

21. The method of claim 20, wherein analyzing the sequencing product comprises determining a presence, absence or predisposition of the genomic copy number variation or the genetic mutation.

22-23. (canceled)

24. The method of claim 20, further comprising verifying cis- or trans-configuration of the genetic mutation using a next-generation sequencing (NGS) or an orthogonal method, wherein the genetic mutation is a heterozygous mutation.

25-35. (canceled)

36. The method of claim 1, wherein the subject is in a neonatal intensive care unit (NICU), pediatric center, pediatric clinic, referral center or referral clinic.

37. (canceled)

38. The method of claim 1, wherein a Newborn Screening (NBS) has been performed on the subject.

39. The method of claim 1, wherein sequencing the DNA comprises sequencing at least one gene selected from any one of Tables 3, 4, 13, 14, 15, 16, 17, 18, or 19.

40-41. (canceled)

42. The method of claim 1, wherein analyzing the sequencing product further comprises comparing the sequencing product with a database of neonatal specific variant annotation.

43-45. (canceled)

46. A kit, comprising at least one capture probe targeting to at least one gene selected from any one of Tables 3, 4, 13, 14, 15, 16, 17, 18, or 19.

47. (canceled)

48. The kit of claim 46, wherein the at least one capture probe is used for solution hybridization or DNA amplification.

49. The kit of claim 46, further comprising at least one support bearing the at least one capture probe.

50. The kit of claim 49, wherein the at least one support comprises a microarray or a bead.

51. A system comprising:
a) a digital processing device comprising an operating system configured to perform executable instructions and a memory device; and
b) a computer program including instructions executable by the digital processing device to classify a sample from a subject or a relative of the subject comprising:
i) a software module configured to receive a sequencing product from the sample from the subject or a relative of the subject;
ii) a software module configured to analyze the sequencing product from the sample from the subject or a relative of the subject; and
iii) a software module configured to determine a presence, absence or predisposition of a genetic condition.

52. The system of claim 51, wherein the subject is a newborn.

53. (canceled)

54. The system of claim 51, wherein the software module is configured to automatically detect the presence, absence or predisposition of a genetic condition.

55. (canceled)


SEQUENCE LISTING (1 sequence)

SEQ ID NO: 1 - DNA, 56 bp, Artificial Sequence
  Description of Artificial Sequence Synthetic oligonucleotide
aggtcgttacgtacgctacgacctacatcagtacataggcatgcaaagctaggtgt